(19) United States
(12) Patent Application Publication
(10) Pub. No.: US 2008/0311040 A1
(43) Pub. Date: Dec. 18, 2008

(54) METHODS AND COMPOSITIONS FOR IMPROVED THERAPEUTIC EFFECTS WITH siRNA

(75) Inventors: David Arthur Berry, Brookline, MA (US); Noubar Boghes Afeyan, Lexington, MA (US); Chris Varma, Lexington, MA (US)

Correspondence Address: Noel E. Day;Miller, Canfield, Paddock & Stone, Suite 5000, 277 South Rose Street, Kalamazoo, MI, 49007, US

(73) Assignee: Flagship Ventures, Cambridge, MA (US)

(21) Appl. No.: 12/043,029

(22) Filed: Mar. 5, 2008

Related U.S. Application Data
(60) Provisional application No. 60/893,165, filed on Mar. 6, 2007.

(51) Int. Cl. A61K 49/00; A61K 31/70; C12N 5/06; A61K 38/43; G06G 7/48; A61P 43/00; A61K 38/00; A61K 39/395; C12Q 1/68

(52) U.S. Cl. 424/9.1; 514/44; 435/375; 435/6; 424/178.1; 424/94.1; 514/54; 514/2; 703/11

(57) ABSTRACT

The present invention relates to chemically modified, linked double-stranded (ds)RNA compositions comprising two or more double-stranded (ds) oligoribonucleotides linked by at least one linking moiety and methods of formulating and delivering such compositions to modulate gene expression through target-specific RNA co-interference (RNAco-i). The compositions of the invention may optionally comprise a conjugation or a complex with one or more small molecule drugs, protein therapeutics, or other dsRNA molecules. The present invention is directed at the methods of production for, methods of use of, and therapeutic utilities for RNAi co-interference therapy utilizing the compositions of the invention.

This application claims priority to U.S. Application No. 60/893,165 filed on Mar. 6, 2007, the content of which is incorporated herein in its entirety by reference.

### BRIEF DESCRIPTION OF THE INVENTION

The present invention relates to chemically modified, linked double-stranded (ds)RNA compositions comprising two or more double-stranded (ds) oligoribonucleotides linked by at least one linking moiety and methods of formulating and delivering such compositions to modulate gene expression through target-specific RNA co-interference (RNAco-i). The compositions of the invention may optionally comprise a conjugation or a complex with one or more small molecule drugs, protein therapeutics, or other dsRNA molecules. The present invention is directed at the methods of production for, methods of use of, and therapeutic utilities for RNAi co-interference therapy utilizing the compositions of the invention. The compositions and methods of the invention uniquely enable the development of novel therapies that target two hybridization sequences of a target nucleic acid or two or more nucleic acid targets through a multinodal molecule capable of RNA co-interference upon delivery to a cell or an organism in need of such novel therapy. These improvements are aimed at affording greater efficacy and, in some embodiments, synergistic treatments of a disease state and/or to reduce drug-associated toxicities. Finally, the present invention describes novel compositions, including pharmaceutical compositions, for the delivery of multiple dsRNAs simultaneously.

### BACKGROUND

The following is a discussion of relevant art pertaining to RNA-interference (RNAi). The discussion is provided only for understanding of the invention that follows. The summary is not an admission that any of the work described below is prior art to the claimed invention. RNA interference (RNAi) is premised on the introduction of particular dsRNA or small interfering RNA (siRNA) molecules into organisms such as C. elegans (Fire et al., 1998), which can lead to the silencing of specific genes that are highly homologous or substantially complementary to the delivered material (Zamore et al., 2000, Cell, 101:25; Fire et al., 1998, Nature, 391:806; Hamilton et al., 1999, Science, 286:950; Lin et al., 1999, Nature, 402:128; Sharp, 1999, Genes & Dev., 13:139; and Strauss, 1999, Science, 286:886). This effect has been observed broadly among animals including humans (Oelgeschlager et al., 2000; Svoboda et al., 2000; Wianny and Zernicka-Goetz, 2000; Catalanotto et al., 2000). The corresponding process is referred to as post-transcriptional gene-silencing (PTGS) in plants, and quelling in fungi (Heifetz et al., International PCT Publication No. WO 99/61631; Cogoni and Macino, 1999; Dalmay et al., 2000, Ketting and Plasterk, 2000; Mourrain et al., 2000; Smardon et al., 2000). dsRNA, or otherwise introduced transgenes, can also lead to transcriptional gene silencing by RNA-directed DNA methylation of cytosines, with targets as short as 30 base pairs methylated (Wassenegger, 2000).

Evolutionarily, PTGS and RNAi are thought to serve a cellular defense function by preventing the expression of foreign gene (Fire et al., 1999, Trends Genet., 15, 358). This mechanism can naturally protect the genome against transposons, viruses, and other mobile genetic elements. Viral infection and random transposon integration often involves the production of dsRNA, which triggers the RNAi response through an at present unknown mechanism, though distinct from that involving dsRNA-specific ribonucleases (see for example U.S. Pat. Nos. 6,107,094; 5,898,031; Clemens et al., 1997, J. Interferon & Cytokine Res., 17, 503 524; Adah et al., 2001, Curr. Med. Chem., 8, 1189). Upon the production of dsRNA, however, the cellular response does involve mRNA degradation. This defense mechanism can be overcome by expressing proteins that suppress the RNAi/PTGS mechanism (Jensen et al, 1999; Ketting et al., 1999; Ratcliff et al., 1999; Tabara et al., 1999; Lucy et al.; 2000; Voinnet et al., 2000).

When dsRNAs are in cells, the activity of dicer, a ribonuclease III enzyme, is activated (Bass, 2000, Cell, 101:235; Zamore et al., 2000, Cell, 101:25; Hammond et al., 2000, Nature, 404:293). Activated dicer promotes the processing of the dsRNA into siRNAs (Zamore et al., 2000, Cell, 101:25; Bass, 2000, Cell, 101:235; Berstein et al., 2001, Nature, 409:363). These siRNAs are typically 21-23 nucleotides, comprising ˜19 base pair duplexes (Zamore et al., 2000, Cell, 101:25; Elbashir et al., 2001, Genes Dev., 15:188). Small temporal RNAs can also be produced by dicer (Hutvagner et al., 2001, Science, 293:834). The RNAi response itself, however, involves the RNA-induced silencing complex (RISC), which has endonuclease activity, and mediates the cleavage of single stranded RNA having sequence complementary to the antisense strand of the siRNA duplex. This cleavage generally occurs in the middle of the homologous region (Elbashir et al., 2001, Genes Dev., 15:188).

RNAi can be achieved by the delivery of duplexes of synthetic 21-23 nucleotide RNAs (Elbashir et al., 2001, Nature, 411, 494 and Tuschl et al., International PCT Publication No. WO 01/75164). Such a siRNA has to have specific length, structure, composition, and sequence to initiate a desired specific activity (Elbashir et al., 2001, EMBO J., 20, 6877 and Tuschl et al., International PCT Publication No. WO 01/75164). In particular, 21 nucleotide duplexes are most active with a 3′-terminal dinucleotide overhang. A 5′-phosphate on the target-complementary strand of the siRNA is necessary for the RNAi effect, with ATP used to maintain this moiety (Nykanen et al., 2001, Cell, 107:309). Substitution of a base in one or both strands with a 2′-deoxy nucleotide, a similar substitution with a 2′O-methyl nucleotide or single mismatch sequences in the center of the duplex may abolish RNAi activity.

Longer dsRNAs can also be used to attenuate gene expression (Beach et al., International PCT Publication No. WO 01/68836). Long specific (141 base pairs-488 base pairs, as well as 550 base pairs-714 base pairs) enzymatically synthesized or vector expressed dsRNAs have been demonstrated to attenuate particular target genes (Li et al., International PCT Publication No. WO 00/44914; Zernicka-Goetz et al., International PCT Publication No. WO 01/36646). These elements can be delivered into cells to affect RNA (Fire et al., International PCT Publication No. WO 99/32619).

The therapeutic delivery of siRNA has been of great interest. The primary area of focus has been the development of methods to deliver a siRNA against a single target. These delivery systems are often polymeric, such as poly(beta-amino esters) prepared from the conjugate addition of bis(secondary amines) or primary amines to a bis(acrylate ester), or viral (see for example Anderson et al US Patent Application 20040071654; Hedley et al U.S. Pat. No. 5,783,567; Siegel et al U.S. Pat. No. 5,942,634; Engler et al U.S. Pat. No. 7,002,027; Kowalik et al US Patent Application 20060073127; Chen et al US Patent Application 20050008617; Chen et al US Patent Application 20060240554). A number of other approaches have also been described. Deschamps Depaillette et al., (International PCT Publication No. WO 99/07409) describe specific compositions consisting of particular dsRNA molecules combined with certain anti-viral agents. Roelvink et al., (US Patent Application 20050197313) describes a multiple-promoter expression cassette that can simultaneously deliver multiple siRNA agents. These agents are specifically designed to target the same genetic site using multiple siRNAs to account for variations such as SNPs. Chen et al (US Patent Application 20060040882) describes a method to deliver polypeptides with siRNA to enhance the effects of the siRNA.

Of particular note, it is well described in the art that RNAi alone is not thought likely to cure genetic diseases or viral infection clinically due to the danger of activating interferon response (Tuschl, 2001, Chem. Biochem., 2, 239-245) even though the same has been demonstrated in a Drosophila system (Tuschl et al., International PCT Publication No. WO 01/75164). The instant invention describes methods of using siRNA delivery to augment disease treatment and/or reduce predictable or other side effects.

Disease processes are typically complex and multifactoral. Many diseases are characterized by the complex interplay of various genetic, protein, lipid and polysaccharide elements. Typical drug development approaches focus on a single target thought to play an important role in a disease process. These drugs are often not completely effective as additional components of the disease etiology can maintain the pathological processes. Indeed, recent evidence has demonstrated that combinations of drugs targeting multiple components of a disease process have improved performance (see for example U.S. Pat. Nos. 7,148,216, 6,955,815, 6,897,206, 6,846,816, 6,693,125 and 6,569,853). Similarly, siRNA to a single gene can be used to sensitize cells to drugs. For example, siRNA to the ataxia telangiectasia mutated (ATM) gene, which alone increases cell cycling, sensitizes PC3 cells to doxorubicin (Mukhopadhyay et al Cancer Res 65:2872). Similarly, siRNAs have been identified that sensitize cells to cisplatin (Bartz et al, Mol Cell Biol 26:9377). Emerging concerns that RNAi monotherapies may fail to prevent or treat diseases of conditions involve concerns about mutuation and RNAi suppression with respect to viral and cellular targets. Suggestions to combine RNAi effectors or to combine multiple effectors with protein-based silencers have started to emerge, including the use of vectors engineered to deliver multiple RNAi effectors through gene replacement therapies. Lacking in the prior art is a mechanism for delivery of linked, chemically modified RNA co-interference compositions delivering a fixed ratio of double-stranded oligoribonucleotides capable of mediating RNA co-interference in a cell or an organism.

The instant invention teaches novel methods to modulate, knock down or knock-out two or more targets through a single drug entity with one or more active agents comprising siRNA. Essential to such therapeutic agents is the selection of the targets, the definition of active agents, the formulation of a delivery vehicle, the use of such entities to treat diseases.

### SUMMARY OF THE INVENTION

The instant invention describes the use of RNAi co-interference agents comprising two or more double-stranded oligoribonucleotides and other active agents to augment the therapeutic effect of another drug. Drugs typically elicit their effects through the activation or inhibition of particular pathways that are relevant to a specific process. Associated with this effect is often an activating or inhibiting effect that manifests as a potentially unwanted side effect. Many drugs have limited success as the pathways they are inhibiting are not the sole contributing factor leading to the phenotype that is to be changed. This invention recognizes these insufficiencies and teaches methods to rationally and empirically overcome them.

In this invention a “first agent” is combined with a “second agent” to achieve an improved cellular, organ and/or systemic effect. The first agent is one or more siRNAs, small molecule drugs, metabolites, sugars, polysaccharides, lipids, therapeutic peptides, therapeutic proteins (i.e. a recombinant protein, an antibody, etc), other RNAs, or DNAs. The second agent is one or more siRNAs specific to one or more genes. In a preferred embodiment, the first agent and second agent are both a siRNA specific to distinct genes. In another preferred embodiment, the second agent is at least two siRNAs specific to particular (and different) genes. In this invention, one or more first agents are combined with a second agent, yielding an improved cellular or systemic effect compared to the first agent or the second agent. In one embodiment, a second agent of one or more specific siRNA(s) is delivered with a first agent. In another embodiment, a second agent of a specific siRNA is delivered with a first agent of another siRNA. In this embodiment the two siRNAs target different genes.

This invention specifically teaches the development and application of RNA Co-Interference (RNAco-i), which is the use of two or more double-stranded oligoribonucleotides, such as siRNA, to one or more distinct targets. RNAco-i also constitutes, by definition herein, a composition wherein one or more, but not all, of the RNAi conferring agents are replaced with another active agents. Such active agents can constitute lipids, polymers, nucleic acids, small molecules, and other such substances that can confer a measurable biological response.

It is well understood by those skilled in the art that RNAi can be by a number of means including, but not limited to siRNA, short hairpin RNA (shRNA), miRNA and small activating RNAs (saRNA). RNAi effects can also be achieved using other species of nucleotides, including single stranded antisense oligonucleotides (ASO), triplex-forming nucleotides, ribozymes, DNAzymes and the like.

Previous reports (Roelvink et al., US Patent Application 20050197313) have described a system delivering multiple siRNAs to target multiple versions of the same gene. The instant invention describes co-delivery of siRNAs to more than one distinct gene. Additionally, the delivery of multiple siRNAs as such, can also be done with another non-siRNA molecule. When siRNAs are delivered with a non-siRNA molecule, the ability to deliver multiple RNAs targeting a common gene to account for genetic variability is again of interest. The invention, in particular involves co-delivery of siRNAs covalently linked by through a linking moiety and present in fixed ratios.

Combinations of first agents and second agents (one or more than one siRNAs) are chosen to improve the total cellular or systemic effect. The effect can be a therapeutic effect, wherein the output is increased. Alternatively, an effect can be a side effect, wherein the readout for the side effect would be reduced. In a preferred embodiment, based on known effects of the first agent, a siRNA is selected and/or designed to shut down a second critical pathway or to shut down a pathway whose activation leads to a side effect. One such example would be the addition of NP-siRNA (in which NP is influenza nucleocapsid protein): sense, 5′-GGAUCUUAUUUCUUCGGAGdTdT-3′; complementary strand, 3′-CCUAGAAUAAAGAAGCCUCdTdT-5′ (Ge et al PNAS 101: 8676) to augment the treatment of influenza with oseltamivir (Tamiflu®). In another embodiment, a given first agent is applied to a model system and multiple rationally selected siRNAs selected towards various targets of interest are delivered in a screen, with the maximal effect used to define the best combination of these rationally selected pathways. In this embodiment, one or more siRNAs can be co-delivered with the drug to achieve a maximal benefit. In still another embodiment, siRNAs that systematically cover all genes in a given genome can be applied along with the first agent in a screen, to search for the pair or set that achieves the maximal desired effect.

In each of these embodiments, the effect can be observed in cell models, animal models and/or human subjects. Assay and models employed are those that are associated with the phenotype(s) being probed. Preferred phenotypes and disease models are those that are not caused by a single gene deficiency such as hemophilia. Preferred diseases are those that are multigenic or involve multiple pathways, including cancer, Parkinson's disease, Alzheimer's disease, diabetes, atherosclerosis, and asthma. Cellular and systemic outputs can be performed by any assay described in the art.

In each of these embodiments the first agent and the second agent must be delivered. In a preferred embodiment, delivery is to a human subject. In other embodiments, delivery is to a cell or an animal. Delivery of the first and second agents can be either simultaneous or at distinct times. In a preferred embodiment, delivery of the first and second agents is at the same time in a common vehicle. In another preferred embodiment, the ratio between the first agent and the second agent is fixed. In these embodiments, the agents can be co-encapsulated within the same delivery vehicle, encapsulated within separate (same or distinct) delivery vehicles and subsequently combined at the desired ratio, molecularly linked, or molecularly linked and encapsulated within a delivery vehicle. The vehicles may release the active agents (siRNA or otherwise) at the same time, at distinct but controlled times, distinct but uncontrolled times, or never. Vehicles can include any method known in the art, including liposomes, poly(beta-amino esters), dextrans, PEG, PEI, atellocollagen, cyclodextrin, chitosan, and other cationic polymers. In this embodiment, the two agents can be conjugated or incorporated serially or simultaneously. In the case that the second agent is a DNA, RNA, or siRNA, virus-based delivery vehicles can also be employed.

In another embodiment, delivery of the first and second agents is at the same time in distinct vehicles. Vehicles for the agents consisting of at least one nucleic acid moiety can include any described in the art, including liposomes, poly(beta-amino esters), dextrans, PEG, PEI, atellocollagen, cyclodextrin, chitosan, other cationic polymers, viruses, etc (see for example Anderson et al US Patent Application 20040071654; Hedley et al U.S. Pat. No. 5,783,567; Siegel et al U.S. Pat. No. 5,942,634; Engler et al U.S. Pat. No. 7,002,027; Kowalik et al US Patent Application 20060073127; Chen et al US Patent Application 20050008617; Chen et al US Patent Application 20060240554). In this embodiment, only the nucleic acid comprising agent necessarily requires a delivery vehicle. In the embodiment where the first agent is not a nucleic acid, the first agent can be delivered with any delivery agent described in the art or without such a delivery agent.

In another embodiment, the first and second agents can be delivered at distinct times. Delivery vehicles for the nucleic acid comprising agents can include any described in the art, including liposomes, poly(beta-amino esters), dextrans, PEG, PEI, atellocollagen, cyclodextrin, chitosan, other cationic polymers, viruses etc (see for example Anderson et al US Patent Application 20040071654; Hedley et al U.S. Pat. No. 5,783,567; Siegel et al U.S. Pat. No. 5,942,634; Engler et al U.S. Pat. No. 7,002,027; Kowalik et al US Patent Application 20060073127; Chen et al US Patent Application 20050008617; Chen et al US Patent Application 20060240554). In this embodiment, only the nucleic acid comprising agent necessarily requires a delivery vehicle. In the embodiment where the first agent is not a nucleic acid, the first agent can be delivered with any delivery agent described in the art or without such a delivery agent. The time between the delivery of the first and second agent can be defined rationally by first principles of the kinetics, delivery, release, agent pharmacodynamics, agent pharmacokinetics or any combination thereof. Alternatively, the time between the delivery of the first and second agents can be defined empirically by experiments to define when a maximal effect can be given.

The two agents selected can alternatively be molecularly linked into a single entity. This entity must be formed such that the two agents retain function. In a preferred embodiment, the first agent is a siRNA, which is bound to a second siRNA. In this embodiment, the two siRNAs are preferentially targeted at different genes. Alternatively, they can target different genetic sequences of a common gene. In this embodiment, the second agent can be more than one siRNA, with the synthesis process used in series (or parallel) to add multiple siRNAs together into a single entity. In a preferred embodiment, two siRNAs are preferably linked through their 3′ ends, using either a 3′ or 2′ site. The linking agent can be a phosphate, a cholesterol, a therapeutic agent, an ester linker, a triacylglycerol, PEG, PEI, or dextran. Alternatively, the siRNAs can be linked through a shared 5′ phosphate. Linkages can also be made by cleavable agents, such as esters. Upon internalization through the endosome pathway, increased acidity will split the ester leading to a siRNA-aldehyde and siRNA alcohol. In this embodiment, the new composition can be delivered as is or in an agent including, but not limited to, liposomes, PEI, PEG, PLGA, PEG-PLGA, poly(beta-amino esters), and dextrans.

In another embodiment, the first agent not a siRNA can be linked to a selected siRNA. This linkage can be through any chemistry known in the art—cleavable or not. The new composition can be delivered as is or in an agent including, but not limited to, liposomes, PEI, PEG, PLGA, PEG-PLGA, poly(beta-amino esters), and dextrans.

In one embodiment, the invention provides an RNA co-interference composition comprising: (a) a first region of contiguous ribonucleotides defining a first double-stranded oligoribonucleotide complementary to a hybridization sequence of a target nucleic acid, said first oligoribonucleotide having at least one functional group; (b) a second region of contiguous ribonucleotides defining a second double-stranded oligoribonucleotide complementary to a hybridization sequence of said target nucleic acid, said second oligoribonucleotide having at least one functional group; and (c) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; wherein said first oligoribonucleotide and said second oligoribonucleotide are joined by said linking moiety through said functional groups of said first oligoribonucleotide and said second oligoribonucleotide with said reactive groups of said linking moiety, and wherein said RNA co-interference composition is capable of modulating expression of said target nucleic acid through RNA co-interference.

In another embodiment, the invention provides an RNA co-interference composition comprising: (a) a first region of contiguous ribonucleotides defining a first double-stranded oligoribonucleotide complementary to a hybridization sequence of a first target nucleic acid, said first oligoribonucleotide having at least one functional group; (b) a second region of contiguous ribonucleotides defining a second double-stranded oligoribonucleotide complementary to a hybridization sequence of a second target nucleic acid, said second oligoribonucleotide having at least one functional group; and (c) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; wherein said first oligoribonucleotide and said second oligoribonucleotide are joined by said linking moiety through said functional groups of said first oligoribonucleotide and said second oligoribonucleotide with said reactive groups of said linking moiety, and wherein said RNA co-interference composition is capable of modulating expression of said first target nucleic acid and said second target nucleic acid through RNA co-interference.

In another embodiment, the invention provides an RNA co-interference composition comprising: (a) a first region of contiguous ribonucleotides defining a first double-stranded oligoribonucleotide complementary to a first hybridization sequence of a first target nucleic acid, said first oligoribonucleotide having at least one functional group; (b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group; (c) a third region of contiguous ribonucleotides defining a third double-stranded oligoribonucleotide complementary to a hybridization sequence of any of the foregoing target nucleic acids, or to a hybridization sequence of a different target nucleic acid said third oligoribonucleotide having at least one functional group; (d) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; wherein said first oligoribonucleotide, said second oligoribonucleotide, and said third oligoribonucleotide are joined by said linking moiety through said functional groups of said first oligoribonucleotide, said second oligoribonucleotide, and said third oligoribonucleotide with said reactive groups of said linking moiety, and wherein said RNA co-interference composition is capable of modulating expression of said target nucleic acid through RNA co-interference.

In another embodiment, the invention provides an RNA co-interference composition comprising: (a) a first region of contiguous ribonucleotides defining a first double-stranded oligoribonucleotide complementary to a first hybridization sequence of a first target nucleic acid, said first oligoribonucleotide having at least one functional group; (b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group; (c) a third region of contiguous ribonucleotides defining a third double-stranded oligoribonucleotide complementary to a hybridization sequence of any of the foregoing target nucleic acids, or to a hybridization sequence of a different target nucleic acid said third oligoribonucleotide having at least one functional group; (d) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and (e) a second linking moiety having at least two reactive groups reactive with two or more functional groups corresponding and reactive with said second linking moiety, wherein said first oligoribonucleotide, said second oligoribonucleotide, and said third oligoribonucleotide are joined by said first linking moiety and said second linking moiety through said functional groups of said first oligoribonucleotide, said second oligoribonucleotide, and said third oligoribonucleotide with said reactive groups of said first linking moiety and said second linking moiety, and wherein said RNA co-interference composition is capable of modulating expression of said target nucleic acids to which said first oligoribonucleotide, said second oligoribonucleotide, and said third oligoribonucleotide are complementary through RNA co-interference.

In another embodiment, the invention provides an RNA co-interference composition comprising: (a) a first region of contiguous ribonucleotides defining a first double-stranded oligoribonucleotide complementary to a hybridization sequence of a first target nucleic acid, said first oligoribonucleotide having at least one functional group; (b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group; (c) a third region of contiguous ribonucleotides defining a third double-stranded oligoribonucleotide complementary to a hybridization sequence of any of the foregoing target nucleic acids, or to a hybridization sequence of a different target nucleic acid; (d) a fourth region of contiguous ribonucleotides defining a fourth double-stranded oligoribonucleotide complementary to hybridization sequence of any of the foregoing target nucleic acids, or to a hybridization sequence of a different target nucleic acid, said fourth oligoribonucleotide having at least one functional group; (d) one or more linking moieties that are the same or different comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; wherein said first oligoribonucleotide, said second oligoribonucleotide, said third oligoribonucleotide and said fourth oligoribonucleotide are joined by said linking moiety through said functional groups of said first oligoribonucleotide, said second oligoribonucleotide, said third oligoribonucleotide and said fourth oligoribonucleotide with said reactive groups of said linking moiety, and wherein said RNA co-interference composition is capable of modulating expression of said target nucleic acids to which said first oligoribonucleotide, said second oligoribonucleotide, said third oligoribonucleotide and said fourth oligoribonucleotide are complementary through RNA co-interference.

In another embodiment, the invention provides an RNA co-interference composition comprising: (a) a first region of contiguous ribonucleotides defining a first double-stranded oligoribonucleotide complementary to a hybridization sequence of a first target nucleic acid, said first oligoribonucleotide having at least one functional group; (b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group; (c) a third region of contiguous ribonucleotides defining a third double-stranded oligoribonucleotide complementary to a hybridization sequence of any of the foregoing target nucleic acids, or to a hybridization sequence of a different target nucleic acid; (d) a fourth region of contiguous ribonucleotides defining a fourth double-stranded oligoribonucleotide complementary to a hybridization sequence of any of the foregoing target nucleic acids, or to a hybridization sequence of a different target nucleic acid, said fourth oligoribonucleotide having at least one functional group; (d) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and (e) one or more additional linking moieties that are the same or different having at least two reactive groups reactive with two or more functional groups corresponding and reactive with said additional linking moieties, wherein said first oligoribonucleotide, said second oligoribonucleotide, said third oligoribonucleotide and said fourth oligoribonucleotide are joined by said first linking moiety and said additional linking moieties through said functional groups of said first oligoribonucleotide, said second oligoribonucleotide, said third oligoribonucleotide and said fourth oligoribonucleotide with said reactive groups of said first linking moiety and said additional linking moieties, and wherein said RNA co-interference composition is capable of modulating expression of said target nucleic acids to which said first oligoribonucleotide, said second oligoribonucleotide, said third oligoribonucleotide and said fourth oligoribonucleotide are complementary through RNA co-interference.

In one aspect of the embodiments, the invention provides an RNA co-interference composition, wherein the oligoribonucleotides of said RNA co-interference composition comprise a sense and anti-sense strand, wherein the anti-sense strand has a sequence sufficiently complementary to a target nucleic acid sequence to direct target specific RNA co-interference and wherein the sense strand or anti-sense strand is modified by the substitution of at least one internal ribonucleotide with a modified ribonucleotide.

In another aspect, the invention provides an RNA co-interference composition, wherein the hydrophilic polymer is a co-block polymer.

In another aspect, the invention provides an RNA co-interference composition, further comprising an additional conjugating linker for linking a conjugate moiety to at least one of the oligoribonucleotides.

In another aspect, the invention provides an RNA co-interference composition, wherein the conjugate moiety is selected from the group consisting of: a sugar, a polysaccharide, a lipid, RNA, DNA, aromatic and non-aromatic lipophilic molecules including steroid molecules, proteins including antibodies, enzymes, and serum proteins, peptides, water-soluble and lipidsoluble vitamins, water-soluble and lipid-soluble polymers, small molecules including drugs, toxins, reporter molecules, and receptor ligands, a metabolite, carbohydrate complexes, nucleic acid cleaving complexes, metal chelators including porphyrins, texaphyrins, and crown ethers, intercalators including hybrid photonucleaselintercalators and photoactive and redox active crosslinking agents.

In another aspect, the invention provides an RNA co-interference composition, wherein said RNA co-interference composition has enhanced in vivo stability as compared to the corresponding unmodified oligoribonucleotides.

In another aspect, the invention provides an RNA co-interference composition, wherein said RNA co-interference composition has enhanced target efficacy as compared to the corresponding unmodified oligoribonucleotides.

In another aspect, the invention provides an RNA co-interference composition, wherein said RNA co-interference composition has enhanced cellular penetration as compared to the corresponding unmodified oligoribonucleotides.

In another aspect, the invention provides an RNA co-interference composition, wherein at least one of the modified ribonucleotides is a sugar-modified ribonucleotide.

In another aspect, the invention provides an RNA co-interference composition, wherein at least one of the modified ribonucleotides is a nucleobase-modified ribonucleotide.

In another aspect, the invention provides an RNA co-interference composition, wherein a target sequence specifies an amino acid sequence of a cellular protein.

In another aspect, the invention provides an RNA co-interference composition, wherein a target sequence specifies an amino acid sequence of a viral protein.

In another aspect, the invention provides an RNA co-interference composition, wherein the modified ribonucleotide is selected from the group consisting of 2′-deoxy ribonucleotide, 2′-fluoro ribonucleotide, 2′-deoxy-2′-fluoro, 2′-amino ribonucleotide, 2′-O-methyl ribonucleotide, 2′-O-(2-methoxyethyl), and 2′-thio ribonucleotide.

In another aspect, the invention provides an RNA co-interference composition, wherein the modified ribonucleotide is a 2′-deoxy ribonucleotide.

In another aspect, the invention provides an RNA co-interference composition, wherein the modified ribonucleotide is in the sense strand.

In another aspect, the invention provides an RNA co-interference composition, wherein the modified ribonucleotide is in the anti-sense strand.

In another aspect, the invention provides an RNA co-interference composition, wherein the modified ribonucleotides are in the sense and anti-sense strands.

In another aspect, the invention provides an RNA co-interference composition, wherein the modified ribonucleotide is selected from the group consisting of 2′-fluoro cytidine, 2′-fluoro-uridine, 2′-fluoro adenosine, 2-fluoro guanosine, 2′-amino cytidine, 2′-amino adenosine, 1′ amino guanosine and 2′-amino-butyryl-pyrene uridine.

In another aspect, the invention provides an RNA co-interference composition, wherein the modified ribonucleotide is selected from the group consisting of 5-bromo-uridine, 5-iodo-uridine, 5-methyl-cytidine, ribo-thymidine, 2-aminoopurine, 4-thio-uridine and 5-amino-allyl-uridine.

In another aspect, the invention provides an RNA co-interference composition, wherein the modified ribonucleotide is a back-bone modified ribonucleotide.

In another aspect, the invention provides an RNA co-interference composition, wherein the modified ribonucleotide contains a phosphorothiolate group.

In another aspect, the invention provides an RNA co-interference composition, wherein a 3′-OH terminus of the sense strand or anti-sense strand is modified.

In another aspect, the invention provides an RNA co-interference composition, wherein the oligonucleotides are between about 10 to 50 residues in length.

In another aspect, the invention provides an RNA co-interference composition, wherein the oligonucleotides are between about 15 to 45 residues in length.

In another aspect, the invention provides an RNA co-interference composition, wherein the oligonucleotides are between about 20 to 40 residues in length.

In another aspect, the invention provides an RNA co-interference composition, wherein the oligonucleotides are between about 19 to 25 residues in length.

In another aspect, the invention provides an RNA co-interference composition, wherein the oligonucleotides are between about 19 to 22 residues in length.

In another aspect, the invention provides an RNA co-interference composition, wherein the oligonucleotides are between about 21 to 22 residues in length.

In another aspect, the invention provides an RNA co-interference composition, wherein the oligonucleotides are between about 27 to 29 residues in length.

In another aspect, the invention provides an RNA co-interference composition, wherein the oligonucleotides are chemically synthesized.

In another aspect, the invention provides an RNA co-interference composition, wherein the composition has a net positive charge.

In another aspect, the invention provides an RNA co-interference composition, wherein the composition has a positive zeta potential.

In another aspect, the invention provides an RNA co-interference composition and a pharmaceutically acceptable carrier.

In another embodiment, the invention provides a method of activating target-specific RNA co-interference in a cell comprising introducing into said cell an RNA co-interference composition, said RNA co-interference composition being introduced in an amount sufficient for modulation of a target nucleic acid to occur, thereby activating target specific RNA co-interference in the cell.

In one aspect of this embodiment, the invention provides a method, wherein the RNA co-interference composition is introduced into the cell by contacting the cell with the RNA co-interference composition.

In another aspect, the invention provides a method, wherein the RNA co-interference composition is introduced into the cell by contacting the cell with a composition comprising the RNA co-interference composition and a lipophilic carrier.

In another aspect, the invention provides a method, wherein the target nucleic acid specifies the amino acid sequence of a protein involved in or predicted to be involved in a human disease, disorder, condition or trait.

In one embodiment, the invention provides a method of activating a target-specific RNA co-interference in an organism comprising administering to said organism the RNA co-interference composition, said RNA co-interference composition being administered in an amount sufficient for modulation of a target nucleic acid to occur, thereby activating target specific RNA co-interference in the organism.

In one aspect of this embodiment, the invention provides a method, wherein the RNA co-interference composition is administered by intravenous, intramuscular, subcutaneous, or intraperitoneal injection, topical application, local infusion, or oral administration.

In another aspect, the invention provides a method, wherein the target nuclei acid specifies the amino acid sequence of a protein involved in or predicted to be involved in a human disease, disorder, condition or trait.

In another aspect, the invention provides a method, wherein modulation of the target nucleic acid produces a loss-of-function phenotype.

In another aspect, the invention provides a method, wherein modulation of the target nucleic acid sequence corresponds to a decrease of at least 10 percent of the protein specified by said target nucleic acid.

In another aspect, the invention provides a method, wherein modulation of the target nucleic acid sequence corresponds to a decrease of at least 25 percent of the protein specified by said target nucleic acid.

In another aspect, the invention provides a method, wherein modulation of the target nucleic acid sequence corresponds to a decrease of at least 50 percent of the protein specified by said target nucleic acid.

In another aspect, the invention provides a method, wherein modulation of the target nucleic acid sequence corresponds to a decrease of at least 75 percent of the protein specified by said target nucleic acid.

In another aspect, the invention provides a method, wherein modulation of the target nucleic acid sequence corresponds to a decrease of at least 90 percent of the protein specified by said target nucleic acid.

In one embodiment, the invention provides a method of evaluating gene function in a cell or an organism, comprising: (a) introducing into said cell or organism the RNA co-interference composition of any of the preceding claims; (b) maintaining the cell or organism under conditions allowing target-specific RNA co-interference to occur; (c) determining a characteristic or property of said cell or said organism; and (d) comparing said characteristic or property to a suitable control, the comparison yielding information about the function of the gene.

In one embodiment, the invention provides a method of validating a candidate protein as a suitable target for drug discovery, comprising: (a) introducing into a cell or organism RNA co-interference composition of any of the preceding claims; (b) maintaining the cell or organism under conditions allowing target-specific RNA co-interference to occur; (c) determining a characteristic or property of said cell or said organism; and (d) comparing said characteristic or property to a suitable control, the comparison yielding information about whether the candidate protein is a suitable target for drug discovery.

In one embodiment, the invention provides a method of validating a candidate RNA co-interference composition as a suitable composition for drug therapy, comprising: (a) introducing into a cell or organism an RNA co-interference composition; (b) maintaining the cell or organism under conditions allowing target-specific RNA co-interference to occur; (c) determining a characteristic or property of said cell or said organism; and (d) comparing said characteristic or property to a suitable control, the comparison yielding information about whether the candidate RNA co-interference composition is a suitable target for drug therapy.

In one embodiment, the invention provides a kit comprising reagents for activating target-specific RNA co-interference in a cell or organism, said kit comprising: (a) one or more RNA co-interference compositions; and (b) instructions for use.

In one embodiment, the invention provides a method of treating a disease, disorder, condition or trait associated with the activity of a protein specified by a target nucleic acid in a subject, comprising administering to said subject an RNA co-interference composition, said RNA co-interference composition being administered in an amount sufficient for modulation of the target nucleic acid to occur, thereby treating the disease, disorder, condition or trait associated with the protein.

In one aspect, the invention provides an RNA co-interference composition, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by at least 80% sequence identity of said anti-sense strand and said target nucleic acid.

In another aspect, sufficient complementarity is achieved by at least 90% sequence identity of said anti-sense strand and said target nucleic acid.

In another aspect sufficient complementarity is achieved by at least 91% sequence identity of said anti-sense strand and said target nucleic acid.

In another aspect sufficient complementarity is achieved by 92% sequence identity of said anti-sense strand and said target nucleic acid.

In another aspect sufficient complementarity is achieved by 93% sequence identity of said anti-sense strand and said target nucleic acid.

In another aspect sufficient complementarity is achieved by 94% sequence identity of said anti-sense strand and said target nucleic acid.

In another aspect sufficient complementarity is achieved by 95% sequence identity of said anti-sense strand and said target nucleic acid.

In another aspect sufficient complementarity is achieved by 96% sequence identity of said anti-sense strand and said target nucleic acid.

In another aspect sufficient complementarity is achieved by 97% sequence identity of said anti-sense strand and said target nucleic acid.

In another aspect sufficient complementarity is achieved by 98% sequence identity of said anti-sense strand and said target nucleic acid.

In another aspect sufficient complementarity is achieved by 99% sequence identity of said anti-sense strand and said target nucleic acid.

In another aspect sufficient complementarity is achieved by 100% sequence identity of said anti-sense strand and said target nucleic acid.

In one aspect, the invention provides an RNA co-interference composition, wherein the composition comprises a fixed ratio of double-stranded oligoribonucleotides.

In one aspect, the invention provides an RNA co-interference composition, wherein the composition synergistically modulates expression of one or more target nucleic acids through RNA co-interference.

In one embodiment, the invention provides an RNA co-interference composition having the structural formula:

A-L1-L2-B, wherein (a) A is a double-stranded oligoribonucleotide complementary to a first hybridization sequence of a first target nucleic acid; (b) L1 is a first linking moiety comprising a non-biologically active strand of RNA or DNA capable of being cleaved endogenously, thereby releasing said oligoribonucleotide from said first linking moiety L1, said first linking moiety having at least one functional group; (c) L2 is a second linking moiety capable of covalently bonding to linker L1 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and (d) B is a second double-stranded oligoribonucleotide complementary to a hybridization sequence of one or more target nucleic acids, wherein said hybridization sequence is (i) the same or different to said first hybridization sequence of said first target nucleic acid; or (ii) a hybridization sequence to a second target nucleic acid, said second oligoribonucleotide having at least one functional group, wherein said second oligoribonucleotide is capable of being joined to said second linking moiety L2 through interaction of said functional group and said reactive group; and wherein said RNA co-interference composition is capable of modulating expression of said one or more target nucleic acids through RNA co-interference.

In another embodiment, the invention provides an RNA co-interference composition having the structural formula:

A-L-B,

wherein (a) A is a double-stranded oligoribonucleotide complementary to a first hybridization sequence of a first target nucleic acid, said first oligoribonucleotide having at least one functional group; (b) L is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and (c) B is a second double-stranded oligoribonucleotide complementary to a hybridization sequence of a second target nucleic acids, wherein said hybridization sequence is (i) the same or different to said first hybridization sequence of said first target nucleic acid; or (ii) a hybridization sequence to a second target nucleic acid, said second oligoribonucleotide having at least one functional group, wherein said first and said second oligoribonucleotides are capable of being joined by said linking moiety L through interaction of said functional groups and said reactive groups; and wherein said RNA co-interference composition is capable of modulating expression of said first or said first and said second target nucleic acids through RNA co-interference.

In another embodiment, the invention provides an RNA co-interference composition having the structural formula:

A-L1-L2-X,

wherein (a) A is a double-stranded oligoribonucleotide complementary to a first hybridization sequence of a first target nucleic acid; (b) L1 is a first linking moiety comprising a non-biologically active strand of RNA or DNA capable of being cleaved endogenously, thereby releasing said oligoribonucleotide from said first linking moiety, said first linking moiety having at least one functional group; (c) L2 is a second linking moiety capable of bonding to linker L1 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide, and having one or more one reactive groups; and (d) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups, wherein said one or more oligoribonucleotides is capable of being joined to said second linking moiety L2 through interaction of said one or more functional groups and said one or more reactive groups; and wherein said RNA co-interference composition is capable of modulating expression of said one or more target nucleic acids through RNA co-interference.

In another embodiment, the invention provides an RNA co-interference composition having the structural formula:

A-L-X, wherein (a) A is a double-stranded oligoribonucleotide complementary to a first hybridization sequence of a first target nucleic acid; (b) L is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and (c) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups, wherein said one or more oligoribonucleotides is capable of being joined to said linking moiety L through interaction of said one or more functional groups and said one or more reactive groups; and wherein said RNA co-interference composition is capable of modulating expression of said one or more target nucleic acids through RNA co-interference.

In another embodiment, the invention provides an RNA co-interference polymeric composition having the structural formula:

A-[L-X]n, wherein (a) A is a double-stranded oligoribonucleotide complementary to a first hybridization sequence of a first target nucleic acid; (b) L is a linking moiety capable of covalently bonding to two or more oligoribonucleotides, comprising the same or different branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and (c) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups, wherein n is the integer 1 to about 500, and wherein said one or more oligoribonucleotides X are capable of being joined to said linking moiety L through interaction of said one or more functional groups and said one or more reactive groups; wherein said joined linking moiety L and said one or more oligoribonucleotides X comprise repeating branched or unbranched monomeric units of said RNA co-interference polymeric composition wherein said RNA co-interference composition is capable of modulating expression of said one or more target nucleic acids through RNA co-interference.

In another embodiment, the invention provides an RNA co-interference composition having the structural formula:

A-L1-L2-[L3-X]n,

wherein (a) A is a double-stranded oligoribonucleotide complementary to a first hybridization sequence of a first target nucleic acid; (b) L1 is a first linking moiety comprising a non-biologically active strand of RNA or DNA capable of being cleaved endogenously, thereby releasing said oligoribonucleotide A from said first linking moiety L1, said first linking moiety having at least one functional group; (c) L2 is a second linking moiety capable of bonding linkers L1 and L3 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide, and having one or more one reactive groups; and (d) L3 is a third linking moiety the same or different to said first linking moiety L1, wherein said third linking moiety comprises a non-biologically active strand of RNA or DNA capable of being cleaved endogenously, thereby releasing said one or more oligoribonucleotide X from said third linking moiety L3, said third linking moiety having at least one functional group; (e) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups, wherein n is the integer 1 to about 500, and wherein said one or more oligoribonucleotides X are capable of being joined to said second linking moiety L2 through interaction of said one or more functional groups and said one or more reactive groups; wherein said RNA co-interference composition is capable of modulating expression of said one or more target nucleic acids through RNA co-interference.

In one embodiment, the invention provides an RNA co-interference composition having the structural formula:

A-L1-L2-[L3n₁-X]n₂,

wherein (a) A is a double-stranded oligoribonucleotide complementary to a first hybridization sequence of a first target nucleic acid; (b) L1 is a first linking moiety comprising a non-biologically active strand of RNA or DNA capable of being cleaved endogenously, thereby releasing said oligoribonucleotide A from said first linking moiety L1, said first linking moiety having at least one functional group; (c) L2 is a second linking moiety capable of bonding linkers L1 and L3 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide, and having one or more one reactive groups; (d) L3 is a third linking moiety the same or different to said first linking moiety L1, wherein said third linking moiety comprises a non-biologically active strand of RNA or DNA capable of being cleaved endogenously, thereby releasing said one or more oligoribonucleotide X from said third linking moiety L3, said third linking moiety having at least one functional group; and (e) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups, wherein n, is the integer 0 or 1, wherein n₂ is the integer 1 to about 500, wherein X may optionally contain a reactive group when n₁=0, wherein such oligoribonucleotide X having a reactive group is capable of being joined to another oligoribonucleotide X having one or more functional groups through interaction of said reactive group with said one or more functional groups of said another oligoribonucleotide X, wherein said one or more oligoribonucleotides X are capable of being joined to said second linking moiety L2 through interaction of said one or more functional groups and said one or more reactive groups; wherein said RNA co-interference composition is capable of modulating expression of said one or more target nucleic acids through RNA co-interference.

In one aspect of these embodiments, the invention provides an RNA co-interference composition, wherein the oligoribonucleotides of said RNA co-interference composition comprise a sense and an anti-sense strand, wherein the anti-sense strand has a sequence sufficiently complementary to a target nucleic acid to direct target specific RNA co-interference and wherein the sense strand or the anti-sense strand is modified by substitution of at least one internal ribonucleotide with a modified ribonucleotide.

In another aspect, the invention provides an RNA co-interference composition, further comprising an additional conjugating linker for linking a conjugate moiety to at least one of the oligoribonucleotides.

In another aspect, the invention provides an RNA co-interference composition, wherein the conjugate moiety is selected from the group consisting of: a sugar, a polysaccharide, a lipid, RNA, DNA, aromatic and non-aromatic lipophilic molecules including steroid molecules, proteins including antibodies, enzymes, and serum proteins, peptides, water-soluble and lipidsoluble vitamins, water-soluble and lipid-soluble polymers, small molecules including drugs, toxins, reporter molecules, and receptor ligands, a metabolite, carbohydrate complexes, nucleic acid cleaving complexes, metal chelators including porphyrins, texaphyrins, and crown ethers, intercalators including hybrid photonucleaselintercalators and photoactive and redox active crosslinking agents.

In another aspect, the invention provides an RNA co-interference composition, wherein at least one of the double-stranded oligoribonucleotide is selected from the group consisting of siRNA, microRNA and short hairpin RNA, and mixtures of (a), (b) and (c).

In another aspect, the invention provides an RNA co-interference composition, wherein each double-stranded oligoribonucleotide is a microRNA.

In another aspect, the invention provides an RNA co-interference composition, wherein each double-stranded oligoribonucleotide is a siRNA.

In another aspect, the invention provides an RNA co-interference composition, wherein each double-stranded oligoribonucleotide is a short hairpin RNA.

In another aspect, the invention provides an RNA co-interference composition, wherein at least one of the modified ribonucleotides is a sugar-modified ribonucleotide.

In another aspect, the invention provides an RNA co-interference composition, wherein at least one of the modified ribonucleotides is a nucleobase-modified ribonucleotide.

In another aspect, the invention provides an RNA co-interference composition, wherein at least one of the modified ribonucleotides is in the sense strand.

In another aspect, the invention provides an RNA co-interference composition, wherein at least one of the modified ribonucleotides is in the anti-sense strand.

In another aspect, the invention provides an RNA co-interference composition, wherein the modified ribonucleotides are in the sense and anti-sense strands.

In another aspect, the invention provides an RNA co-interference composition, wherein at least one of the modified ribonucleotides is a back-bone modified ribonucleotide.

In another aspect, the invention provides an RNA co-interference composition, wherein at least one of the modified ribonucleotides contains a phosphorothiolate group.

In another aspect, the invention provides an RNA co-interference composition, wherein a 3′-OH terminus of the sense strand or anti-sense strand of at least one of the oligoribonucleotides is modified.

In another aspect, the invention provides an RNA co-interference composition, wherein at least one of the modified ribonucleotides is a 2′-deoxy modified ribonucleotide.

In another aspect, the invention provides an RNA co-interference composition, wherein the oligonucleotides are between about 10 to 50 residues in length.

In another aspect, the oligonucleotides are between about 15 to 45 residues in length.

In another aspect, the oligonucleotides are between about 20 to 40 residues in length.

In another aspect, the oligonucleotides are between about 19 to 25 residues in length.

In another aspect, the oligonucleotides are between about 19 to 22 residues in length.

In another aspect, the oligonucleotides are between about 21 to 22 residues in length.

In another aspect, the oligonucleotides are between about 25 to 27 residues in length.

In another aspect, the invention provides an RNA co-interference composition, wherein the oligonucleotides are chemically synthesized.

In one embodiment, the invention provides a composition comprising an RNA co-interference composition and a pharmaceutically acceptable carrier.

In another aspect, the invention provides an RNA co-interference composition, wherein the composition is capable of synergistically modulating expression of one or more target nucleic acids through RNA co-interference.

In another aspect, the invention provides an RNA co-interference composition, wherein the hydrophilic polymer is a co-block polymer.

In another aspect, the invention provides an RNA co-interference composition, wherein the composition comprises a fixed ratio of double-stranded oligoribonucleotides.

In another aspect, the invention provides an RNA co-interference composition, wherein a first linking moiety comprises single-stranded RNA or single-stranded DNA.

In another aspect, the invention provides an RNA co-interference composition, wherein a first linking moiety comprises double-stranded RNA or double-stranded DNA.

In another aspect, the invention provides an RNA co-interference composition, wherein a first linking moiety comprises partially single stranded RNA or partially single stranded DNA.

In another aspect, the invention provides an RNA co-interference composition, wherein a third linking moiety is selected from the group consisting of single-stranded RNA, single-stranded DNA, double-stranded RNA, double-stranded DNA, partially single stranded RNA and partially single stranded DNA.

In another aspect, the invention provides an RNA co-interference composition, wherein the integer n=1 to about 250.

In another aspect, the invention provides an RNA co-interference composition, wherein the integer n=1 to about 125.

In another aspect, the invention provides an RNA co-interference composition, wherein the integer n=1 to about 100.

In another aspect, the invention provides an RNA co-interference composition, wherein the integer n=1 to about 12.

In another aspect, the invention provides an RNA co-interference composition, wherein the integer n=1 to about 10.

In another aspect, the invention provides an RNA co-interference composition, wherein the integer n=1 to about 8.

In another aspect, the invention provides an RNA co-interference composition, wherein the integer n=1 to about 6.

In another aspect, the invention provides an RNA co-interference composition, wherein the integer n=1 to about 5.

In another aspect, the invention provides an RNA co-interference composition, wherein the integer n=1 to about 4.

In another aspect, the invention provides an RNA co-interference composition, wherein the integer n=1 to about 3.

In another aspect, the invention provides an RNA co-interference composition, wherein the integer n=1 to 2.

In another aspect, the invention provides an RNA co-interference composition, wherein the integer n=1.

In one embodiment, the invention provides a method of evaluating gene function in a cell, population of cells, or an organism, comprising: (a) introducing into said cell, said population of cells or said organism one or more RNA co-interference compositions; (b) maintaining said cell, said population of cells or said organism under conditions allowing target-specific RNA co-interference to occur; (c) determining a characteristic, property or phenotype of said cell, said population of cells or said organism; and (d) comparing said characteristic, property or phenotype to a suitable control, the comparison yielding information about the function of the gene.

In one aspect of this embodiment, the invention provides a method, wherein the oligoribonucleotides of the RNA co-interference composition are selected in a manner capable of predicting synergistic modulation of said gene function.

In another embodiment, the invention provides a method of validating a candidate protein as a suitable target for drug discovery, comprising: (a) introducing into a cell, a population of cells or an organism one or more RNA co-interference compositions; (b) maintaining said cell, said population of cells or said organism under conditions allowing target-specific RNA co-interference to occur; (c) determining a characteristic, property or phenotype of said cell, said population of cells or said organism; and (d) comparing said characteristic, property or phenotype to a suitable control, the comparison yielding information about whether the candidate protein is a suitable target for drug discovery.

In another embodiment, the invention provides a method of designing a RNA co-interference composition, comprising: (a) specifying a phenotype of interest associated with a disease, disorder, condition or trait affecting a cell, population of cells or organism; (b) specifying a biochemical network of such cell, such population of cells or such organism postulated to be correlated to said specified phenotype; (c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations; (d) optimizing said simulated biochemical network by determining and constraining the values of said quantitative parameters of said interrelationships; (e) solving said mathematical equations to identify interrelationships likely to cause the transition of said cell, said population of cells or said organism from said phenotype to another phenotype, thereby identifying one or more nucleic acids associated with said phenotypic change; (f) preparing two or more double-stranded oligoribonucleotides complementary to said nucleic acids identified in step (e) capable of modulating expression of said nucleic acids through RNA co-interference; and (g) preparing an RNA co-interference composition comprising said nucleic acids prepared in accordance with step (f).

In another embodiment, the invention provides a method of designing an RNA co-interference composition, comprising: (a) specifying a phenotype of interest associated with a disease, disorder, condition or trait affecting a cell, population of cells or organism; (b) specifying a biochemical network of such cell, such population of cells or such organism postulated to be correlated to such specified phenotype; (c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations; (d) solving said mathematical equations to identify interrelationships likely to cause the transition of said cell, said population of cells or said organism from said phenotype to another phenotype, thereby identifying one or more nucleic acids associated with said phenotypic change; (e) preparing two or more double-stranded oligoribonucleotides complementary to said nucleic acids identified in step (d) capable of modulating expression of said nucleic acids through RNA co-interference; and (f) preparing an RNA co-interference composition comprising said nucleic acids prepared in accordance with step (e).

In another embodiment, the invention provides a method of designing a RNA co-interference composition, comprising: (a) specifying a phenotype of interest associated with a disease, disorder, condition or trait affecting a cell, population of cells or organism; (b) specifying a biochemical network of such cell, such population of cells or such organism postulated to be correlated to such specified phenotype; (c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations; (d) inferring additional biochemical pathways and nucleic acid targets in said cellular biochemical network by importing data into said mathematical equations; (e) optimizing said simulated biochemical network by determining and constraining the values of said quantitative parameters and said imported data of said interrelationships; (f) solving said mathematical equations to identify interrelationships likely to cause the transition of said cell, said population of cells or said organism from said phenotype to a second phenotype, thereby identifying one or more nucleic acids associated with said transition to said second phenotype; (g) preparing two or more double-stranded oligoribonucleotides complementary to said nucleic acids identified in step (f) capable of modulating expression of said nucleic acids through RNA co-interference; and (h) preparing an RNA co-interference composition comprising said nucleic acids prepared in accordance with step (g).

In another embodiment, the invention provides a method of designing a composition to modulate the expression of one or more target nucleic acids through RNA interference, comprising: (a) specifying a phenotype of interest associated with a disease, disorder, condition or trait affecting a cell, population of cells or organism; (b) specifying a biochemical network of such cell, such population of cells or such organism postulated to be correlated to such specified phenotype; (c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations; (d) inferring additional biochemical pathways and nucleic acid targets in said cellular biochemical network by importing data into said mathematical equations; (e) optimizing said simulated biochemical network by determining and constraining the values of said quantitative parameters and said imported data of said interrelationships; (f) solving said mathematical equations to identify interrelationships likely to cause the transition of said cell, said population of cells or said organism from said phenotype to a second phenotype, thereby identifying one or more nucleic acids associated with said transition to said second phenotype; (g) preparing two or more double-stranded oligoribonucleotides complementary to said nucleic acids identified in step (f) capable of modulating expression of said nucleic acids through RNA co-interference; and (h) preparing a composition comprising the double-stranded oligoribonucleotides prepared in accordance with step (g).

In one aspect of these embodiments, the invention provides a method, wherein qualitative parameters of interrelationships are set forth in mathematical equations in addition to quantitative parameters.

In another aspect, the invention provides a method, wherein the imported data is in silico data.

In another aspect, the invention provides a method, wherein the imported data is in vitro data.

In another aspect, the invention provides a method, wherein the imported data is in vivo data.

In another aspect, the invention provides a method, wherein a synergistic interrelationship likely to cause transition of said cell, said population of cells or said organism from said phenotype to a second phenotype is identified.

In another aspect, the invention provides a method, wherein the synergistic interrelationship involves a change in cellular behavior.

In another aspect, the invention provides a method, wherein the synergistic interrelationship involves is measured by a change in quantitative assay measurements of a one or more proteins or nucleic acids, or a cellular network.

In another aspect, the invention provides a method, wherein the phenotypic change occurs in a cell.

In another aspect, the invention provides a method, wherein the phenotypic change occurs in an organ.

In another aspect, the invention provides a method, wherein the phenotypic change occurs in an organism.

In another aspect, the invention provides a method, wherein the phenotypic change occurs in a cellular system.

In another aspect, the invention provides a method, wherein the phenotypic change results in modulation of expression of one or more proteins.

In another aspect, the invention provides a method, wherein the phenotypic change results in a change in protein activity.

In another aspect, the invention provides a method, wherein the synergistic change results in a change in protein activity.

In another aspect, the invention provides a method, wherein one or more mathematical equations are selected from the group consisting of: Institute for Systems Biology Measurement Approach, Genstruct Causal Modeling, Collins Mathematical Modeling, Entelos Mathematical Modeling, MNI#1, MNI#2, EQ1, EQ2, EQ3, EQ4, EQ5, EQ6, EQ7, EQ8, EQ9 and EQ10.

In a preferred embodiment, the ribonucleotides are linked through a chemical linkage to form a multifunctional siRNA. A first siRNA may include an N-hydroxysuccinimide ester (NHS-ester), an isocyanate, a nitrophenyl carbonate or an aldehyde which can be coupled to an amine group on the second siRNA. Alternatively or in addition, the first siRNA may include a maleimide, an acrylate, a vinylsulfone, an orthopyridyl-disulfide or an iodoacetamine group which can be coupled with a thiol on the second siRNA. Alternatively or in addition, a carboxylic acid group on the first siRNA can be coupled to a hydroxyl group on the second siRNA to generate an ester bond via an acid bromide or acid chloride intermediate using phosphorus tribromide or thionyl chloride, respectively.

In one embodiment, a first siRNA is an amine-modified siRNA, capable of reacting with a functional group on one or more ends of a linear or branched polymer. In one aspect of the embodiment, a second siRNA is conjugated to a NHS-ester. The NHS-ester reacts with the free amine to form a stable amide bond at pH 7-9. The NHS-ester can be optionally joined to the polymer backbone via a carboxylic linker.

In another aspect of this embodiment, a second siRNA is conjugated to a nitrophenyl-carbonate. The nitrophenyl-carbonate reacts with the free amine to form a stable urethane linkage.

In another aspect of this embodiment, a second siRNA is conjugated to an isocyanate. The isocyanate reacts with the free amine to form a stable urea linkage.

In another aspect of this embodiment, a second siRNA is conjugated to an aldehyde. The aldehyde reacts with the free amine to form a reversible imine bond, which is reduced in situ to a stable secondary amine linkage by a suitable reducing agent such as sodium cyanoborohydride.

In another aspect, the second siRNA may be conjugated to PEG-succinimidyl succinate to form a linkage that is prone to hydrolytic cleavage in the endosome. Alternatively, the second siRNA may be conjugated to PEG-succinimidyl glutarate to form a linkage that is resistant to such cleavage. Examples of amine pegylation are shown in FIG. 2.

In another embodiment, a first siRNA is a thiol-modified siRNA, capable of reacting with a functional group on one or more ends of a linear or branched polymer. In one aspect of the embodiment, a second siRNA is conjugated to a maleimide. The maleimide reacts with the sulfhydryl group (—SH) of the thiol modified siRNA to form a stable thioether bond at pH 6.5-7.5.

In another aspect of this embodiment, a second siRNA is conjugated to a vinylsulfone. The vinylsulfone reacts with the sulfhydryl to form a stable thioether bond.

In another aspect of this embodiment, a second siRNA is conjugated to an orthopyridyl disulfide. The orthopyridyl disulfide reacts with the sulfhydryl to form a disulfide bond which is a reducible bond optionally subject to disruption within the endosome.

In another aspect of this embodiment, a second siRNA is conjugated to an acrylate group. The arylate reacts with the sulfhydryl to form an acid-labile B-thiopropionate linkage.

In another aspect of this embodiment, a second siRNA is conjugated to an iodoacetimide group. The iodoacetimide group reacts with the sulfhydryl to form a stable thioether bond.

In another embodiment, a first siRNA is linked by homobifunctional PEG to a second siRNA via a stable thioether linkage.

In another embodiment, a first siRNA is linked by homobifunctional PEG to a second siRNA via an acid-labile B-thiopropionate linkage.

In another embodiment, a first siRNA is linked by homobifunctional PEG to a second siRNA via an amide linkage.

In another embodiment, a first siRNA is linked by heterobifunctional PEG to a second siRNA via an amide and thioether linkage.

In another embodiment, four different siRNAs are linked by homomultifunctional PEG through a reversible B-thiopropionate linkage.

In another embodiment, six different siRNAs are linked by homomultifunctional PEG through an amide linkage.

In another embodiment, three different siRNAs are linked by heterobifunctional PEG.

In another embodiment, a first siRNA is linked to a second siRNA by a poly(beta-amino ester) polymer.

In one aspect of this embodiment, a first siRNA is linked to a second siRNA by a poly-(beta-amino ester) polymer in a fixed formulation.

In another embodiment, a first siRNA is linked to a second siRNA by a PEG-PLGA-PEG triblock polymer.

In another embodiment, a first siRNA is linked to a second siRNA by a PEG-PLGA-PEG triblock polymer.

In another embodiment, a first peptide-labeled siRNA is linked to a second peptide-labeled siRNA by PEG.

### BRIEF DESCRIPTION OF THE DRAWINGS

FIG. 1 depicts four methods of delivering siRNA agents: 1) co-encapsulation, where the agents are not linked, but are in the same vehicle; 2) separate encapsulation, where the agents are in separate vehicles; 3) molecular linkage, where the agents are covalently linked and delivered without a vehicle; or 4) covalently linked and encapsulated in a vehicle. Separate encapsulation has the advantage of easily changing the ratio of agents.

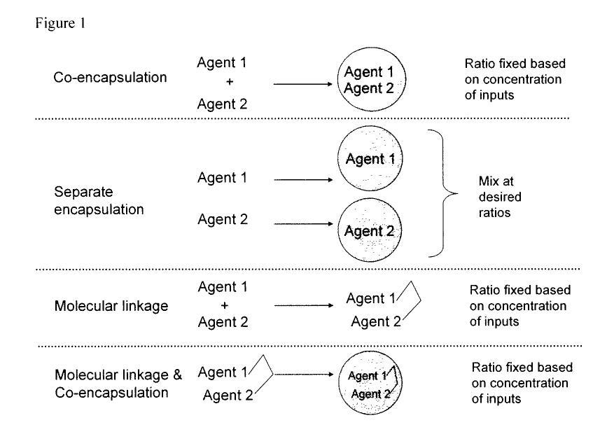

FIG. 2 depicts examples of chemical reactions involving a terminal amine, such as an amine modified siRNA. A polymeric linking agent with an attached amine-reactive moiety can be used to form a covalent bond with an amine functionalized siRNA. The reaction produces an amide bond form from an NHS ester (A and B), a urethane linkage from a NPC(C), a urea linkage from an isocyanate (D), and a secondary amine via condensation of the amine with an aldehyde moiety and subsequent reduction of the imine with sodium cyanoborohydride (E).

FIG. 3 depicts examples of chemical reactions involving a terminal thiol, such as a thiol modified siRNA. A polymeric linking agent with an attached thiol-reactive moiety can be used to form a covalent bond with a thiol functionalized siRNA. Thioethers are formed via Michael reactions with maleimide (A), vinyl sulfone (B), or acrylate (E). Alternatively, a disulfide can be formed via disulfide-thiolate exchange with an orthopyridyl disulfide moiety (C), or a thioether can be formed via an sn2 reaction with iodoacetamide (D).

FIG. 4 depicts asymmetric and symmetric formation of a bis-siRNA unit. When thiol modified siRNAs #1 and #2 (having different sequences) are reacted with the bismaleimide linking unit, the product mixture is a statistical 1:1 mixture of symmetric and asymmetric conjugate addition products.

FIG. 5 depicts acid labile thioether linkages made with bis-acrylate PEG moieties. The product mixture is a statistical 1:1 mixture of symmetric and asymmetric conjugate addition products.

FIG. 6 depicts the reaction scheme shows the methodology for making a specific tri-siRNA compound. Starting with linking units having an amine reactive moiety on one end and a thiol reactive moiety on the other, one can control the formation of the final product through the order of the addition of thiol modified, amine modified and thiol and amine modified reactants.

FIG. 7 depicts siRNAs expressing amine functionalities can also be co-polymerized with 1,4-butanediol diacrylate as shown in the reaction scheme. The length of the resulting polymer (molecular weight of the resulting polymeric molecules) is proportional to the length of reaction time.

FIG. 8 depicts a method of obtaining a block polymer linking unit. In this case, PLGA is given an amine functionality as shown in the reaction scheme, then the linking unit is completed by reacting the PLGA-diamine with two amine reactive PEG-siRNA compounds.

FIG. 9 depicts an example of one method of purifying mixtures of linked siRNAs using protein. The first step of the two-step process involves the use of nickel-nitrilotriacetic acid (Ni-NTA) resin to extract all HHHHHH-labeled species. Next, purification using anti-FLAG affinity gel extracts all species tagged with DYKDDDK.

FIG. 10 depicts co-polymers of two siRNA strands expressing a specific ratio of one siRNA strand to other can be achieved by the co-polymerization of amine functionalized siRNA strands using 1,4-butanediol diacrylate. Each siRNA strand is also functionalized with a dye (siRNA#1 with Cy3 and siRNA#2 with a Cy5-IEGRHHHHHH peptide conjugate). After polymerization, the resulting products are immobilized on Ni-NTA beads and sorted according to dye ratio using flow cytometry. The polymers are then cleaved from the beads using Factor Xa protease.

### BRIEF DESCRIPTION OF THE SEQUENCE LISTING

TABLE 1

| SEQ ID NO: # | Target |
|---|---|
| 1 | Influenza nucleocapsid protein sense |
| 2 | Influenza nucleocapsid protein antisense |
| 3 | EGFR sense |
| 4 | EGFR antisense |
| 5 | P13K sense |
| 6 | P13K antisense |
| 7 | Survivin sense |
| 8 | Survivin antisense |
| 9 | c-Myc sense |
| 10 | c-Myc antisense |
| 11 | Met sense |
| 12 | Met antisense |
| 13 | PDGFRA sense |
| 14 | PDGFRA antisense |
| 15 | PDGFRB sense |
| 16 | PDGFRB antisense |

### DETAILED DESCRIPTION OF THE INVENTION

### Definitions

As used herein, the term “PEG” represents certain polyethylene glycol containing substituents having a designated number of ethylene glycol subunits. The term “PEG(z)” represents polyethylene glycol containing “z” ethylene glycol subunits. The term PEG also includes PEG-polymer of polyethylene glycol units; the polymer being linear, multiarmed or branched.

The term “polyethylene glycol-based linker” or “PEG-based linker” refers to a linking agent having a structure according to formula II:

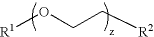

in which
z is an integer from 1 to 10,000, preferably from 1 to 500, by way of example from 1 to 100 or 1 to 50 or 1 to 20 and more preferably from 1 to 10.
R¹ and R² are divalent organic radicals independently selected from substituted or unsubstituted alkyl, substituted or unsubstituted heteroalkyl and substituted or unsubstituted aryl.

Preferably R¹ and R² are independently selected from —C(O)R³, —SR³, —NHR³ and —OR³, in which R³ is H, substituted or unsubstituted alkyl, substituted or unsubstituted heteroalkyl, acyl, —OR⁴, —C(O)R⁴, —C(O)OR⁴, —C(O)NR⁴R⁵, —P(O)(OR⁴)₂, —C(O)CHR⁴R⁵, —NR⁴R⁵, —N(+)R⁴R⁵R⁶, —SR⁴ or SiR⁴R⁵R⁶. The symbols R⁴, R⁵ and R⁶ independently represent H, substituted or unsubstituted alkyl, substituted or unsubstituted heteroalkyl and substituted or unsubstituted aryl, wherein R⁴ and R⁵ together with the nitrogen atom to which they are attached are optionally joined to form a substituted or unsubstituted heterocycloalkyl ring system having from 4 to 6 members, optionally containing two or more heteroatoms.

Advantageously, the PEG-based linker according to the invention is flexible, non-immunogenic, not susceptible to cleavage by proteolytic enzymes and enhances the solubility in aqueous media of the nucleic acid conjugates.

In another embodiment, the PEG enhances the solubility in aqueous media of the PEG nucleic acid conjugates.

The term “alkyl” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3 (1,4-pentadienyl), ethynyl, 1- and 3-propynyl, 3-butynyl, and the higher homologs and isomers. The term “alkyl” unless otherwise noted, is also meant to include those derivatives of alkyl defined in more detail below, such as “heteroalkyl.” Alkyl groups, which are limited to hydrocarbon groups, are termed “homoalkyl.”

The term “alkylene” by itself or as part of another substituent means a divalent radical derived from an alkane, as exemplified, but not limited, by —CH2-CH2-CH2-CH2-, and further includes those groups described below as “heteroalkylene”. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen, carbon and sulfur atoms may optionally be oxidized and the nitrogen heteroatom may optionally be quaternized. The heteroatom (s) O, N and S and Si may be placed at any interior position of the heteroalkyl group, at the end of the heteroalkyl group or at the position at which the alkyl group is attached to the remainder of the molecule. Examples include, but are not limited to, —CH₂, CH₂—OCH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃ and CH═CH—N(CH₃)—CH₃. Up to two heteroatoms may be consecutive, such as, for example, —CH₂—NH—OCH₃ and —CH₂—O—S—(CH₃)₃. Similarly, the term “heteroalkylene” by itself or as part of another substituent means a divalent radical derived from heteroalkyl, as exemplified, but not limited by, —CH₂—CH₂—SCH₂—CH₂-and-CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives. Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “lower” in combination with the terms “alkyl” or “heteroalkyl” refers to a moiety having from 1 to 6 carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C¹—C⁴) alkyl” is meant to include, but not be limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, arylalkyl) includes both aryl and heteroaryl rings as defined above.

Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy) propyl, and the like).

Each of the above terms (e.g., “alkyl,” “heteroalkyl”, “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents”, respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″ C(O)₂R′, —NR—C(NR′R″)═NR″″, —NR—C(NR′R″)═NR′″, —S(O)R′, —S(O)₂R′, —S(O)₂NR′R″, —NRSO₂R′, NRR′SO₂R″, —CN and —NO₂ in a number ranging from zero to (2m′+1), where m′ is the total number of carbon atoms in such radical. R′, R″, R′″ and R“ ” each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R“ ” groups when more than one of these groups is present. When R′ and R″ are attached to the same nitrogen atom, they can be combined with the nitrogen atom to form a 5-, 6-, or 7-membered ring. For example, NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, C(O)CH₂OCH₃, and the like).

Similar to the substituents described for the alkyl radical, the aryl substituents and heteroaryl substituents are generally referred to as “aryl substituents” and “heteroaryl substituents”, respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen(C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)— (CRR′)q-U—, wherein T and U are independently NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH2)r-B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)s-X—(CR″R′″)d-, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆) alkyl.

As used herein, the term “heteroatom” includes oxygen (O), nitrogen (N), sulfur (S) and silicon (Si).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocyclyl groups.

In one embodiment, the PEG-based linker has the formula III:

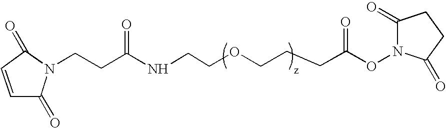

wherein z (representing the number of ethylene glycol subunits) is an integer from 1 to 10.000, preferably from 1 to 500, more preferably from 1 to 100.
By way of example, if z=4, then the PEG-based linker has the formula IV:

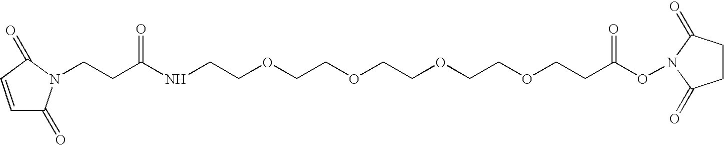

The formula V below shows the PEG-based linker of formula III once linked to a nucleic acid molecule (e.g. a siRNA):

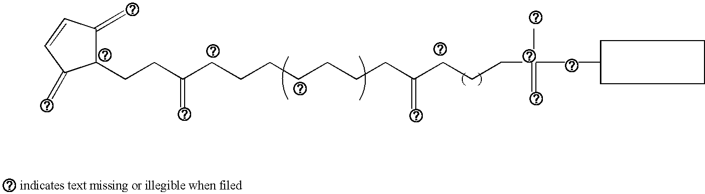

wherein:
z is an integer from 1 to 10.000, preferably from 1 to 500, more preferably from 1 to 100;
k is an integer from 1 to 250, preferably from 1 to 100 and more preferably from 1 to 10.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The term “nucleotide analog,” also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retard the ability of the nucleotide analog to perform its intended function. The terms “nucleotide” and “nucleotide analog” can be used interchangeably.

The term “oligonucleotide” (“ON”) refers to a short polymer of nucleotides and/or nucleotide analogs.

The term “nucleic acid analog(s)” refers to structurally modified, polymeric analogs of DNA and RNA made by chemical synthesis from monomeric nucleotide analog units, and possessing some of the qualities and properties associated with nucleic acids.

The term “RNA” or “RNA molecule” or “ribonucleic acid molecule” refers to a polymer of ribonucleotides. The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be posttranscriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that encodes the amino acid sequence of one or more polypeptide chains. This information is translated during protein synthesis when ribosomes bind to the mRNA.

The terms “polynucleotide(s),” “nucleic acid(s)” or “nucleic acid molecule(s)” are used interchangeably herein and refer to a polymer of nucleotides joined together by a phosphodiester linkage between 5′ and 3′ carbon atoms. Polynucleotide(s), nucleic acid(s) or nucleic acid molecule(s) and their analogs can be linear, circular, or have higher orders of topology (e.g., supercoiled plasmid DNA). DNA can be in the form of antisense, plasmid DNA, parts of a plasmid DNA, vectors (e.g., P1-derived Artificial Chromosome, Bacterial Artificial Chromosome, Yeast Artificial Chromosome, or any artificial chromosome), expression cassettes, chimeric sequences, chromosomal DNA, or derivatives of these groups. RNA can be in the form of oligonucleotide RNA, tRNA (transfer RNA), snRNA (small nuclear RNA), rRNA (ribosomal RNA), mRNA (messenger RNA), antisense RNA, (interfering) double-stranded and single-stranded RNA, ribozymes, chimeric sequences, or derivatives of these groups. Nucleic acids can be single (“ssDNA”), double (“dsDNA”), triple (“tsDNA”), or quadruple (“qsDNA”) stranded DNA, and single stranded RNA (“RNA”) or double stranded RNA (“dsRNA”).

“Multistranded” nucleic acid contains two or more strands and can be either homogeneous as in double stranded DNA, or heterogeneous, as in DNA/RNA hybrids. Multistranded nucleic acid can be full length multistranded, or partially multistranded. It can further contain several regions with different numbers of nucleic acid strands. Partially single stranded DNA is considered a sub-group of ssDNA and contains one or more single stranded regions as well as one or more multiple stranded regions.

The term “oligoribonucleotide” refers to a short polymer of ribonucleotides and/or chemically modified ribonucleotides. The term “ribonucleotide” refers to a nucleotide that contains the sugar ribose. The ribonucleotide may occur as a constituent of dsRNA, siRNA, miRNA or shRNA. Ribonucleotides are composed of naturally-occurring ribonucleobases, sugars and covalent internucleoside linkages. The terms “modified ribonucleotide,” “ribonucleotide analog” and “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. The oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Exemplary RNA analogues include sugar- and/or backbone-modified ribonucleotides and/or deoxyribonucleotides.

Such alterations or modifications can further include addition of non-nucleotide material, such as to the end(s) of the RNA or internally (at one or more nucleotides of the RNA). RNA analog needs only to be sufficiently similar to natural RNA that it has the ability to mediate (mediates) RNA interference.

The terms “target sequence” and “target nucleic acid” refer to a sequence of a gene product of interest that may be downregulated, modulated or silenced through RNAi. The foregoing terms may encompass any nucleic acid capable of being targeted, including, without limitation, DNA, RNA (including pre-mRNA and mRNA and portions thereof) transcribed from such DNA and also cDNA derived from such RNA. In some embodiments of this invention, modulation of gene expression is achieved by modulation of RNA associated with a particular gene RNA. More particularly, the invention provides a means of modulating target nucleic acids where the target nucleic acid is messenger RNA. The mRNA is degraded via RNAi as well as by other mechanisms where double stranded RNA are recognized and degraded, cleaved or otherwise rendered inoperable.

The terms “RNA interference,” “interfering RNA” or “RNAi” refer to double-stranded RNA (i.e., duplex RNA) that is capable of reducing or inhibiting expression of a target gene by mediating the degradation of mRNAs which are complementary to the sequence of the interfering RNA when the interfering RNA is in the same cell as the target gene. Interfering RNA refers to a double-stranded RNA formed by two complementary strands or by a single self-complementary strand. Interfering RNA may have substantial or complete identity to the target gene or may comprise a region of mismatch. Interfering RNA includes “small interfering RNA or “siRNA,” which are RNA duplexes of about 15-60, 15-50 or 15-40 (duplex) nucleotides in length, more typically about 15-30, 15-25 or 19-25 (duplex) nucleotides in length, and are preferably about 20-24, 21-22, or 21-23 (duplex) nucleotides in length. Each complementary sequence of the double-stranded siRNA may be 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded oligoribonucleotide molecule assembled from two separate stranded molecules, wherein one strand is the sense strand and the other is the complementary anti-sense strand; a double-stranded oligoribonucleotide molecule assembled from a single stranded molecule, where the sense and anti-sense regions are linked by a nucleic acid-based or non-nucleic acid-based linker; a double-stranded oligoribonucleotide molecule with a hairpin secondary structure having self-complementary sense and anti-sense regions.

The term “RNA Co-Interference” (“RNAco-I”) refers to the use of two or more RNAi conferring agents, comprising two or more double-stranded oligoribonucleotides, such as siRNA, to one or more distinct targets.

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al, Proc. Natl. Acad. Sci. USA, 99:14236 (2002); Byrom et al., Ambion Tech Notes, 10(1):4-6 (2003); Kawasaki et al, Nucleic Acids Res., 31:981-987 (2003); Knight et al, Science, 293:2269-2271 (2001); and Robertson et al, J Biol Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

As used herein, the term “region of mismatch” or “mismatch region” refers to a portion of a siRNA sequence that does not have 100% complementarity to its target sequence. A siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five or more nucleotides.

The terms “substantially identical” or “substantial identity,” when comparing two or more nucleic acids, refers to two or more sequences or subsequences that are the same or have a specified percentage of nucleotides that are the same (i.e., at least about 60%, preferably at least about 65%, 70%, 75%, 80%, 85%, 90%, or 95% identity over a specified region), when compared and aligned to yield maximum correspondence over a specified region. The comparison may be performed using one of the following sequence comparison algorithms or by manual alignment and visual inspection. Comparative sequence alignments can be conducted, e.g., by the local homology algorithm of Smith and Waterman, Adv. Appl. Math., 2:482 (1981), by the homology alignment algorithm of Needleman and Wunsch, J. Mol. Biol, 48:443 (1970), by the search for similarity method of Pearson and Lipman, Proc. Natl. Acad. Sd. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. (1995 supplement)).

In comparing sequences, typically one sequence acts as a reference sequence, to which test sequences are compared. When using a sequence comparison algorithm as specified above, test and reference sequences are entered into a computer, subsequence coordinates are designated, if necessary and sequence algorithm program parameters are designated. Default program parameters can be used, or alternative parameters can be designated. The sequence comparison algorithm then calculates the percent sequence identities for the test sequences relative to the reference sequence, based on the program parameters.

The specified region to be compared includes reference to a segment of any one of a number of contiguous positions selected from the group consisting of from about 5 to about 60, usually about 10 to about 45, more usually about 15 to about 30, in which a sequence may be compared to a reference sequence of the same number of contiguous positions after the two sequences are optimally aligned. Methods of alignment of sequences for comparison are well known in the art.

This definition, when the context indicates, also refers analogously to the complement of a sequence. Preferably, the substantial identity exists over a region that is at least about 5, 10, 15, 20, 25, 30, 35, 40, 45, 50, 55 or 60 nucleotides in length.

As used herein, the term “short interfering RNA” (“siRNA”) (also referred to in the art as “small interfering RNAs”) refers to a RNA (or RNA analog) comprising between about 10-50 nucleotides (or nucleotide analogs) which is capable of directing or mediating RNA interference.

The terms “complementary” and “complementarity” refer to the capability of two nucleobases to precisely pair regardless of where the two are located. For instance, a nucleobase located at a certain position of an oligoribonucleotide, which is capable of hydrogen bonding with a nucleobase at a certain position of a target nucleic acid, is considered to be in a complementary position with respect to the hydrogen bonding between the oligoribonucleotide and the target nucleic acid. Thus, the oligoribonucleotide and the target nucleic acid are considered to be complementary to each other when a sufficient number of complementary positions in each molecule are occupied by nucleobases that can hydrogen bond with each other. The terms “specifically hybridizable” and “complementary” are terms which are used to indicate a sufficient degree of precise pairing or complementarity over a sufficient number of nucleobases such that stable and specific binding occurs between the oligoribonucleotide and a target nucleic acid. Complementarity is achieved through hybridization.

In the context of this invention, “hybridization” or “hybridizing” denotes pairing of complementary strands of oligomeric compounds. Pairing typically involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases that pair through the formation of hydrogen bonds. Hybridization can occur under varying circumstances.

In the present invention the phrase “stringent hybridization conditions” or “stringent conditions” refers to conditions under which an oligomeric compound of the invention will hybridize to its target sequence, but to a minimal number of other sequences. Stringent conditions are sequence-dependent and will vary with different circumstances and in the context of this invention; “stringent conditions” under which oligomeric compounds hybridize to a target sequence are determined by the nature and composition of the oligomeric compounds and the assays in which they are being investigated.

Accordingly, it is understood in the art that the sequence of complementary oligomeric and target nucleic acid compounds need not be 100% to be considered as specifically hybridizable. Oligoribonucleotides may hybridize over one or more segments such that intervening or adjacent segments are not involved in the hybridization. This may occur with so-called loop or hairpin structures. In some embodiments of the invention, oligomeric compounds of the present invention comprise at least 70% sequence complementarity to a target region within the target nucleic acid, in further embodiments they comprise 90% sequence complementarity and in yet further embodiments they comprise 95% sequence complementarity to the target region within the target nucleic acid sequence to which they are targeted. For example, an oligomeric compound in which 18 of 20 nucleobases of the oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

A siRNA having a “sequence sufficiently complementary to a target mRNA sequence to direct target-specific RNA interference (RNAi)” means that the siRNA has a sequence sufficient to trigger the destruction of the target mRNA by the RNAi machinery or process, i.e. there is preferably greater than 80% sequence identity, or more preferably greater than 90% 91%, 92%, 93%, 94%, 95%, 96%, 97%, 98%, 99%, or even 100% sequence identity, between the siRNA and the portion of the target mRNA sequence encoded by the target gene.

The term “cleavage site” refers to the residues, e.g., nucleotides, at which RISC cleaves the target RNA, e.g., near the center of the complementary portion of the target RNA, e.g., about 8-12 nucleotides from the 5′ end of the complementary portion of the target RNA.

The term “upstream of the cleavage site” refers to residues, e.g., nucleotides or nucleotide analogs, 5′ to the cleavage site. Upstream of the cleavage site with reference to the antisense strand refers to residues, e.g., nucleotides or nucleotide analogs 5′ to the cleavage site in the antisense strand.

The term “downstream of the cleavage site” refers to residues, e.g., nucleotides or nucleotide analogs, located 3′ to the cleavage site. Downstream of the cleavage site with reference to the antisense strand refers to residues, e.g., nucleotides or nucleotide analogs, 3′ to the cleavage site in the antisense strand.

The term “phosphorylated” means that at least one phosphate group is attached to a chemical (e.g., organic) compound. Phosphate groups can be attached, for example, to proteins or to sugar moieties via the following reaction: free hydroxyl group+phosphate donor gives phosphate ester linkage. The term “5′ phosphorylated” is used to describe, for example, polynucleotides or oligonucleotides having a phosphate group attached via ester linkage to the C5 hydroxyl of the 5′ sugar (e.g., the 5′ ribose or deoxyribose, or an analog of same). Mono-, di-, and tri-phosphates are common. Also intended to be included within the scope of the invention are phosphate group analogs which function in the same or similar manner as the mono-, di-, or triphosphate groups found in nature (see e.g., exemplified analogs).

The term “RNA co-interference composition” refers to a short polymer of ribonucleotides and/or chemically modified ribonucleotides containing one or more chemical linkages joining two or more double-stranded oligoribonucleotides. The term “ribonucleotides” refers to ribonucleotides composed of naturally-occurring ribonucleobases, sugars and covalent internucleoside linkages. The terms “modified ribonucleotide” and “ribonucleotide analog” refer to ribonucleotides that have one or more non-naturally occurring moieties, which function in a similar manner to naturally occurring ribonucleotides. Such modified ribonucleotides may be advantageous with respect to conferring enhanced cellular uptake of the molecule, enhanced affinity for a specified target sequence and increased stability of the molecule in the presence of nucleases.

The term “contiguous” is used to denote and define a group of ribonucleotides forming a region. The first region would constitute a ds siRNA molecule capable of initiating RNAi. Ribonucleotides which are adjacent to each other in the polyribonucleotide backbone and also bound between strands through Watson-Crick hybridization are contemplated by the term.

A “target sequence” or “target nucleic acid” refers to a sequence of a gene product of interest that may be downregulated, modulated or silenced through RNAi. The foregoing terms may encompass any nucleic acid capable of being targeted, including, without limitation, DNA, RNA (including pre-mRNA and mRNA and portions thereof) transcribed from such DNA, and also cDNA derived from such RNA. In some embodiments of this invention, modulation of gene expression is achieved by modulation of RNA associated with a particular gene RNA. More particularly, the invention provides a means of modulating target nucleic acids where the target nucleic acid is messenger RNA. The mRNA is degraded via RNAi as well as by other mechanisms where double stranded RNA are recognized and degraded, cleaved or otherwise rendered inoperable.

The present invention provides for both prophylactic and therapeutic methods of treating a subject at risk of (or susceptible to) a disease, disorder or condition or trait associated with aberrant or unwanted target gene expression or activity. “Treatment”, or “treating” as used herein, is the application or administration of a therapeutic agent comprising one or more double-stranded oligoribonucleotides to a subject, or application or administration of a therapeutic agent to an isolated tissue or cell line from a subject, who has a disease or disorder, condition or trait, a symptom of such a disease, disorder, condition or trait, or a predisposition toward a disease, disorder, condition or trait, with the purpose to cure, heal, alleviate, relieve, alter, remedy, ameliorate, improve or affect the disease, disorder, condition or trait, or the symptoms of, or predisposition toward, said disease, disorder, condition or trait. A subject includes without limitation a mammal and specifically a human being. The double-stranded oligoribonucleotide may be administered in an amount sufficient for degradation of one or more target nucleic acids to occur, thereby treating the disease, disorder, condition or trait associated with the protein. Prophylactic and therapeutic methods of treatment may be specifically tailored or modified, based on knowledge obtained from the field of pharmacogenomics.

“Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

As used herein a “biologically active molecule/agent/moiety” is any molecule/agent that has some biological effect. Biologically active agents include proteins, chemical compounds, nucleic acids, polysaccharides (which include monosaccharides, disaccharides and oligosaccharides), small molecules and peptide therapeutics. Nucleic acids include DNA, RNA, antisense oligonucleotides, antisense miRNA inhibitors (anti-miRs, antagomiRs), interfering RNA (RNAi), small interfering RNA (siRNA), shRNAs, miRNAs, ribozymes, DNAzymes, triplex forming oligonucleotides, etc. The nucleic acids can be single-, double-, triple-stranded, etc. Biologically active agents also include the other therapeutic agents/drugs described herein.

A “loss-of-function” phenotype results from the knock-out or knock-down of a target gene or target genes. A selected gene can be knocked down by use of a double stranded oligoribonucleotide of the invention and the resultant phenotype can be observed. However, knockdown of an essential gene could have a lethal or toxic effect and may also have off-target consequences. In some cases it is desirable to provide to the cell a double stranded oligoribonucleotide that inhibits expression of the protein via the target gene without reaching a level of maximum efficiency. Such oligoribonucleotides may be constructed and/or formulated to yield reduced RNAi thereby inhibiting expression of the protein translated from the targeted gene sequence less than maximally. Suitable concentrations of a double stranded oligoribonucleotide used for this purpose include concentrations that do not maximally inhibit RNAi activity and which ameliorate the undesirable effect of the double-stranded oligoribonucleotide. Reduced knock-down activity can be determined using dual fluorescence assays described in the art, for example in Example 1 of US Patent Publication No. 2005/0020521 which is incorporated by reference herein. In some cases, a useful double stranded oligoribonucleotide of the invention may be one that inhibits RNAi by less than 100%. For example, useful double stranded oligoribonucleotide of the invention derivative that is useful for reducing the RNAi effect may be a double stranded oligoribonucleotide that can inhibit RNAi activity by less than 100%, e.g., 90%, 75%, 50%, 25%, or 10%.

### RNAco-i

The instant invention teaches compositions for, method to produce, and uses of RNAco-i. RNAco-i is a novel approach to therapeutic development and administration based on the notion that intelligent combinations of drugs when co-administered in a fundamentally bio-active manner, can have profound improvements in therapy. The combinations involved in RNAco-i consist of two or more bioactive agents. In a preferred embodiment, the first agent is an siRNA, which is bound to a second siRNA. In this embodiment, the two siRNAs are preferentially targeted at different genes. Alternatively, they can target different genetic sequences of a common gene. In another embodiment, the second agent can be more than one siRNA, with the synthesis and linkage process used in series (or parallel) to add multiple siRNAs together into a single entity. In another embodiment an additional agent can be in whole or in part, a non-RNAi-conferring agent. Such non-RNAi-conferring agents include, but are not limited to small molecules, peptides, proteins, polysaccharides, lipids, and other nucleic acids. Linking the siRNAs allows a composition wherein the relative concentrations of the multiplicity of bioactive agents is specifically known, particularly through the methods taught herein. Other means of conferring RNAi, such as shRNA or miRNA can also be readily employed, as can other substances that can reduce if not eliminate the activity of a gene product. The instant invention specifically teaches means to define the various active agents, which in preferred embodiments have synergistic effects. This invention also teaches how to produce a functional means of producing a delivery vehicle, and the use to treat important diseases among other applications.

### Synthesis of RNAco-i Vehicles for Co-Delivery

Essential to the administration of functional RNAco-i, is an effective formulation. Previous methods have described numerous approaches of conjugating oligonucleotides such as siRNA and other active agents to formulation agents and other such moieties. For example, a disulfide linkage has also been utilized at the 3′ terminus of an oligonucleotide to link a peptide to the oligonucleotide as is described by Corey, et al., Science 1987, 238, 1401; Zuckermann, et al., J. Am. Chem. Soc. 1988, 110, 1614; and Corey, et al., J. Am. Chem. Soc. 1989, 111, 8524. Similarly, Drezek and colleagues disclose methods of linking a sense and an antisense strand via a polymeric loop, such as a heterobifunctional PEG (WO 07/11806 “siRNA nanoprobes”). However, in this invention only a single, optionally functionally active, siRNA molecule is delivered with the individual strands separated via a polymeric linker. Additionally, Kim and colleagues similarly disclose methods of linking a singly synthetic siRNA to one end of a polymer (WO 2007/021142 “Sirna-hydrophilic polymer conjugates for intracellular delivery of siRNA and method thereof). The instant invention uniquely teaches methods of combining two or more active agents, including at least one siRNA into a common formulation.

In yet another aspect, the present invention provides a nucleic acid-lipid particle comprising a modified siRNA described herein, a cationic lipid, and a non-cationic lipid. In certain instances, the nucleic acid-lipid particle further comprises a conjugated lipid that inhibits aggregation of particles. Preferably, the nucleic acid-lipid particle comprises a modified siRNA described herein, a cationic lipid, a non-cationic lipid and a conjugated lipid that inhibits aggregation of particles.

The cationic lipid may be, e.g., N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxypropylamine (DODMA), 1,2-DiLinoleyloxy-N,N-dimethylaminopropane (DLinDMA), 1,2-Dilinolenyloxy-N,N-dimethylaminopropane (DLendMA), or mixtures thereof. The cationic lipid may comprise from about 20 mol % to about 50 mol % or about 40 mol % of the total lipid present in the particle.

The non-cationic lipid may be an anionic lipid or a neutral lipid including, but not limited to, distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylcholine (DPPC), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), cholesterol, or mixtures thereof. The non-cationic lipid may comprise from about 5 mol % to about 90 mol % or about 20 mol % of the total lipid present in the particle.

The conjugated lipid that inhibits aggregation of particles may be a polyethyleneglycol (PEG)-lipid conjugate, a polyamide (ATTA)-lipid conjugate, a cationic-polymer-lipid conjugates (CPLs), or mixtures thereof. In one preferred embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be a PEG-dilauryloxypropyl (C12), a PEG-dimyristyloxypropyl (C14), a PEG-dipalmityloxypropyl (C16), or a PEG-distearyloxypropyl (C18). In some embodiments, the conjugated lipid that inhibits aggregation of particles is a CPL that has the formula: A-W—Y, wherein A is a lipid moiety, W is a hydrophilic polymer, and Y is a polycationic moiety. W may be a polymer selected from the group consisting of PEG, polyamide, polylactic acid, polyglycolic acid, polylactic acid/polyglycolic acid copolymers, or combinations thereof, the polymer having a molecular weight of from about 250 to about 7000 daltons. In some embodiments, Y has at least 4 positive charges at a selected pH. In some embodiments, Y may be lysine, arginine, asparagine, glutamine, derivatives thereof or combinations thereof. The conjugated lipid that prevents aggregation of particles may be from 0 mol % to about 20 mol % or about 2 mol % of the total lipid present in the particle.

In some embodiments, the nucleic acid-lipid particle further comprises cholesterol at, e.g., about 10 mol % to about 60 mol %, about 30 mol % to about 50 mol %, or about 48 mol % of the total lipid present in the particle.

In certain embodiments, the modified siRNA in the nucleic acid-lipid particle is not substantially degraded after exposure of the particle to a nuclease at 37° C. for at least 20, 30, 45, or 60 minutes; or after incubation of the particle in serum at 37° C. for at least 30, 45, or 60 minutes.

In some embodiments, the modified siRNA is fully encapsulated in the nucleic acid-lipid particle. In other embodiments, the modified siRNA is complexed with the lipid portion of the particle.

The present invention further provides pharmaceutical compositions comprising the nucleic acid-lipid particles described herein and a pharmaceutically acceptable carrier.

In still yet another aspect, the modified siRNA described herein is used in methods for silencing expression of a target sequence. In particular, it is an object of the present invention to provide in vitro and in vivo methods for treatment of a disease or disorder in a mammal by downregulating or silencing the transcription and/or translation of a target gene of interest. In one embodiment, the present invention provides a method for introducing an siRNA that silences expression (e.g., mRNA and/or protein levels) of a target sequence into a cell by contacting the cell with a modified siRNA described herein. In another embodiment, the present invention provides a method for in vivo delivery of an siRNA that silences expression of a target sequence by administering to a mammal a modified siRNA described herein. Administration of the modified siRNA can be by any route known in the art, such as, e.g., oral, intranasal, intravenous, intraperitoneal, intramuscular, intra-articular, intralesional, intratracheal, subcutaneous, or intradermal.

In these methods, the modified siRNA is typically formulated with a carrier system, and the carrier system comprising the modified siRNA is administered to a mammal requiring such treatment. Examples of carrier systems suitable for use in the present invention include, but are not limited to, nucleic acid-lipid particles, liposomes, micelles, virosomes, nucleic acid complexes {e.g., lipoplexes, polyplexes, etc.), and mixtures thereof. The carrier system may comprise at least one, two, three, four, five, six, seven, eight, nine, ten or more of the modified siRNA molecules described herein. Alternatively, cells are removed from a mammal such as a human, the modified siRNA is delivered in vitro and the cells are then administered to the mammal, such as by injection.

In some embodiments, the modified siRNA is in a nucleic acid-lipid particle comprising the modified siRNA, a cationic lipid and a non-cationic lipid. Preferably, the modified siRNA is in a nucleic acid-lipid particle comprising the modified siRNA, a cationic lipid, a non-cationic lipid, and a conjugated lipid that inhibits aggregation of particles. A therapeutically effective amount of the nucleic acid-lipid particle can be administered to the mammalian subject (e.g., a rodent such as a mouse or a primate such as a human, chimpanzee or monkey).

In some embodiments, the modified nucleotide includes, but is not limited to, 2′OMe nucleotides, 2° F. nucleotides, 2′-deoxy nucleotides, 2′OMOE nucleotides, LNA nucleotides and mixtures thereof. In preferred embodiments, the modified nucleotide comprises a 2′OMe nucleotide (e.g., 2′OMe purine and/or pyrimidine nucleotide) such as, for example, a 2′OMe-guanosine nucleotide, 2′OMe-uridine nucleotide, 2′OMe-adenosine nucleotide, 2′OMe-cytosine nucleotide and mixtures thereof. In certain instances, the modified nucleotide is not a 2′OMe-cytosine nucleotide.

In order to produce a means to effectively administer RNAco-i, the two agents are combined into a single entity. By combining the two agents into a common vehicle, the entity may be viewed as a single agent or as a prodrug, which can have significant impact in facilitating trial design. As such, while it is well known in the art that multiple active agents [see for example, U.S. Pat. Nos. 6,693,125 “Combinations of drugs (e.g. a benzimidazole and pentamidine) for the treatment of neoplastic disorders,” 6,569,853 “Combinations of chlorpromazine and pentamidine for the treatment of neoplastic disorders”, 7,253,155 “Combinations for the treatment of immunoinflammatory disorders”] can be delivered in a single formulation, such as a pill, injection, etc., the benefits describes herein relate only when the multiple active agents are conjugated or otherwise bound to the carrier. Essential to this, this entity must be formed such that the two agents retain function.

Modifications may be made to the sense and/or antisense strands. siRNAs are linked through the 5′ or the 3′ ends of the modified strands either at the terminal 3′ or 2′-OH. Alternatively, one or more internal nucleotides from the sense or antisense strand are modified to facilitate linkage between the siRNA species. The linkage site may be common between multiple siRNAs or distinct for each siRNA. In a preferred embodiment, the siRNAs are linked through the 5′ or 3′ ends of their sense strand, leaving their antisense strands unmodified (with respect to linker chemistry). Previous studies have demonstrated that a single 2-hydroethylphosphate substitution of the antisense strand abolished RNAi activity, whereas the same modification of the sense strand did not interfere with the siRNA functionality [Hamada M et al. “Effects on RNA interference in gene expression (RNAi) in cultured mammalian cells of mismatches and the introduction of chemical modifications at the 3′-ends of siRNAs.” Antisense Nucleic Acid Drug Dev (2002) 12(5):301-9].

The group of modified nucleotides and/or the group of flanking nucleotides may comprise a number of nucleotides whereby the number is selected from the group comprising one nucleotide to 10 nucleotides. In connection with any ranges specified herein, it is to be understood that each range discloses any individual integer between the respective figures used to define the range including said two figures defining said range. In the present case the group thus comprises one nucleotide, two nucleotides, three nucleotides, four nucleotides, five nucleotides, six nucleotides, seven nucleotides, eight nucleotides, nine nucleotides and ten nucleotides.

One synthetic 2′-modification that imparts increased nuclease resistance and a very high binding affinity to nucleotides is the 2-methoxyethoxy (2′-MOE, 2′-OCH₂, CH₂, OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligonucleotides having the 2′-MOE modification displayed improved RNA affinity and higher nuclease resistance. Chimeric oligonucleotides having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states.

The double stranded structure of the siRNA may be blunt ended, on one or both sides. More specifically, the double stranded structure may be blunt ended on the double stranded structure's side which is defined by the 5′-end of the first strand and the 3′-end of the second strand, or the double stranded structure may be blunt ended on the double stranded structure's side which is defined by the 3′-end of the first strand and the 5′-end of the second strand.

Additionally, at least one of the two strands may have an overhang of at least one nucleotide at the 5′-end; the overhang may consist of at least one deoxyribonucleotide. At least one of the strands may also optionally have an overhang of at least one nucleotide at the 3′-end.

Linkage of siRNA species can be achieved through a number of means. In certain embodiments, the siRNA species are directly linked, for example through a shared 5′ phosphate. In preferred embodiments, the siRNA species are linked to a distinct carrier moiety. These moieties are can include, but are not limited to, a nucleic acid, a lipid, a sugar, a protein, a peptide, or a polymer.

In some embodiments the linkage can be through a pre-defined non-bioactive strand of RNA or DNA. This linkage can include a homopolymer (e.g. CCCCCCC), a heteropolymer (e.g. ATATATAT or AUAUAUAU), or other sequences of RNA or DNA that do not have an independent biological function. Preferentially, such RNA or DNA linkages are of a length such that the siRNA or other active agent can be released by the activity of Dicer. These lengths can be 19, 20, 21, 22, 23, 24, 25, 26, 28, 29, 30 or more nucleic acids. In this aspect of the invention, the cleavage of the non-bioactive nucleic acid strand allows for the release of free siRNA or other RNAi conferring agents. As such, the RNAi activity occurs in a manner where it is clearly not confounded by the use of the co-delivery vehicle, such as through co-location with another drug, steric hinderance, multiplicity (and distinctiveness) of active sites, etc.

Oligoribonucleotides can be covalently joined through a linking moiety. In some embodiments, the linking moiety comprises a chain structure or an oligomer of repeating units such as nucleosides, ethylene glycol or amino acid units. The linker can have at least two functionalities for joining two or more oligoribonucleotides. Linking moieties can comprise functionalities that are electrophilic for reacting with nucleophilic groups on the oligoribonucleotide, or nucleophilic for reacting with electrophilic groups on an oligoribonucleotide. In some embodiments, linker functionalities include amino, hydroxyl, carboxylic acid, thiol, phosphoramidate, phosphate, phosphite, unsaturations (e.g., double or triple bonds), and the like. Some exemplary linking moities include: poly(ethylene glycol), poly(1,3-propylene glycol), poly(lactic acid), poly(glycolic acid), poly(lactic-co-glycolic acid), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid and poly(hydroxyalkanoate), wherein said linking moiety further comprises at least two terminal reactive groups corresponding and reactive with two or more functional groups selected from the group consisting of: OH, —COOH, N-hydroxy succidimidyl ester, Imidazole amide, triazole amide, tetrazole amide, hydroxy benzotriazole ester, 1-hydroxy-7-azabenzotriazole ester, 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate, isocyanate, optionally substituted aldehyde, optionally substituted ketone, optionally substituted acrylate, maleimide, vinyl sulfone, and orthopyridyl disulfide.

Many additional linking moieties are known in the art that can be useful in the attachment of oligoribonucleotides. A review of many of the useful linker groups can be found in, for example, Antisense Research and Applications, S. T. Crooke and B. Lebleu, Eds., CRC Press, Boca Raton, Fla., 1993, p. 303-350.

Linkers and their use in preparation of conjugates of oligomeric compounds are provided throughout the art such as in WO 96/11205 and WO 98/52614 and U.S. Pat. Nos. 4,948,882; 5,525,465; 5,541,313; 5,545,730; 5,552,538; 5,580,731; 5,486,603; 5,608,046; 4,587,044; 4,667,025; 5,254,469; 5,245,022; 5,112,963; 5,391,723; 5,510475; 5,512,667; 5,574,142; 5,684,142; 5,770,716; 6,096,875; 6,335,432; and 6,335,437, each of which is incorporated by reference in its entirety.

In some embodiments, linking moieties can be attached to the terminus of an oligoribonucleotide such as a 5′ or 3′ terminal residue of a nucleic acid. Linking moieties can also be attached to internal residues of the oligoribonucleotide. For double-stranded oligoribonucleotides, linking moieties can be attached to one or both strands. In some embodiments, a double-stranded oligoribonucleotide contains a linking moiety attached to the sense strand. In other embodiments, a double-stranded oligoribonucleotide contains a conjugate moiety attached to the anti-sense strand.

Heterocyclic base moieties (e.g., purines and pyrimidines) and monomeric subunits (e.g., sugar moieties), or monomeric subunit linkages (e.g., phosphodiester linkages) of nucleic acid molecules can present a linkage site. Conjugation to purines or purine derivatives can occur at any position including, endocyclic and exocyclic atoms. In some embodiments, the 2-, 6-, 7-, or 8-positions of a purine base are attached to a linking moiety. Conjugation to pyrimidines or derivatives thereof can also occur at any position. In some embodiments, the 2-, 5-, and 6-positions of a pyrimidine base can be substituted with a linking moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also serve as attachment points for linking moieties. For phosphorus-containing linkages (e.g., phosphodiester, phosphorothioate, phosphorodithiotate, phosphoroamidate, and the like), the linking moiety can be attached directly to the phosphorus atom or to an O, N, or S atom bound to the phosphorus atom. For amine- or amide-containing internucleosidic linkages (e.g., PNA), the linking moiety can be attached to the nitrogen atom of the amine or amide or to an adjacent carbon atom.

There are numerous methods described in the art for linking oligoribonucleotides, which generally comprise contacting a reactive group (e.g., OH, SH, amine, carboxyl, aldehyde, and the like) on the oligoribonucleotide compound with a reactive group on the linking moiety.

In some embodiments, one reactive group is electrophilic and the other is nucleophilic. For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative United States patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,672,662; 5,688,941; 5,714,166; 6,153,737; 6,172,208; 6,335,434; 6,335,437; 6,444,806; 6,486,308; 6,525,031; 6,528,631; 6,559,279; each of which is herein incorporated by reference.

The linkage between the multiple active agents (via a linking RNA or DNA or not) can, in certain embodiments, be via a functional nucleic acid such as an RNA or DNA aptamer. In other embodiments, the moiety is a lipid such as a fatty acyl, a glycerolipid, a glycerophospholipid, a sphingolipid, a sterol lipid, a prenol lipid, a saccharolipid, or a polyketide. In another embodiment, the moiety is a protein, such as an antibody, an enzyme, a receptor, or a ligand. In other embodiments, the moiety is a peptide, such as an affinity tag or a cell penetrating peptide. In preferred embodiments, the non-siRNA moiety is a polymer, such as a poly(ethylene glycol) (PEG), poly(propylene glycol) (PPG), poly(lactic acid) (PLA), poly(glycolic acid) (PGA), poly(lactic-co-glycolic acid) (PLGA), poly(hydroxyalkanoate) (PHA), poly(ethyleneimine) (PEI), a poly(beta-amino ester), polyvinyl alcohol, polyhydroxyethyl methacrylate, polyacrylamide, polyacrylic acid, polyethyloxazole, polyvinyl pyrrolidinone, and polysaccharides such as detran, chitosan, alginates, hyaluronic acid, or combinations thereof. In a preferred embodiment, the polymer is a PEG. In another preferred embodiment, the polymer is a subunit constituent of a polymer.

In its most common form PEG is a linear polymer terminated at each end with hydroxyl groups:

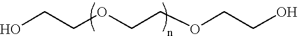

HO—CH2CH2O—(CH2CH2O)n-CH2-CH2OH

This polymer can be represented in brief form as HO-PEG-OH where it is understood that the —PEG-symbol represents the following structural unit:

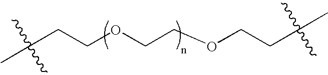

In typical form, n ranges from about 3 to about 4000, most typically from about 20 to about 2000. PEG having a molecular weight of about 200 Da to about 100,000 Da are particularly useful as the polymer in the present invention.

PEG is commonly used as methoxy-PEG-OH, or mPEG, in which one terminus is the relatively inert methoxy group, while the other terminus is a hydroxyl group that is subject to ready chemical modification.

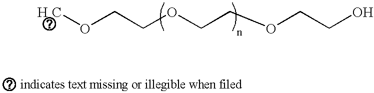

PEG is also commonly used in branched forms that can be prepared by addition of ethylene oxide to various polyols, such as glycerol, pentaerythritol and sorbitol. For example, the four-arm, branched PEG prepared from pentaerythritol is shown below:

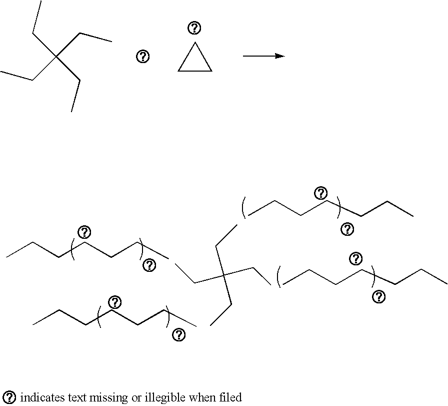

The branched polyethylene glycols can be represented in general form as R(—PEG-OH)ₙ in which R represents the central “core” molecule, such as glycerol or pentaerythritol, and n represents the number of arms.

In one embodiment, the two siRNAs are molecularly linked via a covalent bond. In some embodiments, the covalent bond is sufficiently stable such that once joined, the siRNA species are not expected to sever under physiological conditions taking place between formulation, delivery, internalization into target cell type(s), entry into the cytoplasm, and interaction with the endogenous siRNA machinery (e.g. the RISC complex). In other embodiments, the siRNAs are molecularly linked via a “reversible” covalent bond, such as an ester, a disulfide, or a beta-thiopropionate bond. In these embodiments, following formulation and delivery, one or more physiological stimuli will disrupt the covalent linkage. In some embodiments, the physiological stimulus is entry into or fusion with a cellular endosome, which is an acidic environment capable of reducing ester, disulfide, beta-thiopropionate, and other bonds. In some embodiments, an ether bond is hydrolyzed generating an alcohol and a carboxylic acid. In some embodiments, the reduced glutathione naturally present within the cytoplasm of target cells cleaves a disulfide linkage between the siRNAs and the polymer.

In some embodiments, the siRNAs are conjugated to a small molecule drug, a metabolite, a sugar, a polysaccharide, a lipid, a therapeutic peptide, a therapeutic protein (i.e. a recombinant protein, an antibody, etc), RNA or DNA. The nature and positioning of the conjugates are designed such that they do not reduce the RNAi activity of the siRNA species. In other embodiments, a modest reduction in activity is tolerated or desirable, for example, to minimize toxicity to bystander cells that are not in need of the siRNA therapy.

Methods to conjugate two molecular species are well in known in chemistry (Perlmutter, P, in Conjugate Addition Reactions in Organic Synthesis, Pergamon Press, New York, 1992). In certain embodiments, one of the species incorporates a “reactive group” which, under the appropriate chemical conditions, reacts with a functional group on the second species. The first species may include an N-hydroxysuccinimide ester (NHS-ester), an isocyanate, a 4-nitrophenyl carbonate or an aldehyde which can be coupled to an amine group on the second species. Alternatively or in addition, the first species may include a maleimide, an acrylate, a vinylsulfone, an orthopyridyl-disulfide or an iodoacetamide group which can be coupled with a thiol on the second species. Alternatively or in addition, a carboxylic acid group on the first species can be coupled to a hydroxyl group on the second species to generate an ester bond via an acid bromide (using phosphoryus tribromide) or acid chloride intermediate (using thionyl chloride).

Representative conjugate moieties can include lipophilic molecules (aromatic and non-aromatic) including steroid molecules; proteins (e.g., antibodies, enzymes, serum proteins); peptides; vitamins (water-soluble or lipidsoluble); polymers (water-soluble or lipid-soluble); small molecules including drugs, toxins, reporter molecules, and receptor ligands; carbohydrate complexes; nucleic acid cleaving complexes; metal chelators (e.g., porphyrins, texaphyrins, crown ethers, etc.); intercalators including hybrid photonucleaselintercalators; crosslinking agents (e.g., photoactive, redox active), and combinations and derivatives thereof. Numerous suitable conjugate moieties, their preparation and linkage to oligomeric compounds are provided, for example, in WO 93107883 and U.S. Pat. No. 6,395,492, each of which is incorporated herein by reference in its entirety. Oligonucleotide conjugates and their syntheses are also reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense & Nucleic Acid Drug Development, 2002, 12, 103, each of which is incorporated herein by reference in its entirety.

The terminal ends of polymers, such as PEG, can readily be functionalized to incorporate an activated derivative. Methods of functionalizing such polymers are well known to those in the art [see for example, U.S. Pat. Nos. 4,670,417 “Hemoglobin combined with a poly(alkylene oxide),” 6,828,401 “Preparation method for PEG-maleimide derivatives,” and 6,214,966 “Soluble, degradable poly(ethylene glycol) derivatives for controllable release of bound molecules into solution.”]. Reactive polymers can be prepared in house or are readily available from commercial sources, including but not limited to SunBio PEG-SHOP (Orinda, Calif.), Pierce (Rockford, Ill.), Jenkem Technologies USA (Allen, Tex.), Creative PEGWorks (Winston Salem, N.C.), Polysciences Inc (Warrington, Pa.), and Advanced Polymer Materials Inc (Montreal, Canada).

Methods of synthesizing or preparing amine or thiol derivatives of oligonucleotides are well known to those skilled in the art (see for example U.S. Pat. No. 6,114,513 “Thiol derivatized oligonucleotides,” U.S. Pat. No. 7,037,646 “Amine-derivatized nucleosides and oligonucleosides). 5′-, 3′-, and internally modified oligonucleotides (e.g. siRNA) are readily available from commercial sources including Integrated DNA Technologies (Coralville, Iowa) and Dharmacon (Lafayette, Colo.). They can also be prepared via standard phosphoramidite chemistry using the appropriately modified phosphoramidites (available from Glen Research, Sterling, Va.). In some embodiments, the reactive groups on the siRNA are separated by a 6-carbon linker. In other embodiments, the reactive groups on the siRNA are separated by a longer linker (e.g. 9, 12, 18-carbons and the like).

In some embodiments, the polymers are linear. A single linear polymer possesses two ends. Thus, in some embodiments, a linear polymer links two distinct siRNAs. In certain embodiments, the derivative linear polymers are homobifunctional, thus containing the same reactive group on both ends (e.g. maleimide[MAL]-PEG-MAL, or NHS-PEG-NHS). In other embodiments, the derivative polymers are heterobifunctional, thus containing two distinct reactive groups (e.g. MAL-PEG-NHS, or acrylate-PEG-isocyanate). In preferred embodiments, heterobifunctional polymers are employed such that one end is conjugated to an amine-modified first siRNA whereas the second end is conjugated to a thiol-modified second siRNA. Methods of preparing heterobifunctional polymers and their bioconjugation to entities have been described [WO 2004/085386 “Heterobifunctional polymeric bioconjugates”].

In other embodiments, the polymers are branched. Thus, in certain embodiments, a three-arm polymer links three distinct siRNA, a four-arm polymer links four distinct siRNAs, a six-arm polymer links six distinct siRNA, an eight-arm polymer links eight distinct siRNAs, a ten-arm polymer links ten distinct siRNAs, a twelve-arm polymer links twelve distinct siRNAs, and the like. In other embodiments, a multi-arm polymer (e.g. three, four, five, six, seven, eight, nine, ten, or more arms) links two or more distinct siRNAs but a fewer number of distinct siRNAs than total arms. For example, an eight-arm polymer links two, three, four, five, six, or seven siRNAs. In these embodiments, the branched polymers increase the valency of the therapeutic entity, increasing the dose and/or quantity of siRNA species delivered per cell. In certain embodiments, the termini may be homomultifunctional (e.g. all containing the same reactive group, such as acryalate) or heteromultifunctional (e.g. containing at least two distinct reactive groups, such as acrylate and NHS).

In another embodiment, the first agent (siRNA or non-siRNA) and second siRNA agents and are incorporated into polymers, such as poly(bet-amino esters). Methods to formulate and produce such polymers are well known to those skilled in the art (see Langer R et al, WO 2004/106411 “Biodegradable poly(beta-amino esters) and uses thereof. Although these polymers have previously been used to deliver nucleic acids (via encapsulation), the present invention for the first time discloses methods of directly and molecularly linking a first agent and a second siRNA agent into said polymers. In one embodiment, two agents of the present invention include a primary amine, and are mixed with a bis(acrylate) ester, forming a polymeric poly(beta-amino ester). In other embodiments, two agents of the present invention include a bis(secondary amine) which are condensed with bis(acrylate) to form a poly(beta-amino ester). Non-limiting examples of suitable di-acrylates[bis(acrylates)] are shown in Table 2.

TABLE 2

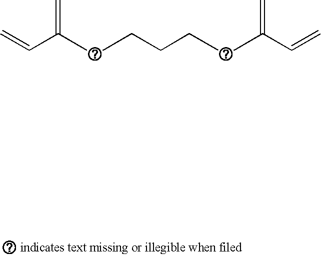

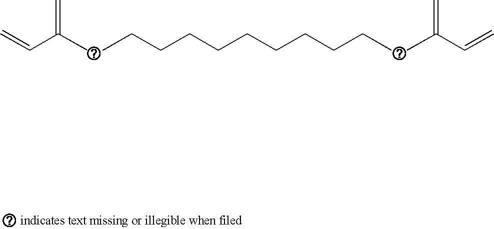

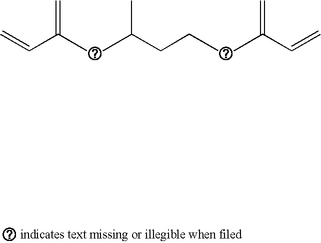

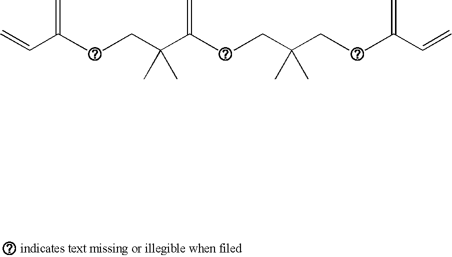

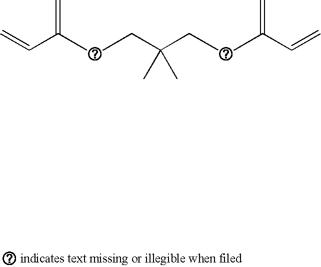

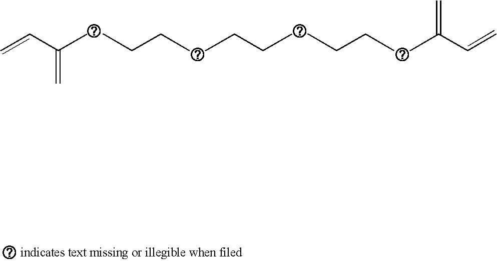

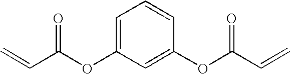

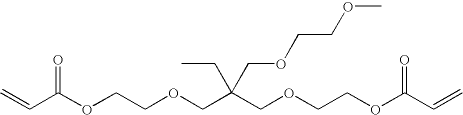

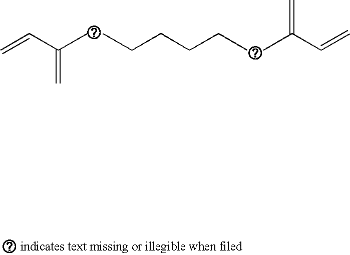

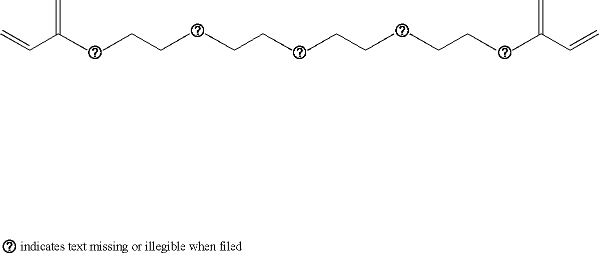

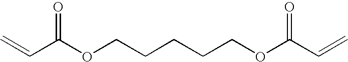

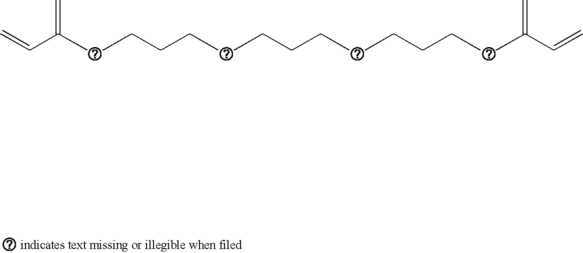

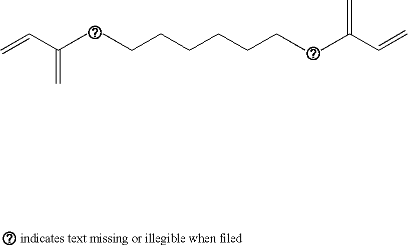

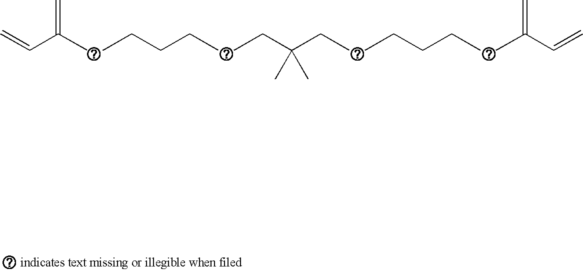

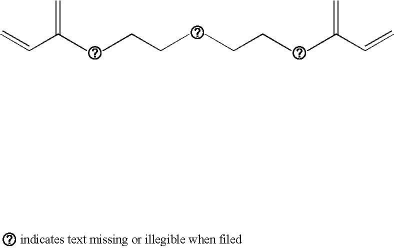

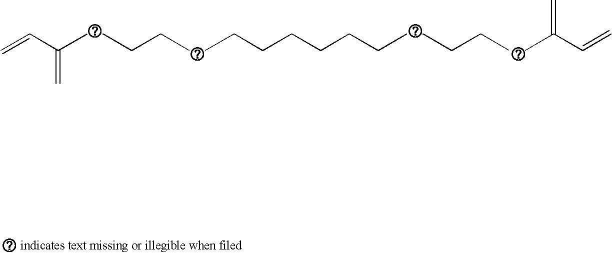

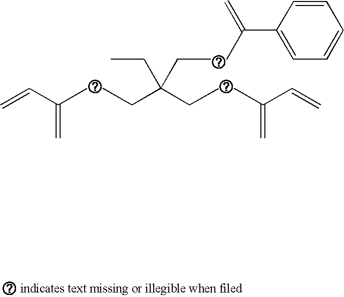

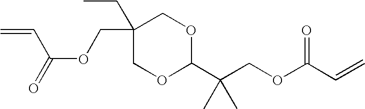

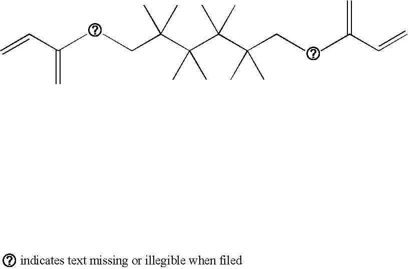

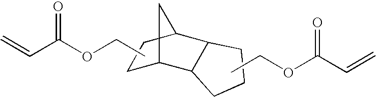

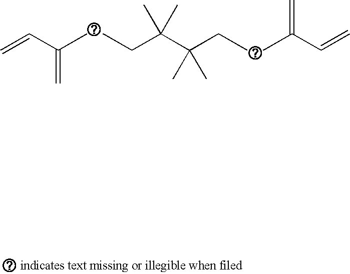

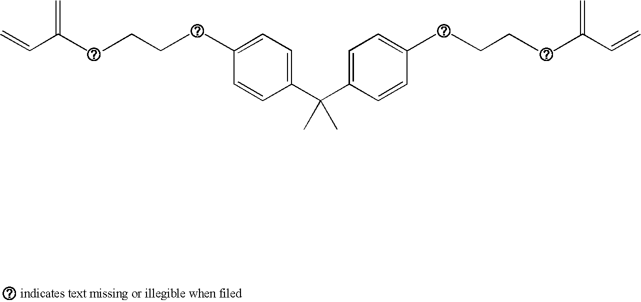

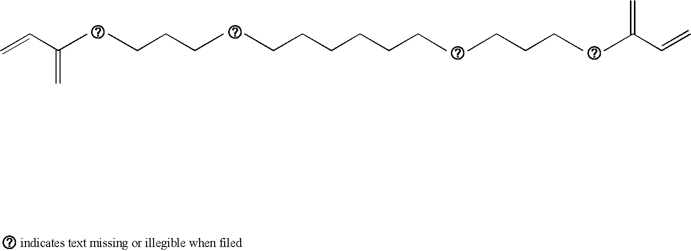

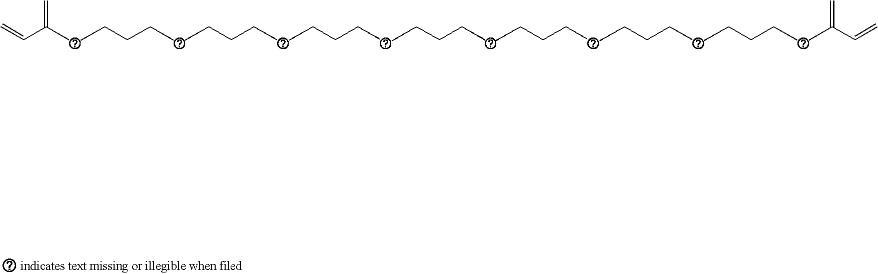

Non-limiting examples of amines are shown in Table 3.

TABLE 3

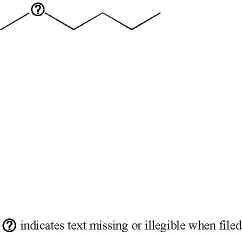

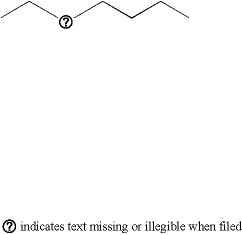

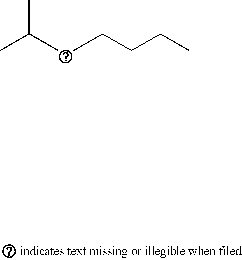

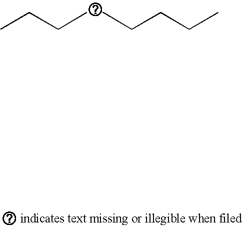

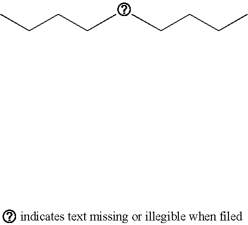

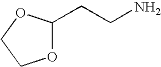

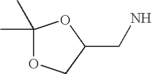

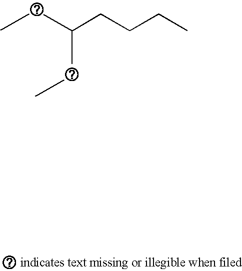

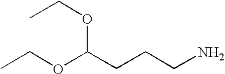

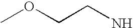

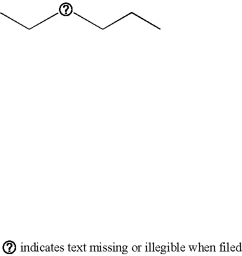

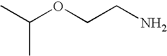

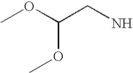

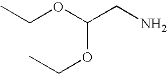

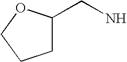

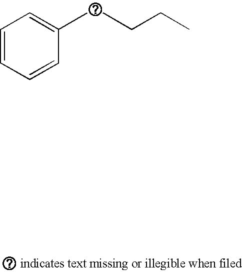

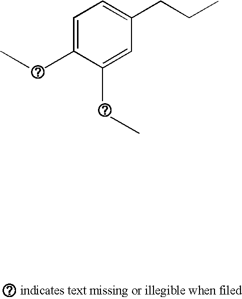

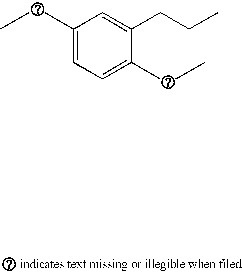

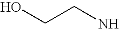

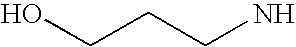

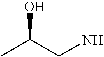

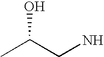

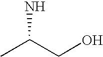

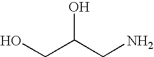

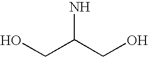

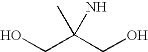

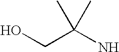

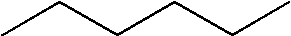

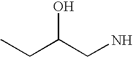

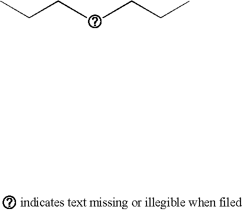

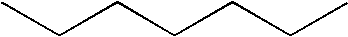

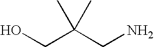

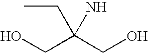

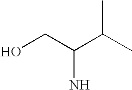

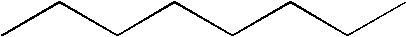

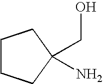

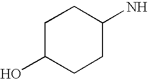

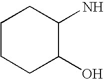

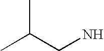

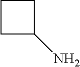

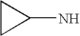

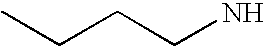

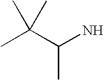

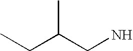

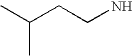

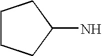

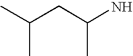

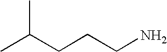

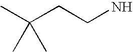

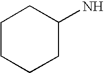

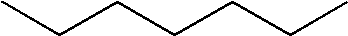

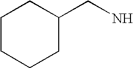

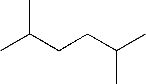

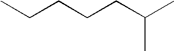

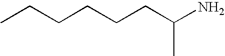

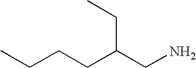

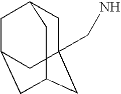

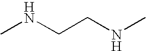

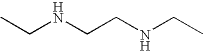

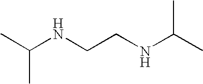

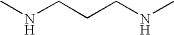

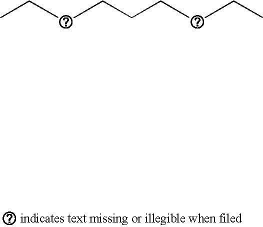

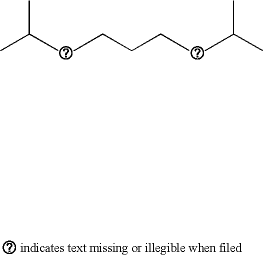

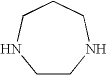

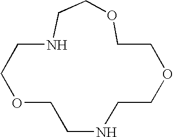

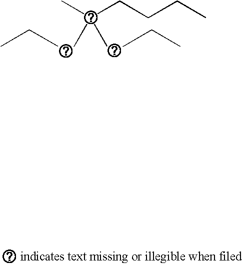

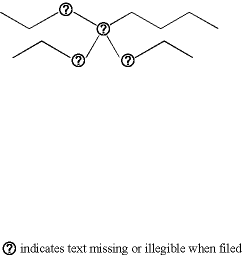

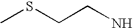

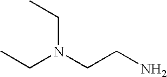

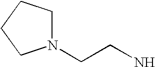

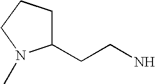

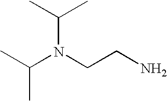

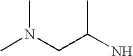

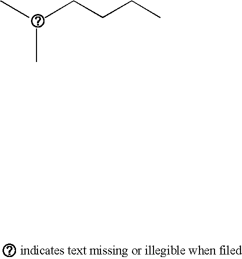

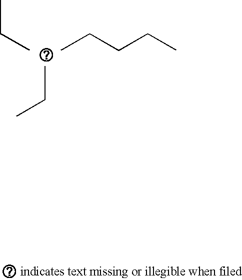

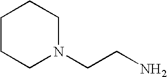

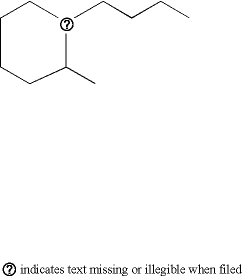

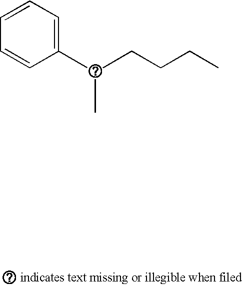

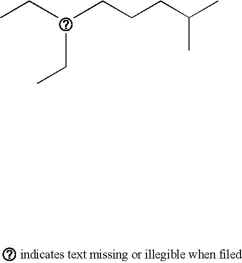

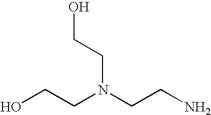

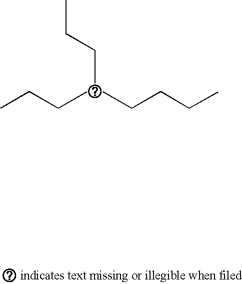

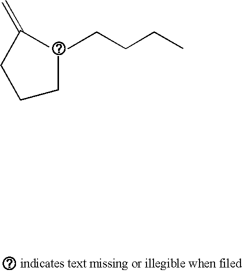

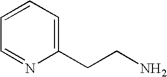

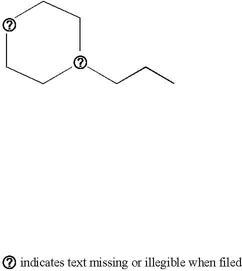

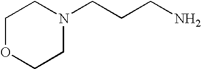

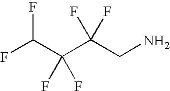

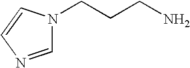

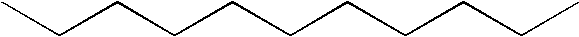

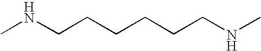

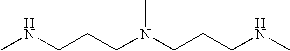

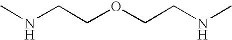

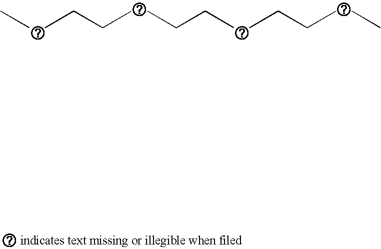

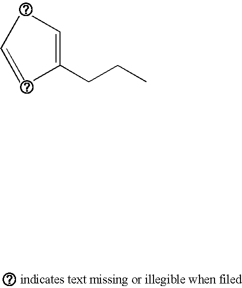

In some embodiments, said amines are copolymerized with amine-modified agents of the present invention and bis(acrylates). In other embodiments, the first or second agent is first linked to one or more of the above amine groups using methods described herein or well known to those skilled in the art. For example, a phosphoramidite building block can be synthesized such that the amine linker is directly incorporated into an siRNA agent at the 5′ or 3′ end. Alternatively or in addition, a reactive functional group on the first agent and/or the second agent can be reacted with a function group (e.g. a —OH) on one of the aforementioned amines. In some embodiments, the polymers are formed via biphasic synthesis (e.g. water in hexane).

Other cationic lipids can be similarly used. Suitable cationic lipid species include, but are not limited to: 3β[⁴N—(¹N,⁸N-diguanidino spermidine)-carbamoyl]cholesterol (BGSC); 3β[N,N-diguanidinoethyl-aminoethane)-carbamoyl]cholesterol (BGTC); N,N¹,N²,N³ Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2-(sperminecarboxamido)ethyl]-N,N-dimethyl-1-propanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3-dioleoyloxy-1,4-butanediammonium iodide) (Tfx-50); 1,2bis(oleoyloxy)-3-(trimethylammonio) propane (DOTAP); N-1-(2,3-dioleoyloxy) propyl-N, N,N-trimethyl ammonium chloride (DOTMA) or other N—(N,N-1-dialkoxy)-alkyl-N,N, N-trisubstituted ammonium surfactants; 1,2 dioleoyl-3-(4′-trimethylammonio) butanol-sn-glycerol (DOBT) or cholesteryl (4′ trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-.beta.-hydroxyethylammonium) or DORIE (DL-1,2-O-dioleoyl-3-dimethylaminopropyl-.beta.-hydroxyethylammonium) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 5,283,185, cholesteryl-3.beta.-carboxyl-amido-ethylenetrimethyl-ammonium iodide, 1-dimethylamino-3-trimethylammonio-DL-2-propyl-cholesteryl carboxylate iodide, cholesteryl-3β-carboxyamidoethyleneamine, cholesteryl-3β-oxysuccinamido-ethylenetrimethylammonium iodide, 1-dimethylamino-3-trimethylammonio-DL-2-propyl-cholesteryl-3,β-oxysuccinate iodide, 2-(2-trimethylammonio)-ethylmethylamino ethyl-cholesteryl-3β-oxysuccinate iodide, 3β-N—(N′,N′-dimethylaminoethane) carbamoyl cholesterol (DC-chol), and 3β-N-(polyethyleneimine)-carbamoylcholesterol. Examples of preferred cationic lipids include N-t-butyl-N′-tetradecyl-3-tetradecyl-aminopropion-amidine (CLONfectin), 2,3-dioleoyloxy-N-[2-(sperminecarboxamido)ethyl]-N,N-dimethyl-1-propanaminium trifluoroacetate (DOSPA), 1,2-bis(oleoyloxy)-3-(trimethylammonio)propane (DOTAP), N-[1-(2,3-dioleoyloxy)propyl]-N,N,N-trimethylammonium chloride) (DOTMA), cholesteryl-3β-carboxyamidoethylenetri-methylammonium iodide, 1-dimethylamino-3-trimethylammonio-DL-2-propyl-cholesteryl carboxylate iodide, cholesteryl-3β-carboxyamidoethyleneamine, cholesteryl-3β-oxysuccin-amidoethylenetrimethyl-ammonium iodide, 1-dimethylamino-3-trimethylammonio-DL-2-propyl-cholesteryl-3β-oxysuccinate iodide, 2-(2-trimethylammonio)ethylmethylamino ethyl-cholesteryl-3β-oxysuccinateiodide, 3β-N—(N′,N′-dimethyl-aminoethane)-carbamoylcholesterol (DC-chol), and 3βN-(polyethyleneimine)-carbamoyl cholesterol.

Bifunctional cross-linker molecules are also used. The cross-linker molecules may be homo-bifunctional or hetero-bifunctional, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulfhydryls, carboxyls, carbonyls and carbohydrates. Examples of amine-specific cross-linkers are bis(sulfosuccinimidyl) suberate, bis[2-(succinimidooxycarbonyloxy)ethyl]sulfone, disuccinimidyl suberate, disuccinimidyl tartarate, dimethyl adipimate.2HCl, dimethyl pimelimidate.2HCl, dimethyl suberimidate.2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide-2HCl, 4-(4-N-maleimidophenyl)-butyric acid hydrazide-2HCl, and 3-[2-pyridyldithio]propionyl hydrazide. The cross-linkers are bis-[β-4-azidosalicylamido)ethyl]disulfide and glutaraldehyde. Additionally, amine or thiol groups may be added to the molecules of the invention so as to provide a point of attachment for a bifunctional cross-linker molecule.

The complexes formed of the cationic polymer and active agents can be neutral. In other embodiments, the complexes are not neutral but are negatively or positively charged. The complexes include those with a positive zeta potential. The charge of the cationic polymer-active agent complexes is determined through the charge densities of the individual molecules as well as the amount of cationic polymer relative to the amount of polysaccharide (w/w) present to form the complex. In some embodiments the complexes have a net positive zeta potential. In other embodiments the complexes have a net negative zeta potential.

In another embodiment, the first agent (siRNA or non-siRNA) and second siRNA agents are incorporated into a polymer comprising PEG and PLGA. Methods of preparing PEG-PLGA diblock, triblock, and multiblock polymer are known to those skilled in the art (see for example Gref R et al, U.S. Pat. No. 5,565,215 “Biodegradable injectable particles for imaging,” and Shih et al, US Patent Application Number 2004/0185101 “Biodegradable triblock copolymers as solubilizing agents for drugs and methods of use thereof.”) Although these polymers have previously been used to deliver drugs or active agents, the present invention for the first time discloses methods of directly linking a first agent and a second siRNA agent into said polymers. In one embodiment, first agent (siRNA or non-siRNA) and second siRNA agents are first conjugated to a PEG species via linkages described herein or otherwise known to those skilled in the art. In some embodiments, the “pegylated” agents contain a free functional group (e.g. an amine, a thiol, a carboxylic acid, and the like) which are reacted with a suitable chemical constituent(s) at the termini of a PLGA polymer (e.g. PLGA containing an isocyanate, a succinimidyl succinate, an aldehyde, a maleimide, an orthopyridyl disulfide, a carboxylic acid and the like). In some embodiments, the linker chemistry between the first pegylated agent, the PLGA, and the second pegylated agent are the same. In other embodiments, the chemical linkers are distinct. In some embodiments, a defined “triblock” polymer is generated comprising the pegylated first agent (FA-PEG) and second agent (PEG-SA) conjugated to a central PLGA (FA-PEG-PLGA-PEG-SA). In other embodiments, a “multiblock” polymer is generated wherein multiple units of FA-PEG and PEG-SA are conjugated to PLGA. In some embodiments, the “pegylated” agents are polymerized with lactic acid and glycolic acid, for example via refluxing in toluene in the presence of stannous octoate. In some embodiments, the polymers generated are linear. In other embodiments the polymers are branched or star-shaped.

In some embodiments, the first agent (siRNA or non-siRNA) and second siRNA agents are further linked to a labeling agent, including but not limited to a nucleic acid, a radioisotope, peptide, a protein, a fluorophore, or a small molecule. In preferred embodiments, the labeling agents are distinct. In certain embodiments, the labeling agents are use to isolate or purify the desired molecular conjugate(s). Suitable yet non-limiting isolation techniques include ion exchange or high performance liquid chromatography, fluorescence-activated sorting, immunoprecipitation and affinity chromatography. In some embodiments, the isolation or purification procedures are used to purify conjugates containing both the first and second agent, either simultaneously or serially. In certain embodiments, the labeling agents are used to characterize, monitor or ensure the quality of the molecular conjugates. In still another embodiment, the labeling agents are used to monitor, detect or track the molecular conjugate within the context of a cell, animal, or human. In some embodiments, the labeling agents are also targeting agents, which influence the interaction of the molecular conjugates of the present invention with one or more peptides, proteins or lipids on or in a particular cell. In some embodiments, the labeling agent linked to the first agent and second agent are the same.

The foregoing list is meant to be illustrative and not limiting for the compounds which can be modified. Those of ordinary skill will realize that other such compounds or compositions can be similarly modified without undue experimentation. It is to be understood that linker chemistries not specifically mentioned but having suitable attachment groups are also intended and are within the scope of the present invention. There is no specific limit to the reaction conditions between the siRNA species and the non-siRNA moieties. Typically, during the formation of the conjugates, the ratio of siRNA to non-siRNA lies in range of 1:1000 to 1000:1. Similarly, the ratio of the individual siRNA species can be adjusted as desired. In some embodiments, the siRNAs are included at an equimolar ratio such that an equivalent amount of each siRNA is delivered to each target cell. In other embodiments, one or more siRNAs is included at a higher concentration relative to other siRNAs. Paramount to the utility of the present invention is the ability to consistently formulate a fixed molecular entity containing two or more agents.

In some embodiments, the conjugates of the present invention satisfactorily deliver their molecular payload to target cells in the absence of an additional carrier. In other embodiments, the conjugates are additionally formulated using siRNA delivery agents known to those skilled in the art including, but not limited to, liposomes, cationic liposomes, cationic polymers, targeting ligands, peptides, and antibodies. In most preferred embodiments, the conjugates of the present invention are formulated with cationic liposomes (e.g. 1,2-dioleyloxy-N,N-dimethyl-3-aminopropane or 1,2-dilinoleyloxy-N,N-dimethyl-3-aminopropane) or polymers (e.g. polyethylene imine, PEI).

In some embodiments of the present invention, the first agent is a non-siRNA biologically active compound, such as a chemotherapeutic, antibiotic, antiviral, anti-inflammatory, cytokine, immunotoxin, anti-tumor antibody, anti-angiogenic, anti-edema, or radiosensitizer agent. In some aspects of the present invention, the non-siRNA agent and the siRNA agent are directly or indirectly conjugated, for example via a polymer. The chemical structure of the first agent is not limited. Drugs can be used which have a functional group which can be bonded to polymers, such as PEG or PLGA. Specific examples of drugs are ones having hydroxyl, carboxyl, carbonyl, amino or alkenyl. The drugs can be converted into derivatives having a functional group such as amino, thiol, carboxyl or isothiocyanate in advance so that the covalent bond to the polymer can be easily formed. Various specific drug-PEG conjugates have been synthesized, and insulin-PEG conjugates (U.S. Pat. No. 4,179,337), taxol-PEG conjugates (WO 93/24476), interferon-PEG conjugates (WO 99/48535), asparaginase-PEG conjugates (WO 99/39732) and urate oxidase-PEG conjugates (WO 00/7629) are known. However, the rationale for the drug-PEG conjugates were limited to improved solubility and/or bioavailability, None of these approaches captured the inventive improvement comprising coupling a drug-polymer conjugate to an siRNA agent, to achieve additive or synergistic therapeutic improvements.

The complexes of cationic polymer and active provided herein also include complexes that are internalized rapidly and/or keep the active agent in the cell for a period of time. Methods for analyzing the internalization of the active agent into a cell are known in the art and are also provided below in the Examples. As used herein to be “internalized rapidly” means that the polymer-active agent conjugate is internalized within 1, 2, 3, 4, 5 or 6 hours. Still other complexes that are rapidly internalized are those that are internalized within less than 24 hours. Preferably, the complexes keep the active agent, once internalized, in a cell for more than 3, 4, 5, 6, 7, 8, 9, 10, 11, 12, 13, 14, 15, 16, 17, 18, 19, 20, 21, 22, 23, or 24 or more hours. Also preferred are complexes that cause the active agent to be delivered to the nucleus, cytosol, or other non-reticulo locations.

Additionally, in some embodiments the complexes provided herein have an “effective diameter.” As used herein the “effective diameter” of the complexes is one that allows for the internalization of a particular polysaccharide. In some embodiments, the effective diameter is less than 200 nm. In some embodiments, the effective diameter is 5 nm, 6, nm, 7, nm, 8, nm 9, nm, 10 nm, 20 nm, 30 nm, 40 nm, 50 nm, 60 nm, 75 nm, 100 nm, 150 nm, 175 nm or less. However, in other embodiments the effective diameter is greater than 200 nm. Particularly, in some embodiments, the effective diameter is 210 nm, 220 nm, 230 nm, 240 nm, 250 nm, 275 nm, 300 nm, 400 nm, 500 nm or more.

In some embodiments the polymers and/or active agents are in a substantially pure form. As used herein, with respect to these molecules, described herein, the term “substantially pure” means that the molecules of the invention are essentially free of other substances with which they may be found in nature or in vivo systems to an extent practical and appropriate for their intended use. In particular, the molecule is sufficiently free from other biological constituents of the host cells so as to be useful in, for example, producing pharmaceutical preparations. Because the molecules of the invention may be admixed with a pharmaceutically acceptable carrier in a pharmaceutical preparation, the molecule may comprise only a small percentage by weight of the preparation. The molecule is nonetheless substantially pure in that it has been substantially separated from the substances with which it may be associated in living systems. Active agents can be isolated from biological samples or can be synthesized using standard chemical synthetic methods. Polymers likewise can be isolated from biological samples or can be synthesized using standard chemical synthetic methods. Some polymers, such as proteins and peptides, can also be expressed recombinantly in a variety of prokaryotic and eukaryotic expression systems by constructing an expression vector appropriate to the expression system, introducing the expression vector into the expression system, and isolating the recombinantly expressed protein.

As used herein with respect to the molecules provided herein, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, i.e., isolated from other proteins.

In some of the compositions provided herein, the active agent is present in a therapeutically effective amount. As used herein, the active agent can have any of a number of therapeutic activities. For instance, in some embodiments the active agent is in a therapeutically effective amount to promote apoptosis. The term “therapeutically effective amount” also includes an amount of the active agent that inhibits cell growth. The therapeutically effective amount is, therefore, in some embodiments, such an amount that would be useful to inhibit or retard cell proliferation.

In one embodiment a therapeutically effective amount is an intracellular therapeutically effective amount. This term refers to the percentage of cells, to which an active agent in complexed or uncomplexed form has been placed in contact with, that contains (within the cell) the administered active agent. In one embodiment the intracellular therapeutically effective amount is when greater than 5%, 10%, 15%, 20%, 25%, 30%, 35%, 40%, 45%, 50%, 55%, 60%, 65%, 70%, 75%, 80%, 85%, 90%, 95% or more of the cells contacted with the complexed or uncomplexed active agent contain the active agent. As one non-limiting example the intracellular therapeutically effective amount is when greater than 20%, 25%, 50%, 75%, 90%, 95% or more of the cells of a tumor contain the administered active agent.

Compositions are also provided that comprise an active agent in uncomplexed form (i.e., not complexed to a cationic polymer and/or not associated with any molecule) and in an intracellular therapeutically effective amount. The active agent can be any of the active agents described herein.

The compositions provided can also be a solution. In one embodiment the solution has a physiological pH. In another embodiment the composition can further contain a pharmaceutically acceptable or physiologically acceptable carrier. In still another embodiment the composition can contain sodium acetate and/or PBS.

Compounds of Interest for the Treatment of Disease through RNAco-i

The compositions and methods provided have not only uses in vitro but also in vivo, such as for a number of therapeutic purposes. The compositions of the invention can be used for the treatment of any condition in which two or more selected agents for RNAco-i allow for an enhanced phenotypic effect.

In another aspect of the invention screening methods are provided whereby the RNAco-i agents provided can be used to screen for RNAco-i inhibiting agents. In such methods the candidate RNAco-i inhibiting agents are contacted with cells in the presence of an RNAco-i agent. The method further includes the step of evaluating whether or not the candidate agent inhibited the disruption of the intercellular junctions by the RNAco-i agent. Compositions and methods are further provided using these discovered RNAco-i inhibiting agents.

In some embodiments, the compositions of RNAco-i agents can be administered to a subject “not ordinarily in need of treatment thereof” A subject not ordinarily in need of treatment thereof refers to a subject who suffers from a condition where the RNAco-i agent is not normally administered to treat the condition. Conditions which are not ordinarily treated with RNAco-i agents can include in some embodiments nonangiogenic, noncoagulation, nonthrombotic, nonrespiratory, noninflammatory, nonimmunologic, nonallergic and/or nonvascular disorders. In some embodiments, depending on the RNAco-i agent, the condition is not a neurodegenerative disease and/or not a central nervous system disorder. In some embodiments the condition is not spinal cord injury. In other embodiments the subject is not in need of neural regeneration. In some embodiments the condition is not Alzheimers. In some embodiments the condition is a central nervous system disorder that is not Alzheimers. In other embodiments the condition is not a dermatological disorder. In other embodiments the condition is not psoriasis. In some embodiments the condition is a dermatological disorder that is not psoriasis. In some embodiments the subject does not have a circulatory shock or related disorder, cardiovascular disease, atherosclerosis, cancer, stroke and/or Alzheimers. In some embodiments the subject has a condition that is not inflammatory bowel disease (e.g., Crohn's, ulcerative colitis). In some embodiments the condition is not a respiratory disorder. In other embodiments the subject does not have asthma, fibrotic lungs and/or an infection or an infection related disorder. In still other embodiments the subject has a condition that is a respiratory disorder that is not asthma. In some embodiments the subject does not have a pseudomonas infection or a S. aureus infection. In other embodiments the subject has an infection that is not a pseudomonas infection or a S. aureus infection. In some embodiments the condition is not an inflammatory disorder. In some embodiments the condition is not an immunologic disorder. In other embodiments the condition is not lupus. In some embodiments the condition is an immunologic disorder that is not lupus. In other embodiments the subject is not undergoing a tissue or organ transplant or a surgical procedure where the use of the RNAco-i agent would be normally desired.

As stated above the compositions and methods provided herein can be used, depending on the RNAco-i agent, to treat and/or prevent a number of disorders. As used herein, disease and disorder are used interchangeably.

The primary active agent used in RNAco-i is an agent that confers RNAi. In a preferred embodiment, the agent is a siRNA. In preferred embodiments, the siRNA is 19, 20, 21, 22, 23, 24, 25, 26, 27, 28, 29, 30, 31, 32, 33, 34, or 35 nucleotides long. It is obvious to one who is skilled in the art that forming an equally or otherwise similarly effective siRNA at an alternative length could equally be used. Correspondingly, RNAi has been conferred by means with much longer-up to 714-nucleotide lengths Li et al., International PCT Publication No. WO 00/44914; Zernicka-Goetz et al., International PCT Publication No. WO 01/36646. In other embodiments, RNAi can be conferred by shRNA, miRNA, and diRNA. In other embodiments, the RNAco-i agent is a vector (e.g. a virus, or a plasmid) that directs the target cell to produce RNAco-i agent(s), such as shRNA, miRNA, or siRNA.

In some embodiments, the compositions are useful for treating or preventing disorders associated with coagulation. A “disease associated with coagulation” as used herein refers to a condition characterized by local inflammation resulting from an interruption in the blood supply to a tissue due to a blockage of the blood vessel responsible for supplying blood to the tissue such as is seen for myocardial or cerebral infarction. Coagulation disorders include, but are not limited to, cardiovascular disease and vascular conditions such as cerebral ischemia. The compositions and methods of the invention are also useful for treating cardiovascular disease. Cardiovascular diseases include, but are not limited to, acute myocardial infarction, unstable angina and atrial fibrillation.

The compositions and methods provided thus can also include anti-inflammatory agents, anti-thrombotic agents, anti-platelet agents, fibrinolytic agents, lipid reducing agents, direct thrombin inhibitors, and glycoprotein IIb/IIIa receptor inhibitors.

Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium.

Lipid reducing agents include gemfibrozil, cholystyramine, colestipol, nicotinic acid, probucol lovastatin, fluvastatin, simvastatin, atorvastatin, pravastatin, cirivastatin.

Glycoprotein IIb/IIIa receptor inhibitors are both antibodies and non-antibodies, and include but are not limited to ReoPro (abcixamab), lamifiban, tirofiban.

Anti-thrombotic agents and anti-platelet agents are described in more detail below.

The compositions provided are also useful for treating vascular conditions. Vascular conditions include, but are not limited to, disorders such as deep venous thrombosis, cerebral ischemia, including stroke, and pulmonary embolism. Because it is often difficult to discern whether a stroke is caused by a thrombosis or an embolism, the term “thromboembolism” is used to cover strokes caused by either of these mechanisms. The compositions can also be very valuable in the treatment of venous thromboembolism. The methods of the invention in some embodiments are directed to the treatment of acute thromboembolic stroke. An acute stroke is a medical syndrome involving neurological injury resulting from an ischemic event, which is an interruption in the blood supply to the brain.

Compositions and methods, therefore, are also provided to treat a cerebrovascular condition. Cerebrovascular conditions include, for example, stroke, cerebral arteriosclerosis, cerebral aneurysm, intracranial hemorrhage (subarachnoid hemorrhage, berry aneurysms etc.), lacunar infarcts, slit hemorrhages (hypertension related), hypertensive encephalopathy, cerebral autosomal dominant arteriopathy with subcortical infarcts and leukoencephalopathy (CADASIL), and cerebral artery disease. The compositions and methods provided can also be used to treat brain injury and/or enhance brain function such as enhancing cerebral/behavioral function.

An effective amount of the compositions provided for the treatment of stroke is that amount sufficient to reduce in vivo brain injury resulting from the stroke. A reduction of brain injury is any prevention of injury to the brain which otherwise would have occurred in a subject experiencing a thromboembolic stroke absent the treatment of the invention. Several physiological parameters may be used to assess reduction of brain injury, including smaller infarct size, improved regional cerebral blood flow, and decreased intracranial pressure, for example, as compared to pretreatment patient parameters, untreated stroke patients or stroke patients treated with thrombolytic agents alone.

The compositions provided may be used for treating a disease associated with coagulation. Examples of therapeutics useful in the treatment of diseases associated with coagulation include anticoagulation agents, antiplatelet agents, and thrombolytic agents.

Anticoagulation agents prevent the coagulation of blood components and thus prevent clot formation. Anticoagulants include, but are not limited to, warfarin, coumadin, dicumarol, phenprocoumon, acenocoumarol, ethyl biscoumacetate, and indandione derivatives.

Antiplatelet agents inhibit platelet aggregation and are often used to prevent thromboembolic stroke in patients who have experienced a transient ischemic attack or stroke. Antiplatelet agents include, but are not limited to, aspirin, thienopyridine derivatives such as ticlopodine and clopidogrel, dipyridamole and sulfinpyrazone, as well as RGD mimetics and also antithrombin agents such as, but not limited to, hirudin.

Thrombolytic agents lyse clots which cause the thromboembolic stroke. Thrombolytic agents have been used in the treatment of acute venous thromboembolism and pulmonary emboli and are well known in the art (e.g. see Hennekens et al, J Am Coll Cardiol; v. 25 (7 supp), p. 18S-22S (1995); Holmes, et al, J Am Coll Cardiol; v. 25 (7 suppl), p. OS-17S(1995)). Thrombolytic agents include, but are not limited to, plasminogen, a₂-antiplasmin, streptokinase, antistreplase, tissue plasminogen activator (tPA), and urokinase. “tPA” as used herein includes native tPA and recombinant tPA, as well as modified forms of tPA that retain the enzymatic or fibrinolytic activities of native tPA. The enzymatic activity of tPA can be measured by assessing the ability of the molecule to convert plasminogen to plasmin. The fibrinolytic activity of tPA may be determined by any in vitro clot lysis activity known in the art, such as the purified clot lysis assay described by Carlson, et. al., Anal. Biochem. 168, 428-435 (1988) and its modified form described by Bennett, W. F. Et al., 1991, Supra, the entire contents of which are hereby incorporated by reference.

Pulmonary embolism as used herein refers to a disorder associated with the entrapment of a blood clot in the lumen of a pulmonary artery, causing severe respiratory dysfunction. Pulmonary emboli often originate in the veins of the lower extremities where clots form in the deep leg veins and then travel to lungs via the venous circulation. Thus, pulmonary embolism often arises as a complication of deep venous thrombosis in the lower extremity veins. Symptoms of pulmonary embolism include acute onset of shortness of breath, chest pain (worse with breathing), and rapid heart rate and respiratory rate. Some individuals may experience haemoptysis.

The products and methods of the invention are also useful for treating or preventing atherosclerosis. Atherosclerosis is one form of arteriosclerosis that is believed to be the cause of most coronary artery disease, aortic aneurysm and atrial disease of the lower extremities, as well as contributing to cerebrovascular disease.

The compositions provided are also valuable in treatment of respiratory diseases such as asthma, allergic disorder, emphysema, adult respiratory distress syndrome (ARDS), lung reperfusion injury, ischemia-reperfusion injury of the lung, kidney, heart, and gut, and lung tumor growth and metastasis. Asthma is a disorder of the respiratory system characterized by inflammation, narrowing of the airways and increased reactivity of the airways to inhaled agents. Asthma is frequently, although not exclusively, associated with atopic or allergic symptoms. Asthma may also include exercise induced asthma, bronchoconstrictive response to bronchostimulants, delayed-type hypersensitivity, auto immune encephalomyelitis and related disorders. Allergies are generally caused by IgE antibody generation against allergens. Emphysema is a distention of the air spaces distal to the terminal bronchiole with destruction of alveolar septa. Emphysema arises out of elastase induced lung injury. Adult respiratory distress syndrome is a term which encompasses many acute defuse infiltrative lung lesions of diverse ideologies which are accompanied by severe atrial hypoxemia. One of the most frequent causes of ARDS is sepsis. Other types of inflammatory diseases which are treatable with the compositions provided are refractory ulcerative colitis, non-specific ulcerative colitis and interstitial cystitis.

The compositions and methods provided are also useful for treating lung disease, such as chronic obstructive pulmonary disease/disorder (COPD), fibrosis, restrictive lung disease, mesothelioma, pneumonia, sarcoidosis and cystic fibrosis.

The compositions can also be used for inhibiting angiogenesis. Angiogenesis as used herein is the inappropriate formation of new blood vessels. “Angiogenesis” often occurs in tumors when endothelial cells secrete a group of growth factors that are mitogenic for endothelium causing the elongation and proliferation of endothelial cells which results in a generation of new blood vessels. The inhibition of angiogenesis can cause tumor regression in animal models, suggesting a use as a therapeutic anticancer agent. An effective amount for inhibiting angiogenesis is an amount which is sufficient to diminish the number of blood vessels growing into a tumor. This amount can be assessed in an animal model of tumors and angiogenesis, many of which are known in the art. Angiogenic disorders include, but are not limited to, neovascular disorders of the eye, osteoporosis, psoriasis and arthritis.

The compositions are also useful for inhibiting neovascularization associated with eye disease.

In another preferred embodiment, the composition is administered to treat psoriasis. Psoriasis is a common dermatologic disease causes by chronic inflammation.

The compositions for the treatment of psoriasis include, but are not limited to salicylic acid, coal tar, moisturizing agents, topical corticosteroids, http:///Anthralin, Dovonex (synthetic vitamin D3), Taclonex (synthetic vitamin D3 plus the steroid betamethasone dipropionate), Tazorac (vitamin A derivative, a topical retinoid), cyclosporine, methotrexate, Etanercept (“Enbrel”), and Infliximab (“Remicade”).

The compositions and methods provided are also useful for treating dermatological disorders. In some embodiments the dermatological disorder is not psoriasis. Dermatological disorders include vitiligo, melanoma, dysplasic nevi, seborrheic keratoses, acanthosis nigricans, adnexal tumors, other epidermal tumors (actinic keratosis, squamous cell carcinoma, basal cell carcinoma, merkel cell carcinoma, histiocytosis X, mycosis fungoides/cutaneous T-cell lymphoma), mastocytosis, eczema/acute eczematous dermatitis, urticaria, erythema multiforme, psoriasis, lichen planus, lupus/systemic lupus erythematosus, bussous diseases, acne vulgaris, and panniculitis.

The compositions may also inhibit cancer cell growth, reduce tumor size, prevent invasiveness, inhibit cancer progression and inhibit metastasis. Thus the methods of the invention are useful for treating tumor cell proliferation or metastasis in a subject. The terms “treat” and “treating” as used herein refer to inhibiting completely or partially the biological effect, e.g., angiogenesis or proliferation or metastasis of a cancer or tumor cell, as well as inhibiting any increase in the biological effect, e.g., angiogenesis or proliferation or metastasis of a cancer or tumor cell.

The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreatic cancer; prostate cancer; rectal cancer; sarcomas; skin cancer; testicular cancer; thyroid cancer; and renal cancer, glioblastoma, as well as other carcinomas and sarcomas.

A subject in need of treatment may be a subject who has a high probability of developing cancer. These subjects include, for instance, subjects having a genetic abnormality, the presence of which has been demonstrated to have a correlative relation to a higher likelihood of developing a cancer and subjects exposed to cancer-causing agents such as tobacco, asbestos, or other chemical toxins, or a subject who has previously been treated for cancer and is in apparent remission.

Anti-cancer drugs that can serve as biologically active molecules are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Anti-cancer agents can also include cytotoxic agents and agents that act on tumor neovasculature. Cytotoxic agents include cytotoxic radionuclides, chemical toxins and protein toxins. The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Toxins also include poisonous lectins, plant toxins such as ricin, abrin, modeccin, botulina and diphtheria toxins. Of course, combinations of the various toxins are also provided thereby accommodating variable cytotoxicity. Other chemotherapeutic agents are known to those skilled in the art.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein), interferon inducible protein 10 (U.S. Pat. No. 5,994,292), and the like. Anticancer agents also include immunomodulators such as α-interferon, γ-interferon and tumor necrosis factor alpha (TNFα).

The invention also contemplates compositions and methods for the treatment of subjects having or at risk of developing neurodegenerative disease or suffering an injury to nerve cells. Neuronal cells are predominantly categorized based on their local/regional synaptic connections (e.g., local circuit interneurons vs. longrange projection neurons) and receptor sets, and associated second messenger systems. Neuronal cells include both central nervous system (CNS) neurons and peripheral nervous system (PNS) neurons. There are many different neuronal cell types. Examples include, but are not limited to, sensory and sympathetic neurons, cholinergic neurons, dorsal root ganglion neurons, proprioceptive neurons (in the trigeminal mesencephalic nucleus), ciliary ganglion neurons (in the parasympathetic nervous system), c-fibers (pain fibers) etc. A person of ordinary skill in the art will be able to easily identify neuronal cells and distinguish them from non-neuronal cells such as glial cells, typically utilizing cell-morphological characteristics, expression of cell-specific markers, secretion of certain molecules, etc.

“Neurodegenerative disease/disorder” is defined herein as a disorder in which progressive loss of neurons occurs either in the peripheral nervous system or in the central nervous system. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor) and Wernicke-Korsakoff's related dementia. Neurodegenerative diseases affecting sensory neurons include Friedreich's ataxia, diabetes, peripheral neuropathy and retinal neuronal degeneration. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

The compositions provided, therefore, can include biologically active molecules for the promotion of nerve regeneration and/or treatment of neurodegenerative disease.

The biologically active molecules, therefore, can be antiparkinsonian agents, which include, for example, Benztropine Mesylate; Biperiden; Biperiden Hydrochloride; Biperiden Lactate; Carmantadine; Ciladopa Hydrochloride; Dopamantine; Ethopropazine Hydrochloride; Lazabemide; Levodopa; Lometraline Hydrochloride; Mofegiline Hydrochloride; Naxagolide Hydrochloride; Pareptide Sulfate; Procyclidine Hydrochloride; Quinelorane Hydrochloride; Ropinirole Hydrochloride; Selegiline Hydrochloride; Tolcapone; Trihexyphenidyl Hydrochloride. Drugs for the treatment of amyotrophic lateral sclerosis include but are not limited to Riluzole. Drugs for the treatment of Paget's disease include but are not limited to Tiludronate Disodium.

Biologically active molecules can also be agents that promote neuronal regeneration. Neuronal regenerative agents include growth factors and neurotrophic agents that promote neuronal growth and/or survival. Such examples include, but are not limited to, nerve growth factor (NGF), brain-derived neurotrophic factor (BDNF), cardiotrophin-1 (CT-1), choline acetyltransferase development factor (CDF), ciliary neurotrophic factor (CNTF) fibroblast growth factor-1 (FGF-1), FGF-2, FGF-5, glial cell-line-derived neurotrophic factor (GDNF), insulin, insulin-like growth factor-1 (IGF-1), IGF-2, interleukin-6 (IL-6), leukemia inhibitor factor (LIF), neurite promoting factor (NPF), neurotrophin-3 (NT-3), NT-4, platelet-derived growth factor (PDGF), protease nexin-1 (PN-1), S-100, transforming growth factor.beta. (TGF-.beta.), decorin, anti-TGF-beta antibodies, mutated TGF-beta, and vasoactive intestinal peptide (VIP) (Oppenheim, 1996, Neuron 17:195-197).

The compositions and methods provided can also be used to treat a subject with a central nervous system disorder. Central nervous system disorders include, for example, Alzheimers, Parkinson's disease, Huntington's disease, cerebrovascular disease, epilepsy, depression, mania, schizophrenia and psychotic disorders.

The compositions provided, therefore, can include agents for treating and/or preventing central nervous system disorders. Such agents include the following examples.

Benzodiazepines (e.g., Alprazolam, Chlordiazepoxide, Clorazepate, Clonazepam, Diazepam, Estazolam, Flurazepam, Halazepam, Lorazepam, Midazolam, Oxazepam, Prazepam, Quazepam, Temazepam, Triazolam); Benzodiazepine Antagonist (e.g., Flumazenil); Barbiturates (e.g., Amobarbital, Aprobarbital, Butabarbital sodium, Mephobarbital, Pentobarbital, Phenobarbital, Secobarbital); Buspirone; Chloral Hydrate; Ethchlorvynol; Ethinamate; Hydroxyzine; Meprobamate; Paraldehyde; Zaleplon; Zolpidem; Treatments of acute alcohol withdrawal syndrome (e.g., Clorazepate, Diazepam, Oxazepam, Thiamine); Treatments for the prevention of alcohol abuse (e.g., Disulfuram, Naltrexone); Treatments of acute methanol or ethylene glycol poisoning (e.g., Ethanol, Fomepizole); Anti-Epileptic Drugs (e.g., Carbamazepine, Clonazepam, Clorazepate Dipotassium, Diazepam, Ethusuximide, Ethotoin, Felbamate, Fosphenytoin, Gabapentin, Lamotrigine, Levetiracetam, Lorazepam, Mephenyloin, Mephobarbital, Methsuximide, Oxycarbapazepine, Paramethadione, Pentobarbital, Phensuximide, Phenyloin, Primidone, Tiagabine, Topiramate, Trimethadione, Valproic Acid); General Anesthetics (e.g., Desflurane, Demadetomidine, Diazepam, Enflurane, Etomidate, Halothane, Isoflurane, Ketamine, Lorazepam, Mthohexital, Mrthoxyflurane, Midazolam, Nitrous Oxide, Propofol, Sevoflurane, Thiamylal, Thiopental); Local Anesthetics (e.g., Benzocaine, Bupivacaine, Butamben Pictate, Choloprocaine, Cocaine, Dibucaine, Dyclonine, Etidocaine, Levobupivacaine, Lidocaine, Mepivacaine, Pramoxine, Prilocaine, Procaine, Proparacaine, Propoxicaine, Ropivacaine, Tetracaine); Skeletal Muscle Relaxants (e.g., Neuromuscular Blocking Agents: Atracurium, Cisatracurium, Doxacurium, Metocurine, Mivacurium, Pancuronium, Pipecuronium, Rapacuronium, Rocurinium, Succunylcholine, Tubocurarine, Vecuronium); Spasmolytics (e.g., Baclofen, Botulinum Toxin Type A, Carisoprodol, Chlorphenesin, Chlorzoxazone, Cyclobenzaprine, Diazepam, Gabapentin, Metaxalone, Methocarbamol, Orphenadrine, Riluzole, Tizanidine); Anti-Parkinsonism Agents (also movement disorder agents) (e.g., Amantadine, Benztropine, Biperiden, Bromocriptine, Carbidopa, Entacapone, Levodopa, Orphenadrine, Penicillamine, Pergolide, Pramipexole, Procyclidine, Ropinirole, Selegiline, Tolcapone, Trientine, Trihexyphenidyl); Antipsychotic Agents (e.g., Acetophenazine, Chlorpromazine, Chlorprothixene, Clozapine, Fluphenazine (& esters), Haloperidol (& esters), Loxamine, Mesoridazine, Molindone, Olanzapine, Perphenazine, Pimozide, Prochlorperazine, Promazine, Quetiapine, Risperidone, Sertindole, Thioridazine, Thiothixene, Trifluoperazine, Triflupromazine, Ziprasidone); Mood Stabilizers (e.g., Carbamazepine, Divalproex, Lithium, Valproic Acid); Anti-Depressant Agents (e.g., Tricyclics: Amitriptyline, Clomipramine, Desipramine, Doxepin, Imipramine, Nortryptyline, Protryptyline, Trimipramine); Second & Third Generation Agents (e.g., Amoxapine, Bupropion, Maprotiline, Mirtazapine, Nefazodone, Trazodone, Venlafaxine); Selective Serotonin Reuptake Inhibitors (e.g., Citalopram, Flouxetine, Fluvoxamine, Paroxetine, Sertraline); Monoamine Oxidase Inhibitors (e.g., Phenelzine, Tranylcypromine); Opioid Analgesics & Antagonists; Opioid Analgesics (e.g., Alfentanil, Buprenorphine, Butorphanol, Codeine, Dezocine, Fentanyl, Hydromorphone, Levomethadyl Acetate, Levorphanol, Meperidine, Methadone, Morphine, Nalbuphine, Oxycodone, Oxymorphone, Pentazocine, Propoxyphene, Remifentanil, Sufentanil, Tramadol); Opioid Antagonists (e.g., Nalmefene, Naloxone, Naltrexone); and Antitussives (e.g., Codeine, Dextromethorphan).

The compositions provided herein can also be used for the treatment of rheumatoid arthritis, osteoarthritis or psoriasis. Treatment of osteoarthritis refers to any reduction of the subject's symptoms associated with osteoarthritis or controlling the progression of the disease. Generally treatment of osteoarthritis includes reducing pain and/or improving joint movement. Treatment of psoriasis includes the reduction of symptoms of the disease, such as reducing the shedding of skin, or controlling the progression of the disease. Treatment includes, therefore, methods for reducing inflammation associated with psoriasis. As used herein “controlling the progression of the disease” refers to any reduction in the rate of the progression of the disease. The term also includes halting disease progression.

The methods and compositions provided herein, therefore, in some embodiments include treatments used in osteoarthritis or psoriatic subjects. Other osteoarthritic treatments include NSAIDS and corticosteroids. Other psoriatic treatments include steroids, such as cortisone; scalp treatment with coal tar or cortisone (at times in combination with salicylic and lactic acid); anthralin; vitamin D (synthetic vitamin D analogue (calcipotriene)); retinoids (prescription vitamin A-related gels, creams (tazarotene), and oral medications (isotrentinoin, acitretin)); coal tar; Goeckerman Treatment (coal tar dressings and ultraviolet light); light therapy (Ultraviolet light B (UVB)); psoralen and UVA (PUVA); methotrexate; cyclosporine; alefacept; etancercept; infliximab; adalimumab; and efalizumab.

An “infection or infection related disorder” refers to any condition that results from the presence of one or more pathogenic microorganisms in the body of a subject.

An “allergic disorder” is any condition that is the result of the body's improper sensitivity to an allergen. The allergen can be a self or non-self antigen. The term is meant to include allergies and allergic reactions. Allergic disorders include but are not limited to eczema, allergic rhinitis or coryza, hay fever, conjunctivitis, bronchial asthma, urticaria (hives) and food allergies, and other atopic conditions. Agents used to treat allergic disorders are known in the art and include antihistamines as well as corticosteroids.

### Formulation and Delivery

The compositions and methods provided herein can be used, depending on the biological agents selected and the assay used to select them, to treat or prevent a number of disorders. For instance, in some embodiments the compositions and methods provided are useful for preventing and/or treating coagulation, angiogenesis, thrombotic disorders, cardiovascular disease, vascular conditions, cerebrovascular conditions, stroke, atherosclerosis, neurodegenerative disease, macular degeneration, respiratory disorders, asthma, inflammatory disorders, immunologic disorders, lupus, allergic disorders, circulatory shock and related disorders, central nervous system disorders, Alzheimer's disease, dermatological disorders, psoriasis, inflammatory bowel disease, Crohn's disease, ulcerative colitis, fibrotic lungs, infection or an infection related disorder, pseudomonas infection, S. aureus infection, human immunodeficiency virus (HIV) infection, or inhibiting cancer cell growth, reducing tumor size, preventing cancer invasiveness, inhibiting cancer progression, and inhibiting metastasis.

The compositions and treatments provided can also be used to prevent and/or treat disorders which include diabetes, encephalitis, hydrocephalus, obesity, varicose veins, vasculititides, lymphangitis, lymphedema, hypertension, superior vena caval syndrome, myocarditis, restrictive cardiomyopathy, pericarditis, hereditary hemopoetic disorders, disseminated intravascular coagulation, restrictive lung diseases, obstructive lung disease, cystic fibrosis, gastrointestinal ulcerations, Wilson's disease, alpha1-antitrypsin disease, cholecystsitis, gall stones, kidney stones, renal and bladder infections/urinary tract infections or protein deficiencies (e.g. Tay Sachs). The compositions and methods provided can also be used to promote neural regeneration and/or spinal cord repair, reverse or promote hair loss, or reverse or inhibit hearing loss.

Each of these disorders is well-known in the art and/or is described, for instance, in Harrison's Principles of Internal Medicine (McGraw Hill, Inc., New York), which is incorporated by reference.

In one embodiment of the invention, prevention of a disease, disorder, condition or trait in a subject that is associated with aberrant or unwanted target gene expression or activity may be achieved by administering to the subject a therapeutic agent comprising a double-stranded oligoribonucleotide of the invention targeting one or more target nucleic acids. Subjects at risk for a disease, disorder, condition or trait which is caused or contributed to by aberrant or unwanted target gene expression or activity can be identified by diagnostic or prognostic assays as known in the art and as described herein. Application or administration of, or introduction into a subject of, one or more double-stranded oligoribonucleotides of the invention as a prophylactic agent can occur prior to the manifestation of symptoms characteristic of the target gene aberrancy, such that a disease or disorder is prevented or, alternatively, delayed in its progression. In the context of the invention, such double-stranded oligoribonucleotides may be target gene agonists or target gene antagonists depending on the nature of the target gene aberrancy. Whether to select an appropriate agonistic or antagonistic agent can be determined based on screening assays described in the art or as set forth herein.

For better administration, the composition may further contain at least one kind of pharmaceutically acceptable carriers in addition to the above-described active ingredients. It is important that the pharmaceutically acceptable carriers be compatible with the active ingredients of the present invention. Examples of such carriers include saline solution, sterile water, Ringer's solution, buffered saline solution, dextrose solution, maltodextrin (aqueous) solution, glycerol, ethanol and a mixture thereof. If needed, typical additives, such as, an antioxidant, a buffer, a bacteriostatic agent and the like, may be added. Moreover, the composition can be pharmaceutically produced for injection in form of aqueous solution, suspension, emulsion and so forth by adding more additives, such as, a diluting agent, a dispersing agent, a surfactant, a bonding agent and a lubricant. Further, the composition may be prepared for pharmaceutical application depending on the types of disease or the ingredient, by employing conventional methods or the methods described in Remington's Pharmaceutical Science (late edition), Mack Publishing Company, Easton Pa.

The pharmaceutical composition of the present invention can be defined by an expert in the technical field in which the invention applies, based on a typical symptom of a patient and the seriousness of the disease. The composition can be prepared in diverse forms, such as, powder, tablet, capsule, solution, injection, ointment, syrup and the like, and provided to patients in single dose container or multi-dose container, for example, in a sealed ampoule or bottle. The pharmaceutical composition of the invention can be administered orally or parenterally. Even though there is no limit to the administration route of the pharmaceutical composition, the composition may be brought into contact with the body through diverse administration routes, including oral administration, intravenous administration, intramuscular administration, intra-arterial administration, intramedullary administration, intrathecal administration, intracardiac administration, percutaneous administration, hypodermic administration, intraperitoneal administration, enteral administration, sublingual administration, and topical administration.

For such clinical administration, the pharmaceutical composition of the present invention may be prepared in an adequate product using conventional techniques. For instance, if the composition needs to be administered orally, it may be mixed with an inactive diluting agent or an edible carrier, be sealed in hard or soft gelatin capsules, or be pressed into tablets. In case of oral administration, active compounds are mixed with an excipient and are used in form of tablets for intake, buccal tablets, troches, capsules, elixir, suspension, syrup, wafers and the like. On the other hand, in case that the pharmaceutical composition of the present invention is injected or administered parenterally, it can be produced using well-known methods of the technical field in which the invention applies or any conventional methods. Dose of the composition varies depending on a patent's body weight, age, sex, health conditions, diet, timing of administration, administration method, evacuation rate, the seriousness of a disease, etc, and must be determined by an expert (e.g., doctor).

Effective amounts of the compositions of the invention are administered to subjects in need of such treatment. Effective amounts are those amounts which will result in a desired improvement in the condition or symptoms of the condition, e.g., for cancer this is a reduction in cellular proliferation or metastasis, while for neurodegenerative disease or damage this is the regeneration of nerve cells, the prolonged survival of nerve cells, the migration of nerve cells or the restoration of nerve function. Such amounts can be determined with no more than routine experimentation.

It is believed that doses ranging from 1 nanogram/kilogram to 100 milligrams/kilogram, depending upon the mode of administration, will be effective. In some embodiments the level of administration is between 3 micrograms to 14 milligrams per 4 square centimeter area of cells. In one such embodiment it is heparin sodium that is administered at this level in powder or particulate form. The absolute amount will depend upon a variety of factors (including whether the administration is in conjunction with other methods of treatment, the number of doses and individual patient parameters including age, physical condition, size and weight) and can be determined with routine experimentation. It is preferred generally that a maximum dose be used, that is, the highest safe dose according to sound medical judgment. The mode of administration may be any medically acceptable mode including oral, ocular, topical, transdermal, rectal, nasal, subcutaneous, intravenous, etc. or via administration to a mucous membrane. In some embodiments the mode of administration is topical administration. In one embodiment the administration is via the internal carotid artery.

In general, when administered for therapeutic purposes, the formulations of the invention are applied in pharmaceutically acceptable solutions. Such preparations may routinely contain pharmaceutically acceptable concentrations of salt, buffering agents, preservatives, compatible carriers, adjuvants, and optionally other therapeutic ingredients.

The compositions of the invention may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic and benzene sulphonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

The present invention provides pharmaceutical compositions, for medical use, which comprise the polysaccharides provided and/or the polysaccharide-degrading enzymes together with one or more pharmaceutically acceptable carriers and optionally other therapeutic ingredients. The pharmaceutical compositions can also, in some embodiments, include one or more biologically active molecules. The term “pharmaceutically-acceptable carrier” as used herein, and described more fully below, means one or more compatible solid or liquid filler, dilutants or encapsulating substances which are suitable for administration to a human or other animal. In the present invention, the term “carrier” denotes an organic or inorganic ingredient, natural or synthetic, with which the active ingredient is combined to facilitate the application. The components of the pharmaceutical compositions also are capable of being comingled with each other, in a manner such that there is no interaction which would substantially impair the desired pharmaceutical efficiency.

A variety of administration routes are available. The particular mode selected will depend, of course, upon the particular active agent(s) selected, the desired results, the particular condition being treated and the dosage required for therapeutic efficacy. The methods of this invention, generally speaking, may be practiced using any mode of administration that is medically acceptable, meaning any mode that produces effective levels of RNAco-i without causing clinically unacceptable adverse effects. One mode of administration is the parenteral route. The term “parenteral” includes subcutaneous injections, intravenous, intramuscular, intraperitoneal, intrasternal injection or infusion techniques. Other modes of administration include oral, mucosal, rectal, vaginal, sublingual, intranasal, intratracheal, intracranial, inhalation, ocular, topical, transdermal, etc. In some embodiments the administration of the compositions does not occur via the pulmonary route.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, diskus or a spacer. In certain of these embodiments the inhaler is a Spinhaler (Rhone-Poulenc Rorer, West Malling, Kent). Other medical devices are known in the art and include the following technologies Inhale/Pfizer, Pharmaceutical Discovery Corporation/Mannkind/Glaxo and Advanced Inhalation Technologies/Alkermes.

The compounds, when it is desirable to deliver them systemically, may be formulated for parenteral administration by injection, e.g. by bolus injection or continuous infusion. Formulations for injection may be presented in unit dosage form, e.g. in ampoules or in multi-dose containers, with an added preservative. In some embodiments the compounds provided are administered by infusion pump. In some of these embodiments the compounds are administered by infusion pump to be delivered to the blood brain barrier. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990 and Langer and Tirrell, Nature, 2004 Apr. 1; 428(6982): 487-92, which are incorporated herein by reference.

The compositions may conveniently be presented in unit dosage form and may be prepared by any of the methods well known in the art of pharmacy.

In some embodiments the composition that is administered is in powder or particulate form rather than as a solution. In some embodiments the compositions that is administered includes sodium heparin in powder or particulate form. Examples of particulate forms contemplated as part of the invention in some embodiments are provided in U.S. patent application Ser. No. 09/982,548, filed Oct. 18, 2001, which is hereby incorporated by reference in its entirety. In other embodiments the compositions are administered in aerosol form. In other embodiments the method of administration includes the use of a bandage, slow release patch, engineered or biodegradable scaffold, slow release polymer, tablet or capsule.

In other embodiments the RNAco-i agent, depending on the RNAco-i agent, is administered via a route that is not normally associated with administering the RNAco-i agent for therapeutic purposes. In some embodiments the RNAco-i agent is not administered via a pulmonary route. In other embodiments the RNAco-i agent is not administered via a gastrointestinal and/or oral route. In still other embodiments the RNAco-i agent is not administered intravenously and/or subcutaneously. In yet other embodiments the RNAco-i agent is not administered topically. In still other embodiments, the RNAco-i agent is not administered transdermally.

Other delivery systems can include time-release, delayed release or sustained release delivery systems. Such systems can avoid repeated administrations of the compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775 (Kent); 4,667,014 (Nestor et al.); and 4,748,034 and 5,239,660 (Leonard) and (b) diffusional systems in which an active component permeates at a controlled rate through a polymer, found in U.S. Pat. Nos. 3,832,253 (Higuchi et al.) and 3,854,480 (Zaffaroni). In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of preferred non-biodegradable polymers include ethylene vinyl acetate, poly(meth) acrylic acid, polyamides, copolymers and mixtures thereof.

Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

In another embodiment slow release is accomplished with the use of polyanhydride wafers.

The compositions can be administered locally or the compositions can further include a targeting molecule. The targeting molecule can be attached to the RNAco-i vehicle preferably through the non-active carrier portion, either as the compound between the various active agents or as a component of the formulation. A targeting molecule is any molecule or compound which is specific for a particular cell or tissue and which can be used to direct the agents provided herein to a particular cell or tissue. Targeting molecules can be any molecule that is differentially present on a particular cell or in a particular tissue. These molecules can be proteins expressed on the cell surface. In one embodiment the targeting molecule targets a particular cell barrier. The cells/cell barrier can be any cells/cell barrier as provided herein. The targeting molecules can be any molecule that preferentially targets a particular molecule associated with a particular cell/cell barrier. In one embodiment the cell barrier is the blood brain barrier. In another embodiment the targeting molecule is an antibody (e.g., a monoclonal antibody (mAb) to a receptor present on the blood brain barrier). In one embodiment the targeting molecule is an antibody, such as monoclonal antibody OX26, to transferrin receptor (present in the blood brain barrier as well as the liver in higher amounts than in other tissues). In another embodiment the targeting molecule is a monoclonal antibody to PGP1 (P-glycoprotein 1). In another embodiment the targeting molecule is a monoclonal antibody to EGFR.

Targeting molecules can in some embodiments be used to target disease markers. In one embodiment the targeting molecule is a molecule which specifically interacts with a cancer cell or a tumor. For instance, the targeting molecule may be a protein (e.g., an antibody) or other type of molecule that recognizes and specifically interacts with a tumor antigen.

Tumor-antigens include Melan-A/MART-1, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmel117}, PRAME, NY-ESO-1, brain glycogen phosphorylase, SSX-1, SSX-2 (HOM-MEL-40), SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, Smad family of tumor antigens, Imp-1, EBV-encoded nuclear antigen (EBNA)-1, and c-erbB-2.

Some aspects of the invention also encompass kits. The kits of the invention include one or more RNAco-i agents. The kits can further include one or more biologically active molecules, administration devices (e.g., an inhalation apparatus) and/or instructions for use. An inhalation apparatus, as used herein, is any device for administering a dry aerosol. This type of equipment is well known in the art and has been described in detail, such as that description found in Remington: The Science and Practice of Pharmacy, 19^{th} Edition, 1995, Mac Publishing Company, Easton, Pa., pages 1676-1692. Many U.S. patents also describe inhalation devices, such as U.S. Pat. No. 6,116,237. The kits provided can also include an RNAco-i inhibiting agent and/or a detection system. Detection systems can be used to determine the amount of any or all of the agents administered in the blood. Detection systems can be invasive or non-invasive. An example of an invasive detection system is one which involves the removal of a blood sample and can further involve an assay such as an enzymatic assay or a binding assay to detect levels in the blood. A non-invasive type of detection system is one which can detect the levels of the agent in the blood without having to break the skin barrier. These types of non-invasive systems include, for instance, a monitor which can be placed on the surface of the skin, e.g., in the form of a ring or patch, and which can detect the level of circulating agents. One method for detection may be based on the presence of fluorescence in the agent which is administered. Thus, if a fluorescently labeled agent is administered and the detection system is non-invasive, it can be a system which detects fluorescence. This is particularly useful in the situation when the patient is self-administering and needs to know the blood concentration or an estimate thereof in order to avoid side effects or to determine when another dose is required.

A subject is any human or non-human vertebrate, e.g., dog, cat, horse, cow, monkey, pig, mouse, rat.

The invention, in a preferred embodiment, is useful for treating tumor cell proliferation or metastasis in a subject. The terms “treat” and “treating” as used herein refer to inhibiting completely or partially the proliferation or metastasis of a cancer or tumor cell, as well as inhibiting any increase in the proliferation or metastasis of a cancer or tumor cell. Treat or treating also refers to retarding the proliferation or metastasis of tumor cells in a subject. Additionally, treat or treating may include the elimination or reduction of the symptoms associated with the tumor cell proliferation or metastasis.

A “subject having a cancer” is a subject that has detectable cancerous cells. The cancer may be a malignant or non-malignant cancer. A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos or other chemical toxins, or a subject who has previously been treated for cancer and is in apparent remission. When a subject at risk of developing a cancer is treated with the compositions provided the subject may be able to kill the cancer cells as they develop.

The compositions may also be used, for instance, in a method for inhibiting angiogenesis. In this method an effective amount for inhibiting angiogenesis of the composition is administered to a subject in need of treatment thereof. Angiogenesis as used herein is the inappropriate formation of new blood vessels. “Angiogenesis” often occurs in tumors when endothelial cells secrete a group of growth factors that are mitogenic for endothelium causing the elongation and proliferation of endothelial cells which results in a generation of new blood vessels.

In some aspects of the invention the effective amount of the compositions is that amount effective to prevent invasion of a tumor cell across a barrier. The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers. Thus the term “metastasis” as used herein refers to the invasion and migration of tumor cells away from the primary tumor site.

The barrier for the tumor cells may be an artificial barrier in vitro or a natural barrier in vivo. In vitro barriers include but are not limited to extracellular matrix coated membranes, such as Matrigel. Thus the compositions can be tested for their ability to inhibit tumor cell invasion in a Matrigel invasion assay system as described in detail by Parish, C. R., et al., “A Basement-Membrane Permeability Assay which Correlates with the Metastatic Potential of Tumour Cells,” Int. J. Cancer (1992) 52:378-383. Matrigel is a reconstituted basement membrane containing type IV collagen, laminin, heparan sulfate proteoglycans such as perlecan, which bind to and localize bFGF, vitronectin as well as transforming growth factor-β (TGF-β), urokinase-type plasminogen activator (uPA), tissue plasminogen activator (tPA), and the serpin known as plasminogen activator inhibitor type 1 (PAI-1). Other in vitro and in vivo assays for metastasis have been described in the prior art, see, e.g., U.S. Pat. No. 5,935,850, issued on Aug. 10, 1999, which is incorporated by reference. An in vivo barrier refers to a cellular barrier present in the body of a subject.

It is further provided herein that active agent uptake induced apoptosis is preferential to specific cell types based on internalization rates. Cancer cells, which have a faster endocytic rate than non-cancerous cells, and correspondingly take up polymer-active agent conjugate faster, are typically more susceptible to the effects of the conjugates. While targeting cancer based on endocytic rate alone would likely affect macrophages and neutrophils as well, local delivery could allow for induction of cancer cell death with minimal effects to surrounding tissues. Intratumoral administration can also be used.

Certain cells, such as cancer cells, can also be targeted with the use of a targeting molecule. The compositions provided herein, therefore, can further contain a targeting molecule. The targeting molecule can be physically linked to a active agent or a cationic polymer by any of the methods known in the art. A targeting molecule is any molecule or compound which is specific for a particular cell or tissue and which can be used to direct a active agent; liposome, microsphere or nanoparticle containing the active agent; or a conjugate of the active agent with a cationic polymer to the cell or tissue. The targeting molecule can be directed to any of a number of cells to which the administration of the active agent would be beneficial. The targeted cells therefore include non-immunological cells or non-macrophage cells. The targeted cell may also be non-smooth muscle cells. Targeted cells can also be hyperplastic cells. In some embodiments the targeted cells are cells that internalize the active agent or active agent-cationic polymer conjugate within less than 48 hours. In other embodiments the cells internalize the active agent or active agent-cationic polymer conjugate within less than 24 hours. In another embodiment the cells internalize the active agent or active agent-cationic polymer conjugate within less than 12, 10, 8, 6, 4, 2 or fewer hours. Preferably the cells that are targeted have high endocytic rates, such as cancer cells like epithelial cancer cells. The targeting molecule, therefore, can be a molecule which specifically interacts with a cancer cell or a tumor. For instance, the targeting molecule may be a protein or other type of molecule that recognizes and specifically interacts with a tumor antigen. Targeting molecules, therefore, include antibodies or fragments thereof.

Formulations can also be produced that allow for the temporal release of multiple agents. The agents can be release at different times from the RNAco-i itself through cleavable or degradable moieties. The formulation can additionally allow for differential temporal release of multiple compounds (i.e. RNAco-is). One such means of differential temporal release is through a nanocore wherein a pharmaceutical agent is encapsulated in a lipid vesicle, matrix, or shell containing another such pharmaceutical agent, which forms a nanocell (WO 0814478; Sengupta et al Nature. 2005 Jul. 28; 436(7050):568-72). The agent in the outer portion is a released first followed by the inner portion when the dissolution and/or degradation of the nanocore. Such particles can be of any size commonly producible in the art, typically ranging from 10 nm to 500 μm.

Other formulation-based means of targeting are also employed, including, but not limited to self-assembled biointegrated block copolymers with attached targeting moieties such as aptamers, antibodies, and/or ligands; dendrimers enabling increase avidity of targeting moieties such as aptamers, antibodies, and/or ligands; or capsules requiring focal dissolution or enzymatic activity. Any other formulation-based targeting moiety can additionally readily be used by any person skilled in the art.

Of particular note, the RNAco-i vehicle, independent of the formulation, has intrinsic targetable capacity as the expression of particularly multicipities of targets is likely to be found in particular subsets of cells, most notably those associated with a given disease or disorder. Although other cells may have an overlapping expression pattern, it is likely, in any given cases, that all cells in the body do not have such an expression pattern, with the likelihood of non-target cells having the pattern decreasing with an increased number of active agents, and thereby intrinsic targeting is achieved.

### Target and Active Agent Selection

Multiple approaches have been described in the art to define the various targets and associated active agents that can have valuable impacts on a biological system. In its simplest form, empirical methods have been used. Herein, two or more drugs are combined over a combinatorial dose array with phenotypic measurements made on an assay system that has been defined to be relevant. These systems are most effective in high throughput. Furthermore, these systems can be rapidly adaptable to any drugs that can work in any existing system. The data derived from these systems is also representative of actual biology although the effect may be limited to the assay system employed. Fundamentally, however, such combination identification systems are limited to targets and moieties for which chemical agents (protein, small molecule, etc) already exist. As RNAi can be designed to target any genetic moiety as a function of sequence (though efficacy requires validity), in silico methods can be rapidly employed to define targets to be inhibited, which can then be achieved preferentially by RNAi, but also by other molecular inhibitors.

In the instant invention, multiple in silico screening approaches are used to define the targets and the corresponding activity of active moieties to be used in combination. The various methods described are readily adaptable to define combinations of two or more targets and/or agents when the appropriate mathematical filters are used to define optimal combinations based on a selected output. Three preferred methods are described herein. These include the Institute for Systems Biology measurement approach, the Genstruct causal modeling approach, and the Collins mathematical approach, and the Entelos mathematical modeling approach. It should be obvious to one of skill in the art that other such approaches not explicitly described herein can also be readily applied in the selection of combinations.

Certain aspects of the present invention relate to identification of two or more gene targets whose inhibition by a first agent and a second agent elicit synergistic therapeutic effect. In some embodiments, the identification (or discovery) process is rational. In these embodiments, the rationale can include explicit a priori knowledge of the disease or condition biology. By way of non-limiting example, the first and second agent may be selected to inhibit a first and second gene target each independently implicated by published literature to influence the etiology, maintenance, or progression of the disease. In other embodiments, the rationale can include hypotheses based on computational or systems biology. Said hypotheses can be generated based on quantitative or qualitative differences in the levels of one or more biomolecules (e.g. mRNA, DNA, miRNA, protein or lipid) between a healthy and diseased cell, tissue, or organism. In some embodiments, the first agent and the second agent target unique “nodes” within a computationally-derived biological network (e.g. a causal network model, a reverse-engineered network model, a Bayesian orthogonal least squares network model, etc.) [Pollard J et al. “A computational model to define the molecular causes of Type 2 diabetes mellitus.” Diabetes Tech & Ther (2005), 7(2):323-336; Ergun A et al. “A network biology approach to prostate cancer.” Mol Sys Bio (2007); 3:82; Kim CS. “Bayesian orthogonal least squares (BOLS) algorithm for reverse engineering of gene regulatory networks.” BMC Bioinformatics (2007); 8:251].

In some embodiments, the second agent is selected to inhibit one or more genes whose expression interferes with the efficacy, potency or safety of the first agent.

In other embodiments, the identification (or discovery process) is empirical. In these embodiments, the first agent and the second agent are combined without explicit a priori knowledge that their introduction (individually or in combination) to a cell, tissue, or organism will influence a disease or condition.

### Institute for Systems Biology Measurement Approach

The general methodology employed by ISB is the collection and analysis of large amounts of quantitative data, focusing on the transcriptome and the proteome. The approaches developed revolve around software, tools, and database frameworks, which are freely available to the public. While multiple such measurement-based approaches have been used, one example involves the acquisition, measurement, and analysis of millions of datapoints generated by comparing an androgen independent (CL1) and androgen-dependent (LNCaP) prostate cancer cell line [Lin B et al. “Evidence for the presence of disease-perturbed networks in prostate cancer cells by genomic and proeteomic analyses: a systems approach to disease.” Cancer Res (2005); 65(8):3081-3091]. The first source of data utilized massively parallel signature sequencing (MPSS), an approach in which 20 nucleotide signature sequences are sequenced in parallel for more than 1 million DNA sequences derived from a cDNA library. The raw data is represented in terms of transcripts per million (tpm), an approach which is sensitive enough to allow for quantitation of even low abundance transcripts. In contrast, standard microarray or differential display technologies typically exhibit limited sensitivity and dynamic range and therefore provide limited or no data on low abundance transcripts. In addition to a deep quantitative analysis of RNA abundance via MPSS, this study utilized isotope-coded affinity tags (ICAT) coupled with tandem mass spectrometry (MS/MS) to generate quantitative peptide ratios for 4,583 peptides corresponding to 940 distinct proteins. By integrating deep RNA and protein data, 37 BioCarta and 14 KEGG pathways were found to be upregulated in LNCaP versus CL1 cells. In addition, 23 BioCarta and 22 KEGG pathways were downregulated in LNCaP versus CL1 cells. By capturing and distilling millions of datapoints into a small and manageable number of pathways, this Systems Biology approach generates a tractable set of hypotheses that can be tested experimentally. In particular, pathways upregulated at both the mRNA and protein levels in the more aggressive androgen-independent cell line may be good targets to downregulate with two or more siRNAs.

Other such methods have been detailed in Gilchrist M et al. Systems Biology Approaches Identify ATF3 as a Negative Regulator of Innate Immunity. Nature (2006). 441:173-8; Yi E C et al. Increased quantitative proteome coverage with (13)C/(12)C-based, acid-cleavable isotope-coded affinity tag reagent and modified data acquisition scheme. Proteomics. 2005 February; 5(2):380-7; Lin B et al. “Evidence for the presence of disease-perturbed networks in prostate cancer cells by genomic and proeteomic analyses: a systems approach to disease.” Cancer Res (2005); 65(8):3081-3091; Smith J J et al. Transcriptome profiling to identify genes involved in peroxisome assembly and function. J Cell Biol. 2002 Jul. 22; 158(2):259-71; Marelli M et al. Quantitative mass spectrometry reveals a role for the GTPase Rho1p in actin organization on the peroxisome membrane. J Cell Biol. 2004 Dec. 20; 167(6):1099-112; Reiss D J et al. Integrated biclustering of heterogeneous genome-wide datasets for the inference of global regulatory networks. BMC Bioinformatics. 2006 Jun. 2; 7(1):280; Bonneau R et al. The Inferelator: an algorithm for learning parsimonious regulatory networks from systems-biology data sets de novo. Genome Biol. 2006 May 10; 7(5):R36; Shannon P et al. Gaggle: An open-source software system for integrating bioinformatics software and data sources. BMC Bioinformatics, 2006 Mar. 28; 7(1):176; Yan W et al. System-based proteomic analysis of the interferon response in human liver cells. Genome Biol. 2004; 5(8):R54. Epub 2004 Jul. 22; US 20060009915 Rapid and quantitative proteome analysis and related methods; WO 05114221 Compositions and methods for quantification of serum glycoproteins; WO 02007677 Affinity capture of peptides by microarray and related methods; WO 04058051 Androgen-regulated genes and uses for diagnosis, prognosis, and treatment of prostate neoplastic conditions; WO 04019000 Chemical reagents and methods for detection and quantifications of proteins in complex mixtures; WO 03102220 Methods for high throughput and quantitative proteome analysis; WO 03102018 Methods for quantitative proteome analysis of glycoproteins; WO 03065034 Gene discovery for the system assignment of genes function; WO 03060148 Androgen regulated nucleic acid molecules and encoded proteins; WO 03003810 Methods for detection and quantification of analytes in complex mixtures; WO 02093131 Methods for isolating and labeling sample molecules; WO 02085933 Toll-like receptor 5 ligands and methods of use; WO 02083923 Methods for quantification and de novo polypeptide sequencing by mass spec; WO 02052259 Rapid and quantitative proteome analysis and related methods; WO 02046410 Prostate-specific polypeptide PAMP and encoding nucleic acid molecules; WO 02010456 Multiparameter analysis for predictive medicine. These references describe in detail the methods by which one whom is skilled in the art can use and apply the Institute for Systems Biology measurement approach, and are hereby incorporated by reference. The various methods described in detail in these manuscripts are readily used to define combinations of targets that can have desirable effects when analyzed through an appropriate mathematical filter (as follows).

### Genstruct Causal Modeling

The Genstruct method involves digitizing biological knowledge into a defined set of relationships. This method is depended on a large central knowledge base from manual and automated extraction of biological knowledge from various publicly available databases including Locuslink, GO, OMIM, CSNDB, KEGG, and Homologene. For specific inquiries, the relevant portions of the model are extracted. Data are transformed into simple computable cause and effect relationships, and artificial intelligence is used to reason through the relationships to generate millions of potential hypotheses, which are then individually scored and ranked to produce a coherent set of experimentally testable scientific hypotheses. The causal model is the product of frame-based knowledge representation (Minskey M. “Logical versus analogical or symbolic versus connectionist or neat versus scruffy.” A I Mag (1991); 12(2):34-51) and “Lego block-like” templates to represent common biological functions such as activation, transcription, phosphorylation, binding, and transport (see Elliston K O. “Breaking through the cognitive barriers that impede critical path research.” Am Biotech Lab (2004); 22(12): 26-30). The resultant method has been well described in Elliston KO. “Breaking through the cognitive barriers that impede critical path research.” Am Biotech Lab (2004); 22(12): 26-30; Kightley D A, Chandra N, and Elliston K. “Inferring gene regulatory networks from raw data—a molecular epistemics approach.” Pacific Symposium on Biocomputing, World Scientific Press (2004); Pollard J et al. “A computational model to define the molecular causes of Type 2 diabetes mellitus.” Diabetes Tech & Ther (2005); 7(2):323-336; Lieu C A and Elliston KO. “Applying a Causal Framerwork to System Modeling” pgs 140-152 in Systems Biology: Applications and Perspectives, (ed) Bringmann P et al. Springer (2006); United States Patent Application 20040249620 Epistemic Engine; United States Patent Application 20050165594 System, method and apparatus for causal implication analysis in biological networks; United States Patent Application 20050038608 System, method, and apparatus for assembling and mining life science data; United States Patent Application 20050154535 Method, system and apparatus for Assembling and using biological knowledge; United States Patent Application 20060140860 Computational knowledge model to discovery molecular causes and treatments of diabetes mellitus; United States Patent Application 20070225956 Causal analysis in complex biological systems. These references describe in detail the methods by which one whom is skilled in the art can use and apply the Genstruct Causal Modeling approach, and are hereby incorporated by reference.

This method has been readily applied to define key “on target” drugs, which can be readily adapted to use multiple simultaneous inputs. In a particular instance, cooperative target identification can be performed through reversal causal modeling by identifying causal genes in the disease versus normal data sets. In another example, forward causal modeling can be directly applied to define the specific direction of one or more targets. Dose and/or RNAi efficaciousness can be similarly modeled through percentage of downregulation. This methodology is readily adaptable to multiple cell types, tissues, organisms, etc. Reverse causal modeling can additionally be employed as described to define off-target effects that a second agent could mitigate, as well as resistance mechanisms that a second agent could bypass or reverse.

### Collins Mathematical Modeling

The Collins mathematical approach, “mode-of-action by network identification” (MNI) involves a network model of regulatory interactions is reverse engineered with a diverse training set of whole-genome expression profiles which is used as a filter to determine the genes affected by a condition of interest, for example a disease (Ergun A et al. “A network biology approach to prostate cancer.” Mol Sys Bio (2007); 3:82). In this approach, a genetic “network” is extracted from large (publicly available or private) expression profiling data and expressed as a set of differential equations or difference equations in which the activities of each of the individual elements of the network are represented by variables. The specific application of this approach has been well described in Di Bernardo D et al. “Robust identification of large genetic networks.” Pac Symp Biocompu (2004); 486-97; Du Bernardi D et al. “Chemogenomic profiling on a genome-wide scale using reverse engineered gene networks.” Nat Biotech (2005); 23:377-83; Ergun A et al. “A network biology approach to prostate cancer.” Mol Syst Biol (2007); 3:82; Faith J J et al. “Large-scale mapping and validation of Escherichia coli transcriptional regulation from a compendium of expression profiles.” PLOS Biol (2007); 5(1):e8; United States Patent Application 20060293873 Systems and methods for reverse engineering models of biological networks (“MNI#1”); and United States Patent Application 20070016390 Systems and methods for reverse engineering models of biological networks (“MNI#2”). These approaches are readily adaptable to one skilled in the art to identify candidate cooperative targets. These references also describe in detail the methods by which one whom is skilled in the art can use and apply the Collins Mathematical Modeling approach, and are hereby incorporated by reference. Specifically, the MNI algorithm can readily identify candidate targets based on predicated disease mediators. Resistance pathways can additionally be predicted. This approach is optimally used to identify particular targets rather than to mathematically define the specific contributions of particular combinations. When combined with other approaches, or empirical data, this approach serves as a key target identification approach.

### Entelos Mathematical Modeling

The Entelos PhysioLab platform employs a top-down approach that synthesizes quantitative data from thousands of peer-reviewed papers into a single contextual framework, thereby providing an understanding of human physiology in both health and disease. The models are used to simulate novel therapeutic strategies, new experimental approaches and clinical trials, all with the aim of predicting downstream human efficacy. The PhysioLab is a mechanistic mathematical model that describes human physiology with a set of differential equations. These approaches are described in U.S. Pat. No. 7,165,017 Method and apparatus for conducting linked simulation operations utilizing a computer-based system model; U.S. Pat. No. 6,983,237 Method and apparatus for conducting linked simulation operations utilizing a computer-based system model; U.S. Pat. No. 6,539,347 Method of generating a display for dynamic simulation model utilizing node and link representations; U.S. Pat. No. 6,078,739 Method of managing objects and parameter values associated within a simulation model; WO 06084196 Method for defining virtual patient populations; WO 05036446 Simulating patient-specific outcomes; WO 05026911 Apparatus and method for identifying therapeutic targets using a computer model; WO 04114195 Predictive toxicology for biological systems. These references also describe in detail the methods by which one whom is skilled in the art can use and apply the Entelos Mathematical Modeling approach, and are hereby incorporated by reference. This platform is readily adaptable to applying multiple putative therapeutic agents (i.e. multiple RNAi conferring moieties) and, in combinatorial fashion, predicting, with the appropriate mathematical filter, defining preferred combinations.

### Mathematical Filter on Target Selection

Optimal combinations are those wherein the administration of two active moieties has an effect that cannot be achieved with just one. Intuitively, a combination of agents can yield an effect greater than, less than, or equivalent to the mathematic summation of the effects of (appropriate given the assay at hand) individual agents. The network analysis approaches are used to identify node that represent therapeutic opportunities. In generating combinations, the network analysis is run looking for desirable as well optimal effects through the identical output mode. In one embodiment combinations of two or more active moieties are selected that achieve the maximal effect possible. Such an effect is defined through an in silico analysis, exhaustive or based on design of experiments that produces the maximal effect based on the defined measurement or phenotype. In another embodiment, combinations of two or more active moieties are selected that achieve an increased effect relative to other approaches to achieve a given phenotype. In this embodiment, the combination of the first and second agent can be additive, as in certain cases, even an additive response can be sufficient to provide for an improved therapeutic.

In a preferred embodiment, combinations of two or more active moieties are selected that achieve a synergistic response. Synergy is defined as the interaction of two or more agents or forces so that their combined effect is greater than the appropriate mathematic summation of their individual parts (The American Heritage Dictionary). Implicit in this definition is a non-obvious benefit that would not have been predicted by simply viewing the responses seen with the individual contributors. Additionally, synergistic combinations achieve (or exceed) a specified effect (e.g. a therapeutic effect) at a lower total dose than required when the agents are administered individually. A lower dose provides commercial advantages, since a lower amount of active ingredients reduces often translates into a reduction in the overall cost of manufacturing. In addition, the lower total dose may provide secondary benefits, for example a reduction in off-target or side-effects.

While an interaction or network analysis can define or predict what elements are synergistic, an analytic step beyond simply measuring readouts is necessary to define. In this instant invention, predictive models as described are used with methods taught herein and in other cases that can develop an understanding of synergistic cases. Given that synergy is by definition, non-obvious, all synergistic combinations are therefore validated empirically even after identification through various screens.

Synergy is defined in one of several ways based on the specific case. The scenarios comprise 1) independent similar action, 2) the framework when one or more (not all) agents lacks efficacy alone for the specific effect, and 3) a generalizable case independent of the efficacy of agents.

### Independent Similar Action

In this scenario, two agents produce a common effect through mechanisms not related to a common receptor or target, which is defined as “similar and independent” (Bliss C I. “The toxicity of poisons applied jointly.” Ann Appl Biol (1939); 26:585-615).

One method commonly used in the art to assess whether a combination of two agents is additive is the isobologram. In this method, introduced by Loewe in the 1950s (Loewe S. “The problem of synergism and antagonism of combined drugs.” Arzneimittelforschung (1953); 3:285-90, and Loewe S. “Antagonism and antagonists.” Pharmacol Rev (1957); 9:237-242), a graph is constructed displaying equally effective dose pairs (“isoboles”) for a single effect level. A specific effect is first selected, for example a 50% reduction in cell number. Next, doses of drug A and drug B (each alone) that give this effect are plotted as axial points in a Cartestian X/Y plot. Next, a straight line is drawn connecting the axial points A and B, which define the multiple points or “dose pairs” that will produce the selected effect in a simply “additive” fashion. Subsequently, the actual experimentally determined dose pair producing this specified effect is plotted. If the dose pair falls below the “line of additivity,” the combination is superadditive or synergistic. In contrast, if the dose pair lies above the line of additivity, the combination is subadditive (or antagonistic). Dose pairs falling on or near the line of additivity are deemed merely additive.

To gain statistical clarity, a regression analysis is performed on a subset of data points that appear below the line of additivity (e.g. are presumed to be synergistic). This approach has been previously described extensively with mathematical details [Tallarida R J. “Statistical analysis of drug combinations for synergism.” Pain (1992); 49(1):93-7; and Tallarida R J. “Drug synergism and dose-effect data analysis.” (2000) Chapman Hall/CRC Press, Boca Raton]. In brief, the total dose (Zt) for the specified effect is plotted against the fraction (fA) of drug A's potency (A) in each combination. For a particular EA, the total dose for the specified effect, along with its variance, is obtained from a standard regression analysis of the data. The Zₜ and the total additive dose [defined as f_{A}A+(1−f_{A})B] can be tested for a significant difference using the Student t distribution test. Weighted regression procedures are critical when examining quantal dose-effect data. Simple linear regression is often sufficient, but when nonlinear curve fitting is preferred or required, a variety of commercially available standard software packages can be applied (e.g. MAT-LAB, Mathworks, Natick, Mass. or PharmToolsPro, The McCary Group, Elkins Park, Pa.).

The classic isobologram employs sets of equally effective dose combinations and is therefore limited in application to the single, specified effect (e.g. 50% reduction in cell number). Tallarida (1997, 2000, 2001) has described a generalized version of the isobolar analysis to examine drug combinations over the range of effects (e.g. 30, 40, 50, 60, 70, 80% reduction in cell number), which is particularly useful if the relative potency of the two agents vary. In this approach, the individual dose-response curves for drug A and drug B are used to construct a curve for the fixed-ratio combination in which the proportion of the total dose that is drug A is defined as p_{A} and the proportion of the total dose that is drug B is defined as p_{B}, or 1−p_{A}. This approach thus uses the relative potency values over the range of the effects common to drugs A and B, and is therefore known as the Additive Composite Curve. Experimental data with fixed proportion formulations (actual total-dose effect) can be statistically compared to the composite additive curve using an analysis of variance (ANOVA) procedure on the log dose-effect data. As an extension of the Additive Composite Curve, the combined actions can be represented using a three-dimensional Response Surface Analysis in which the doses are plotted as Cartesian coordinates in the x-y planes, and the effect is plotted as the vertical distance above this planar point (Tallarida 1999, 2000, 2001; Kong M and Lee J J. “A generalized response surface model with varying relative potency for assessing drug interaction.” Biometrics (2006); 62(4):986-995). The compendium of combination dose-effect points can be fitted using curve-fitting approaches yielding a smooth surface representing the additivity of the combination (analogous to the “line of additivity” in the standard isobologram). Experiments leveraging this analysis technique (e.g. Tallerida 1999) underscore that synergy is not merely a property of the drug combination, but also depends on the ratio of the compounds and the endpoint used.

An alternative framework has been described by Chou and Tallalay (Chou T and Talalay P. “A simple generalized equation for the analysis of multiple inhibitions of Michaelis-Menten kinetic systems.” J. Biol Chem (1977); 252(18):6438-42). This framework is applicable to characterizing the combination effects of multiple exclusive inhibitors targeting a single enzymatic reaction. In this model, a combination is synergistic when it is determined that the enzymatic velocity in the presence of both agents (v_{1,2}) satisfies 1/(v_{1,2})>1/v₁+1/v₂−1/v₀. In contrast, a combination is antagonistic if v_{1,2}<1/v₁+1/v₂−1/v0.

Framework when One or More Agents Lacks Efficacy for the Specific Effect

The above mathematical approaches breaks down in scenarios in which at least one the agents alone has no efficacy for the specific effect. Gorny and colleagues have described and validated a framework that effectively and accurately characterizes the combinatorial effects multiple active moieties when one of the agents has no detectable response based on the selected readout when applied alone (Verrier F et al. “Additive effects characterize the interaction of antibodies involved in neutralization of the primary dualtropic HIV-1 isolate 89.6.” J Virol (2001); 75(19):9177-86). In this method, the combination effect of two agents is based on the comparison of the experimental effect of the combination of the two agents to the effect predicted under the hypothesis that the two agents act neither in synergy nor antagonism but rather in a statistically independent manner. As specifically described in the case of antibodies neutralizing HIV-1 from the original publication, p1 is defined as the probability at dose (concentration) D1 that the first agent neutralizes HIV-1, which is estimated by the proportion (fraction) of cells protected from the HIV-1 infection by the single agent at D1. Similarly, p₂ is probability that agent 2 at dose D2 neutralizes HIV-1. Based on p₁ and p₂ and an assumption of statistical independence, the probability that the combination (p₁₂) will neutralize HIV-1 is defined as (p₁₂)=p₁+p₂−p₁p₂. Gorny and colleagues further define E_{ind} as the probability that a specific virus particle is neutralized by at least one of the two antibodies, whereby E_{ind}=1−q₁q₂ and q1 is the probability that the first antibody alone fails to neutralize and q2 is the probability that the second antibody alone fails to neutralize the particle. Of note, E_{ind}=p₁+p₂−p₁p₂=1−q₁q₂. Using this paradigm, experimental results E (typically represented by E*, which is an average of some number of replicates of E) is compared to E_{ind}. Synergy is defined as combinations at a fixed concentration D such that E>E_{ind}. Antagonism is defined as combinations at a fixed concentration D such that E<E_{ind}. Statistical rigor can be applied by averaging experimental results from multiple replicates (E*) and similarly [though in a separate experiment] averaging multiple replicates of the agents alone to obtain E_{ind}*. Next, the standard deviation (S) can be determined from the relation S²=S²(E*)+S²(Eind*). Thus synergy or antagonism is observed only if |E*−E_{ind}*|/S exceeds a chosen fractile of a Student t test distribution. This method can be readily generalized to any given assay with any given output so long as one of the active moieties does not produce a detectable response in the assay at hand. Of particular note, given the implicit restriction of one active moiety not producing a response, this approach does not require a dose-response curve to be generated for any of the individual agents.

It is also obvious to one of skill in the art that this method can be readily extended to more than two elements added together. Accordingly, the Gorny framework equally applies to combinations of any number of agents (n=2, 3, 4, 5, 6, 7, 8, 9, 10 or more) provided mutual independence among the agents. This assumptions breaks only if two agents compete for a common target (i.e. the same binding site on an enzyme). In the case of RNAi, this assumption does still hold when the multiple active moieties target the same gene in the case that the target sites are non-overlapping. In some instances, even nucleic acid inhibitors targeting overlapping sites on the same gene may be considered mutually independent, provided the number of target molecules (e.g. mRNAs) exceeds the number of nucleic acid inhibitors functionally delivered to the intracellular compartment of the target cell. More importantly, the framework is also applicable when both (when n=2) or all (n>2) of the agents lack efficacy for the specified effect. In this case, E_{ind}*=0 and any combination in which E is non-zero can be classified as synergistic.

In one embodiment, when the Gorny framework, as described, is used when at least one or more active moieties does not have a detectable effect based on the chosen means for a readout. In another embodiment, the Gorny framework, as described, is used when all active moieties lack a detectable effect based on the chosen means for a readout.

In preferred embodiments, the therapeutic effect achieved in combination is never achieved when the first agent or second agent is delivered individually regardless of dose. In these embodiments, the Gorny framework is applicable and synergy is defined because E_{ind}=00 and E>0. In these embodiments, the observed effect is intrinsically non-obvious.

### Generalizable Case Independent of the Individual Efficacy of the Agents

In an alternative and generalizable case, active moieties, typically have phenotypic impacts following a sigmoid curve, which in general, can be characterized by the equation:

P(t)=1/((ê−t)+1) EQ1.

where P(t) represents the signal at concentration t

In this invention, where treatments are looking to reduce signal, the reduction in signal can be correspondingly be represented as

K(t)=1−(1/((ê−t)+1)) EQ2.

where K(t) represents the reduction in signal at concentration t or

K(t)=(ê−t)/((ê−t)+1) EQ3.

The expected additive response from two active moieties when measured based on the ability to reduce a signal is represented by:

K_{T}(t)=K₁(t)*K₂(t) EQ4.

Which is readily generalizable to n signals by:

K_{T}(t)=K₁(t)*K₂(t)*K₃(t)* . . . *Kₙ(t) EQ5.

Synergy is thus defined by the general equation

K_{T}(t)<K₁(t)*K₂(t)*K₃(t)* . . . *Kₙ(t) EQ6.

When applying the above definition of synergy to the case of two active moieties, using the reduction in signal as a measure of interest, the following equation can therefore be applied:

K_{T}(t)<[(ê−t₁)/((ê−t₁)+1)]*[(ê−t₂)/((ê−t₂)+1)] or EQ7.

K_{T}(t)<(ê(t₁+t₂))/(1+(ê−t₁)+(ê−t₂)+ê(−t₁−t₂)) EQ8.

In EQ 7 and 8, the cases where the equation holds define concentrations of agent 1 and agent 2 where a syngergistic reduction in signal is achieved. This notion can be similar extended and generalized to n compounds through the same logic. In the case where n=3, synergy is correspondingly defined by the equation:

K_{T}(t)<(ê−(t₁+t₂+t₃))/(1+(ê−t₁)+(ê−t₂)+(ê−t₃)+ê(−t₁−t₂))+ê(−t₁−t₃))+ê(−t₂−t₃))+ê(−t₁−t₂−t₃)) EQ9.

Generalizing further, for n=4

K_{T}(t)<(ê(t₁+t₂+t₃+t₄))/(1+(ê−t₁)+(ê−t₂)+(ê−t₃)+(ê−t₄)+ê(−t₁−t₂))+ê(−t₁−t₃))+ê(−t₁−t₄)+ê(−t₂−t₃)+ê(−t₂−t₄)+ê(−t₂−t₃)+ê(−t₃−t₄)+ê(−t₁−t₂−t₃)+ê(−t₁−t₂−t₄)+ê(−t₁−t₃−t₄)+ê(−t₂−t₃−t₄)+ê(−t₁−t₂−t₃−t₄)) EQ10.

A similar mathematical analysis can be readily performed by one who is skilled in the art in the instance where the readout of the assay is an increase in signal.

In another embodiment, synergy can be defined by using an extrapolation of the sigmoidal concentration dependency curve based on the number of signals being co-analyzed. In this embodiment, synergy can be efficiently calculated for any number of potential active moieties each at any concentration, independent of where that concentration is relative to the potential to achieve a signal. As such targets knocked down by putative or existing RNAi or other approaches can thus be screened efficiently through a common mathematic approach to suggest desirable combinations in silico.

For each of these methods the screens as described are run using one or more of these equations to define optimal, improved and/or synergistic combinations. Targets are selected, inhibiting compounds accordingly selected and key combinations validated empirically.

### Phenotypic Assays

The various screens, both in silico and empirical, use of active agents alone in vitro, and testing of RNAco-i complexes in vitro are performed in phenotypic screens. Phenotypic screens involve any assay where the output can be readily quantified. These include, but are not limited to viability, proliferation, apoptosis, migration, differentiation, wound-healing, and angiogenesis assays, as well as other molecular and cellular phenotypic assays including protein or mRNA concentration, localization, immunoblotting, enzymatic activity, redox measurement, phosphylation assessment, FACS, immunohistochemistry, cell counting, radiolabel incorporation, dye exclusion, or growth factor secretion, In a preferred embodiment, the phenotypic screen has a signal that decays with increased activity of the active agent, such as a calorimetric, fluorescent, or luminescent signal which is proportional to cell number, viability, or death (e.g. necrosis or apoptosis) in a cancer cell assay with an effective chemotherapeutic moiety as the agent.

### EXAMPLES

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### Example 1

### Useful Reactive Species for Conjugation to Amines

A variety of reactive functional groups (e.g. on one or more ends of a linear or branched polymer) can be conjugated to an amine-modified siRNA. NHS esters readily react with free amines at pH 7-9 to form stable amide bonds. If desired, the NHS-ester can be joined to the polymer backbone via a carboxylic linker. Certain linkages between the polymer and functional group, for example PEG-succinimidyl succinate, are prone to hydrolytic cleavage (e.g. in the endosome). Others, such as PEG-succinimidyl glutarate, are more resistant to hydrolysis. Nitrophenyl-carbonate groups will react with free amines to form urethane linkages and isocyanate groups will react with free amines to form urea linkages. Aldehydes can be condensed with free amines to form a reversible imine linkage and subsequently reduced with an appropriate reducing agent to form secondary amines.

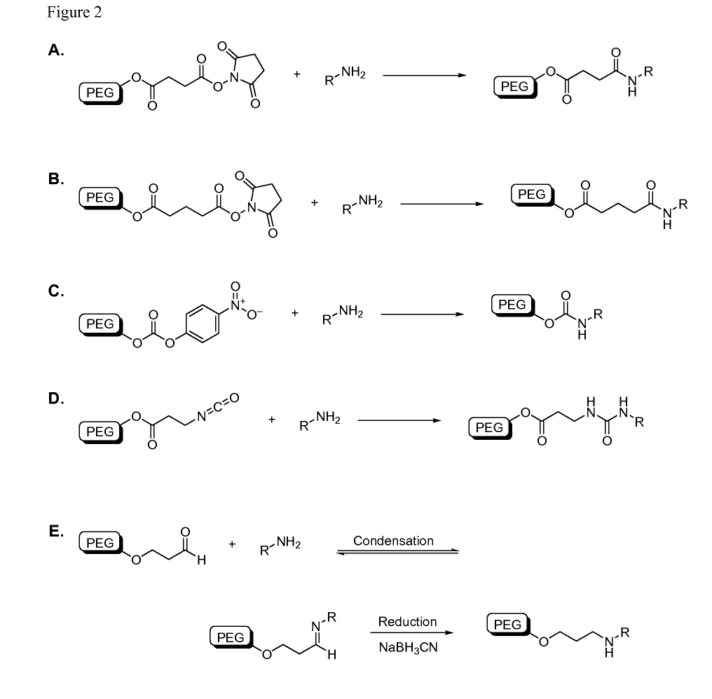

Examples of chemical reactions involving a terminal amine, such as an amine modified siRNA are shown in FIG. 2.

### Example 2

### Useful Reactive Species for Conjugation to Sulfhydryl Groups

A variety of reactive functional groups (e.g. on one or more ends of a linear or branched polymer) can be conjugated to a thiol-modified siRNA. Maleimide groups react with high specificity with sulfhydryl groups (—SH) between pH 6.5 and 7.5 to form a stable thioether bond. Vinylsulfone is another functional group that reacts with sulfhydryl groups to form a stable thiother bond. Orthopyridyl disulfide groups react with sulfhydryl groups to form a disulfide bond, which is a reducible bond optionally subject to disruption within acidic environments, such as the endosome. An acrylate group will react with a sulfhydryl group to generate a similarly acid-labile B-thiopropionate linkage. In addition, an iodoacetimide functional group will react with a sulfhydryl group to form a stable thioether bond.

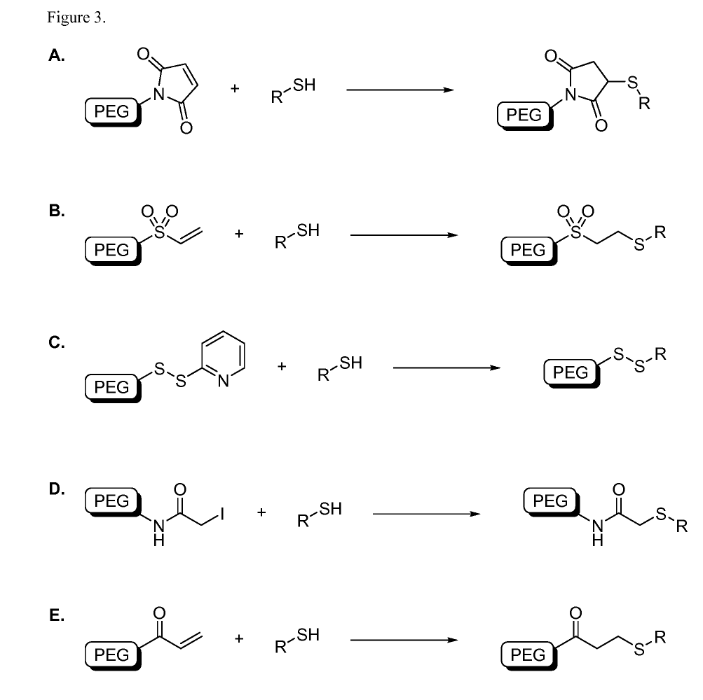

Examples of chemical reactions involving a terminal thiol, such as a thiol modified siRNA are shown in FIG. 3.

### Example 3

### Multiple siRNA Delivery Via Polymeric Linking Agents with Functionalized Monomeric Units

Polymeric linking groups with multiple functionalities, i.e. a chemical functionality on each repeating monomeric unit, can be converted to express a usable functionality. Linking units expressing primary or secondary amine functionalities can be converted to the corresponding iodoacetamide using iodoacetyl chloride, maleimide by reaction with maleic anhydride, 4-nitrophenyl urethane using 4-nitrophenyl chloroformate, acrylamide using acryloyl chloride, or NHS ester using a bis-NHS ester linking agent such as bis(N-hydroxysuccinimidyl) succinate. Linking units expressing carboxylic acid functionalities can be coupled to a primary amine using standard peptide coupling chemistry, or transformed to the NHS ester using standard peptide coupling chemistry. Linking units expressing primary alcohol functionalities can be oxidized (with a reagent that is inert to the polymeric main chain) to the carboxylic acid or converted to a 4-nitrophenyl carbonate functionality using 4-nitrophenyl chloroformate. Primary alcohols can also be converted to the corresponding bromide using reagents such as phosphorus pentabromide or triphenylphosphine and carbon tetrabromide. The primary bromide can then be converted to amine or maleimide using standard sn2 chemistry. Once properly functionalized, the polymeric linking unit can then be reacted with siRNA compounds carrying the appropriate functionality and a variation of specific sequences.

### Example 4

### Homobifunctional PEG Linking Two Different siRNAs Via Stable Thioether Linkage

Homobifunctional PEG maleimide (MAL-PEG-MAL) with an average molecular weight of 3400 Da is purchased through SunBio. 5′-thiol modified siRNA is purchased from Dharmacon using a standard 5′ C6-SH linker as shown below.

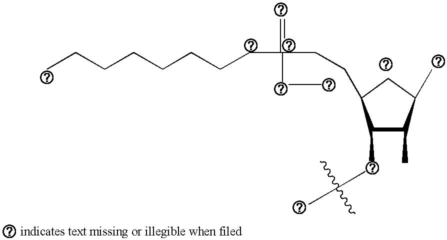

siRNA.EGFR.1 (thiol modified)
S:
HS-5′-GGCACGAGUAACAAGCUCATT-3′
AS:
3′-TTCCGUGCUCAUUGUUCGAGU-5′
siRNA.PI3K.2 (thiol modified)
S:
HS-5′-AAAAUGGCUUUGAAUCUUUGG-3′
AS:
3′-TTUUUUACCGAAACUUAGAAA-5′

The individual siRNAs are pre-annealed by Dharamacon. Upon receipt, siRNAs are resuspended to 100 μM in using the manufacturer's recommended resuspension buffer (20 mM KCl, 6 mM HEPES-pH 7.5, 0.2 mM MgCl₂). An equal volume of siRNA.EGFR.1 and siRNA.PI3K.2 are mixed, generating an equimolar mixture of the two siRNA species (50 μM ea). To 100 μl of this mixture, 30 μl of 100 mM dithiothretal (DTT) is added and incubated overnight at room temperature to cleave any homodimers. The mal-PEG-mal is resuspended in an RNase-free, degassed dimethyl formamide (DMF) to obtain a 2.5 mM solution buffered to pH 6.0-6.5. The DTT is removed from the siRNA samples via a MicroSpin G-25 column (GE Healthcare), which is centrifuged at 735×g in a microcentrifuge for 2 min at room temperature. Following purification, the concentration of the siRNA is determined using a Nanodrop Spectrophotomer (Thermo-Fisher Scientific). To minimize formation of siRNA heterodimers, a 5-fold excess of mal-PEG-mal is first added to an RNase-free tube flooded with argon. Next, the siRNA mixture is pipetted into the mal-PEG-mal tube. Reactions are incubated at 37 C for 2 hrs. The reaction mixture is diluted with RNase-free water to a total volume of 2 ml, then filtered through an addition MicroSpin G-25 column. The siRNA.EGFR.1-PEG-siRNA.PI3K.2 conjugates are purified using HPLC (solvent A: 0.1M tetraethylammonium acetate pH 6.9 in RNase-free water, Solvent B: acetonitrile) followed by microdialysis or a final MicroSpin desalting step.

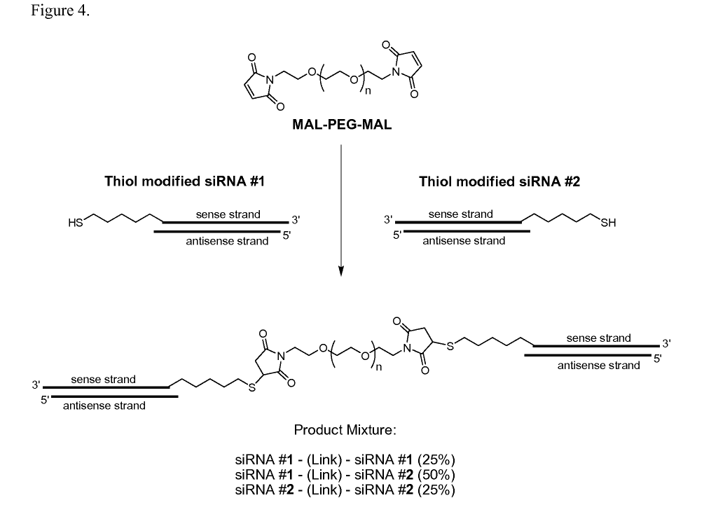

Asymmetric and symmetric formation of a bis-siRNA unit is shown in FIG. 4.

### Example 5

### Homobifunctional PEG Linking Two Different siRNAs Via Acid-Labile B-Thiopropionate Linkage

Homobifunctional PEG acrylate (Acryl-PEG-Acryl) with an average molecular weight of 3400 Da is purchased through Creative PEGWorks. 5′-thiol modified siRNA is purchased from Dharmacon using a standard 5′ C6-SH linker as shown above.

The individual siRNA strands of the siRNAs are ordered separately (e.g. not pre-annealed). To a mixture of the 5′-thiol-modified sense strands of siRNA.EGFR.1 and siRNA.PI3K.2 (15 nmol ea, approximately 97 μg) and excess Ac-PEG-Ac (300 nmole) in 10 mM Tris-HCl buffer pH 8.0 (300 μl), a solution of triphenylphosphine in DMF (1 mM, 60 μl, 2 eq) is added, and incubated for 48 hrs in the dark at room temperature. Following the Michael reaction, the conjugated polymer is filtered and purified on an anion exchange column, eluted with 10 mM Tris-HCl buffer (pH 7.4) using an NaCl gradient ranging from 0 to 0.7 M. Additional purification was carried out via microdialysis against distilled, deionized water (MW cutoff 3500) and then freeze dried. Subsequently, the unmodified antisense siRNA.EGFR.1 and siRNA.PI3K.2 strands (25 μM) are mixed with the reconstituted conjugate (50 μM) in annealing buffer (10 mM Tris-HCl pH 7.4, 50 mM NaCl, and 1 mM EDTA), heat denatured at 95 C for 3 minutes and slow cooled to room temperature.

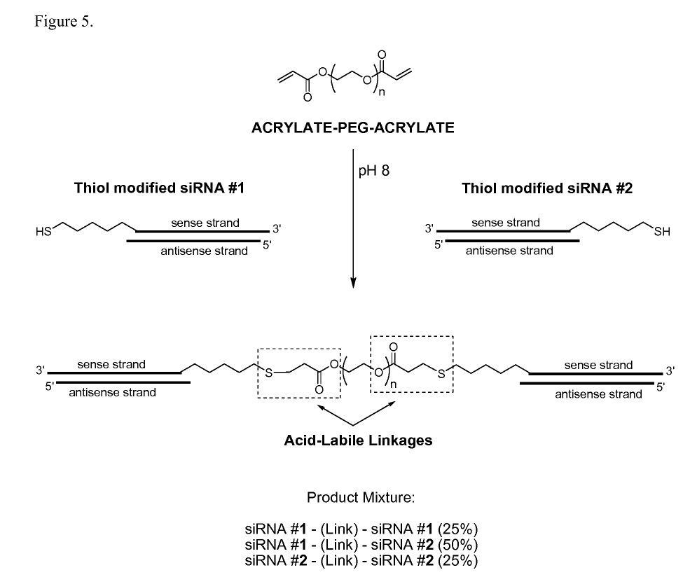

Acid labile thioether linkages made with bis-acrylate PEG moieties are shown in FIG. 5.

### Example 6

### Homobifunctional PEG Linking Two Different siRNAs Via Amide Linkage

Homobifunctional succinimidyl glutarate-PEG-succinimidyl glutarate (NHS-PEG-NHS) with an average molecular weight of 3400 Da is purchased through Creative PEGWorks. 5′-amine modified siRNA is purchased from Dharmacon using a standard 5′-amino (6-carbon) linker as shown below.

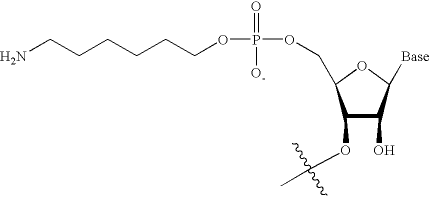

siRNA.survivin.3 (amine modified)
S:
H₂N-5′-GCAAAGGAAACCAACAAUATT-3′
AS:
3′-TTCGUUUCCUUUGGUUGUUAU-5′
siRNA.c-Myc.4 (amine modified)
S:
H₂N-5′-GGUCAGAGUCUGGAUCACCTT-3′
AS:
3′-TTCCAGUCUCAGACCUAGUGG-5′

The respective siRNA duplexes are pre-annealed, then equimolar aliquots (30 nmol ea) are conjugated to NHS-PEG-NHS (30 nmole) in phosphate-buffered saline, pH 8.0. The NHS-PEG-NHS is reconstituted in anhydrous DMSO, not DMF which frequently contains trace quantities of dimethyl amine. The conjugation reaction is incubated for 2 hrs at room temperature, and the siRNA.survivin.3-PEG-siRNA-c-Myc.4 conjugate is subsequently purified via HPLC.

### Example 7

### Homobifunctional PEG Linking Two Different siRNAs Via Amide Linkage

Homobifunctional p-Nitrophenyl carbonate (NPC) PEG (NPC-PEG-NPC) with an average molecular weight of 3400 Da is purchased through SunBio. 3′-amine modified siRNA is purchased from Dharmacon using a standard 3′-amino (6-carbon) linker.

siRNA.survivin.3 (amine modified)
S:
5′-GCAAAGGAAACCAACAAUATT-3′-C₆—NH₂
AS:
3′-TTCGUUUCCUUUGGUUGUUAU-5′
siRNA.c-Myc.4 (amine modified)
S:
5′-GGUCAGAGUCUGGAUCACCTT-3′-C₆—NH₂
AS:
3′-TTCCAGUCUCAGACCUAGUGG-5′

The respective siRNA duplexes are pre-annealed, then equimolar aliquots (30 nmol ea) are conjugated to NPC-PEG-NPC (30 nmole) in phosphate-buffered saline, pH 8.0. The NPC-PEG-NPC is reconstituted in anhydrous DMSO, not DMF which frequently contains trace quantities of dimethyl amine. The conjugation reaction is incubated for 2 hrs at room temperature, and the urethane linked siRNA.survivin.3-PEG-siRNA.c-Myc.4 conjugates are subsequently purified via HPLC followed by dialysis.

### Example 8

### Heterobifunctional PEG Linking Two Different siRNAs Via an Amide and Thioether Linkage

Heterobifunctional maleimide-PEG-NHS (MAL-PEG-NHS) with an average molecular weight of 3500 Da is purchased through JenKem Technologies USA. 5′-thiol modified siRNA.EGFR.1 is purchased from Dharmacon using a standard 5′ C6-SH linker. 5′-amine modified siRNA.survivin.3 is purchased from Dharmacon using a standard 5′-amino (6-carbon) linker.

siRNA.EGFR.1 (thiol modified)
S:
HS-5′-GGCACGAGUAACAAGCUCATT-3′
AS:
3′-TTCCGUGCUCAUUGUUCGAGU-5′
siRNA.survivin.3 (amine modified)
S:
H₂N-C₆-5′-GCAAAGGAAACCAACAAUATT-3′
AS:
3′-TTCGUUUCCUUUGGUUGUUAU-5′

The respective siRNA duplexes are pre-annealed. The thiol-modified siRNA.EGFR.1 duplex is reduced via DTT treatment, as described in Example 4. Following purification through a MicroSpin G-25 column, an equal amount of siRNA.EGFR.1 and siRNA.survivin.3 (30 nmole) are reacted with the maleimide-PEG-NHS ester bifunctional linker (3500 MW; 15 nmole) in RNAse-free phosphate buffered saline, pH 7.4 at 4 C overnight in the dark. Employing a two-fold excess of siRNA helps efficiently drive the heterobifunctional coupling. By virtue of the heterobifunctional reactive groups utilized in the present method, only conjugates with a precisely defined siRNA.EGFR.1-PEG-siRNA.survivin.3 composition are formed.

### Example 9

### Homomultifunctional PEG Linking 4 Different siRNAs Via Reversible B-Thiopropionate Linkage

Homomultifunctional PEG comprising in the form of a 4-arm star-like PEG-acrylate, with an average MW of 10 kDa is purchased through Creative PEGWorks. 5′-thiol modified siRNAs are synthesized by Dharmacon.

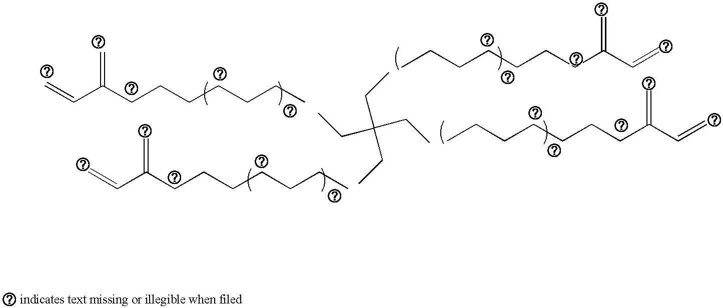

siRNA.EGFR.1 (thiol modified)
S:
SH-5′-GGCACGAGUAACAAGCUCATT-3′
AS:
3′-TTCCGUGCUCAUUGUUCGAGU-5′
siRNA.PI3K.2 (thiol modified)
S:
SH-5′-5′-AAAAUGGCUUUGAAUCUUUGG-3′
AS:
3′-TTUUUUACCGAAACUUAGAAA-5′
siRNA.survivin.3 (thiol modified)
S:
HS-5′-GCAAAGGAAACCAACAAUATT-3′
AS:
3′-TTCGUUUCCUUUGGUUGUUAU-5′
siRNA.c-Myc.4 (thiol modified)
S:
HS-5′-GGUCAGAGUCUGGAUCACCTT-3′
AS:
3′-TTCCAGUCUCAGACCUAGUGG-5′

5′-thiol modified siRNA is purchased from Dharmacon using a standard 5′ C6-SH linker as shown above. The individual siRNA strands of the siRNAs are ordered separately (e.g. not pre-annealed). To a mixture of the 5′-thiol-modified sense strands of siRNA.EGFR.1, siRNA.PI3K.2, siRNA.survivin.3, and siRNA.c-Myc.4 (15 nmol eq) and excess 4-arm PEG-Acryl (300 nmole) in 10 mM Tris-HCl buffer pH 8.0 (300 μl), a solution of triphenylphosphine in DMF (1 mM, 60 μl, 2 eq) is added, and incubated for 48 hrs in the dark at room temperature. Following the Michael reaction, the conjugated polymer is filtered and purified on an anion exchange column, eluted with 10 mM Tris-HCl buffer (pH 7.4) using an NaCl gradient ranging from 0 to 0.7 M. Additional purification is carried out via microdialysis against distilled, deionized water (MW cut-off=3500) and then freeze dried. Subsequently, the unmodified antisense siRNA.EGFR.1, siRNA.PI3K.2, siRNA.survivin.3, and siRNA.c-Myc.4 strands (12.5 μM) are mixed with the reconstituted conjugate (50 μM) in annealing buffer (10 mM Tris-HCl pH 7.4, 50 mM NaCl, and 1 mM EDTA), heat denatured at 95° C. for 3 minutes and slow cooled to room temperature.

### Example 10

### Homomultifunctional PEG Linking 6 Different siRNAs Via Amide Bond

Homomultifunctional PEG comprising in the form of a 6-arm PEG-succinimidyl glutarate, with an average MW of 10 kDa is purchased through Sun Bio. 3′-amine modified siRNA is purchased from Dharmacon using a standard 3′-amino (6-carbon) linker.

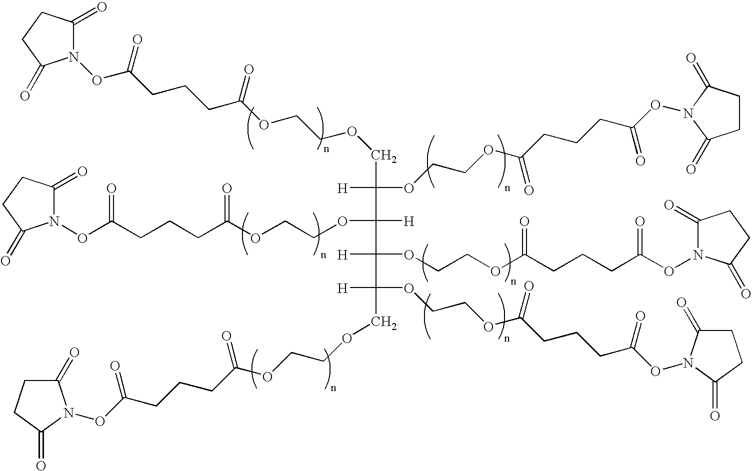

siRNA.EGFR.1 (amine modified)
S:
5′-GGCACGAGUAACAAGCUCATT-3′-C₆—NH₂
AS:
3′-TTCCGUGCUCAUUGUUCGAGU-5′
siRNA.PI3K.2 (amine modified)
S:
5′-AAAAUGGCUUUGAAUCUUUGG-3′-C₆—NH₂
AS:
3′-TTUUUUACCGAAACUUAGAAA-5′
siRNA.survivin.3 (amine modified)
S:
5′-GCAAAGGAAACCAACAAUATT-3′-C₆—NH₂
AS:
3′-TTCGUUUCCUUUGGUUGUUAU-5′
siRNA.c-Myc.4 (amine modified)
S:
5′-GGUCAGAGUCUGGAUCACCTT-3′-C₆—NH₂
AS:
3′-TTCCAGUCUCAGACCUAGUGG-5′
siRNA.Met.5 (amine modified)
S:
5′-ACUCAGAAGAGAUAGUAAUGCUCAG-3′-C₆—NH₂
AS:
3′-UUUGAGUCUUCUCUAUCAUUACGAGUC-5
siRNA.PDGFRA.6 (amine modified)
S:
5′-GCCAAUUAGACUUGAAAUACGUUTG-3′-C₆—NH₂
AS:
3′-CUCGGUUAAUCUGAACUUUAUGCAAAC-5

The respective siRNA duplexes are pre-annealed, then equimolar aliquots (15 nmol ea) are conjugated to NHS-PEG-NHS (30 nmole) in phosphate-buffered saline, pH 8.0. The 6-arm PEG-NHS ester is reconstituted in anhydrous DMSO, not DMF which frequently contains trace quantities of dimethyl amine. The conjugation reaction is incubated for 2 hrs at room temperature, and subsequently purified via HPLC and dialysis.

### Example 11

### Heterobifunctional PEG Linking 3 siRNAs

Heterobifunctional maleimide-PEG-NHS (MAL-PEG-NHS) with an average molecular weight of 3500 Da is purchased through JenKem Technologies USA. 5′-thiol and 3′-amine sense strand modified siRNA#1 is purchased from Dharmacon using a standard 5′ C6-SH linker and a 3′-amino (6-carbon linker). 5′-thiol modified siRNA #2 is purchased from Dharmacon using a 5′ C6-SH linker. 5′-amine modified siRNA #3 is purchased from Dharmacon using a standard 5′-amino (6-carbon) linker.

siRNA.EGFR.1 (thiol and amine modified)
S:
HS-5′-GGCACGAGUAACAAGCUCATT-3′-C₆—NH₂
AS:
3′-TTCCGUGCUCAUUGUUCGAGU-5′
siRNA.PI3K.2 (thiol modified)
S:
HS-5′-AAAAUGGCUUUGAAUCUUUGG-3′
AS:
3′-TTUUUUACCGAAACUUAGAAA-5′
siRNA.survivin.3 (amine modified)
S:
H₂N—C₆-5′-GCAAAGGAAACCAACAAUATT-3′
AS:
3′-TTCGUUUCCUUUGGUUGUUAU-5′

The respective siRNA duplexes are pre-annealed. The thiol-modified siRNA.EGFR.1 and siRNA.PI3K.2 duplexes are reduced via DTT treatment, as described in Example 4. Following purification through a MicroSpin G-25 column, an equal amount of siRNA.PI3K.2 (30 nmole) is reacted with maleimide-PEG-NHS ester bifunctional linker (3500 MW; 60 nmole) in RNAse-free phosphate buffered saline, pH 7.4 at 4° C. overnight in the dark. In a separate reaction, the amine-modified siRNA.survivin.3 (30 nmole) is reacted with maleimide-PEG-NHS ester (60 nmole) in RNAse-free phosphate buffered saline, pH 7.4 at 4° C. overnight in the dark. Subsequently, the siRNA.PI3K.2 conjugate (linked to PEG-NHS via a thioether linkage) and siRNA.survivin.3 conjugate (linked to MAL-PEG via a carboxyamide linkage) are purified by HPLC and dialyzed. Finally, the two purified conjugates (15 nmole ea) are combined with siRNA.EGFR.1 (30 nmole) in RNAse-free phosphate buffered saline, pH 7.4 at 4 C overnight in the dark. The final product (siRNA.PI3K.2-PEG-siRNA.EGFR.1-PEG-siRNA.survivin.3) is purified by HPLC and dialyzed.

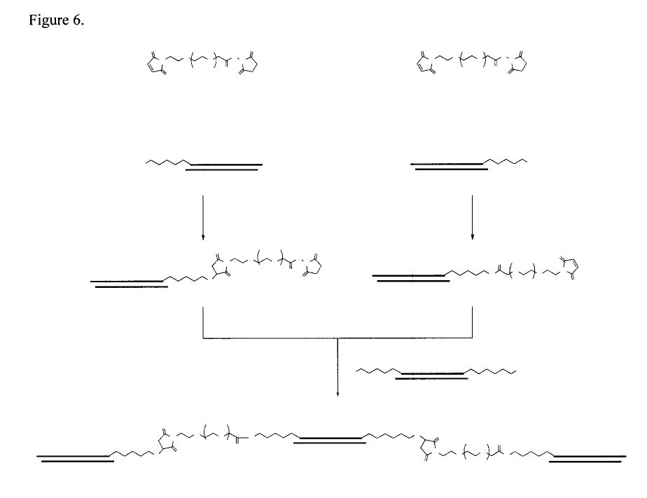

FIG. 6 shows the methodology for making a specific tri-siRNA compound.

### Example 12

### Poly-(Beta-Amino Esters) Polymers Linking Two siRNAs

1,4-butanediol diacrylate is purchased from the Sartomer Company (Exton, Pa.). 5′-amine modified siRNA.EGFR.1 and siRNA.Met.5 are purchased from Dharmacon using a standard 5′-amino (6-carbon) linker.

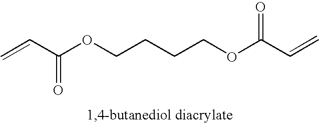

siRNA.EGFR.1 (amine modified)
S:
H₂N—C₆-5′-GGCACGAGUAACAAGCUCATT-3′
AS:
3′-TTCCGUGCUCAUUGUUCGAGU-5′
siRNA.Met.5 (amine modified)
S:
H₂N—C₆-5′-ACUCAGAAGAGAUAGUAAUGCUCAG-3′
AS:
3′-UUUGAGUCUUCUCUAUCAUUACGAGUC-5

To synthesize the polymers, an equimolar mixture of annealed amine-modified siRNA.EGFR.1 and siRNA.Met.5 (100 nmol ea) is combined with 200 nmole of 1,4-butanediol diacrylate (reconstituted in DMSO). The combined monomers are incubated at 50° C. for 2-48 hrs. The length of the resultant polymers is directly proportional to the duration of the incubation period. Thus, for applications in which short polymers are desirable (e.g. one, two, three, four, or five repeats comprising siRNA.EGFR.1 and siRNA.Met.5), incubations are typically performed for 2-4 hrs. For applications in which long polymers are desirable (e.g. ten, eleven, twelve, thirteen, fourteen, fifteen, or more repeats comprising siRNA.EGFR.1 and siRNA.Met.5), incubations are typically performed for 24-48 hrs or more. After the incubation is complete, the polymer is slowly cooled to room temperature and dripped slowly into vigorously stirring diethyl ether or hexanes. The polymer is collected and dried under vacuum prior to analysis or use.

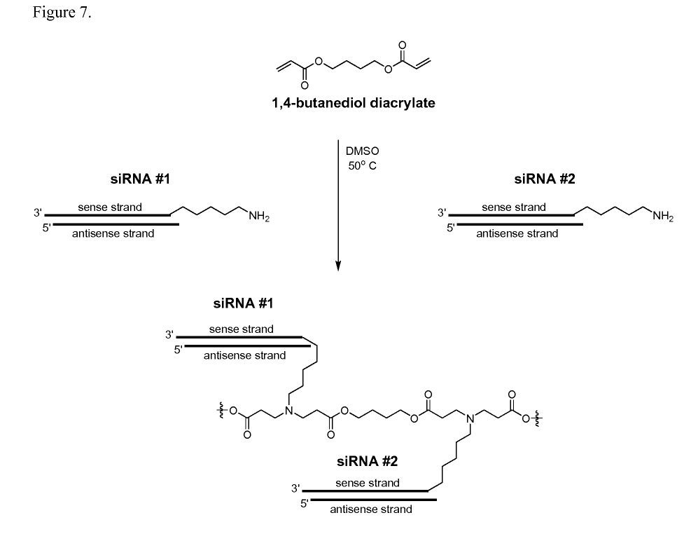

siRNAs expressing amine functionalities can also be co-polymerized with 1,4-butanediol diacrylate as shown in FIG. 7.

### Example 13

### Fixed Formulation Poly-(Beta-Amino Esters) Polymers Linking Two siRNAs

1,4-butanediol diacrylate is purchased from the Sartomer Company (Exton, Pa.). The sense strand of siRNA.EGFR.1 is synthesized by Dharmacon with a fluorescein (FAM) label on the 5′ end and a 3′-amino (6-carbon linker). The sense strand of siRNA.PI3K.2 is synthesized by Dharmacon with a DY547 (a Cy3 alternative dye, hereafter referred to as Cy3) label on the 5′ end and a 3′-amino (6-carbon linker). The corresponding antisense strands are unmodified.

siRNA.EGFR.1 (FAM and amino-modified)
S:
FAM-5′-GGCACGAGUAACAAGCUCATT-3′-C₆—NH₂
AS:
3′-TTCCGUGCUCAUUGUUCGAGU-5′
siRNA.PI3K.2 (Cy3 modified)
S:
Cy3-5′-AAAAUGGCUUUGAAUCUUUGG-3′-C₆—NH₂
AS:
3′-TTUUUUACCGAAACUUAGAAA-5′

To synthesize the polymers, an equimolar mixture of annealed FAM and amine-modified siRNA.EGFR.1 and Cy3 and amine-modified siRNA.PI3K.2 (100 nmol ea) is combined with 200 nmole of 1,4-butanediol diacrylate (reconstituted in DMSO). The combined monomers are incubated at 50° C. for 12 hrs. In a separate reaction, FAM and amine-modified siRNA.EGFR.1 (25 nmole) and Cy3 and amine-modified siRNA.PI3K.2 (100 nmol ea) is combined with 125 nmole of 1,4-butanediol diacrylate (reconstituted in DMSO). The combined monomers are incubated at 50° C. for 12 hrs. After the incubation is complete, the polymer is slowly cooled to room temperature and dripped slowly into vigorously stirring diethyl ether or hexanes. The polymer is collected and dried under vacuum prior to analysis or use. An aliquot of each polymer is reconstituted in THF and analyzed on a Synergy™ 4 Multi-Mode Microplate Reader with Hybrid Technology™ (BioTek Instruments, Winooski, Vt.). A standard curve of known quantities of FAM- and Cy3-conjugated siRNA (or dye alone) is developed and analyzed in parallel to facilitate quantization. As expected, the polymer created with equimolar quantities of siRNA.EGFR.1 and siRNA.PI3K.2 are found to exhibit a 1:1 ratio of FAM:Cy₃, whereas the second formulation exhibits a 1:5 ratio of FAM:Cy₃.

### Example 14

### PEG-PLGA-PEG Triblock Polymers Linking Two siRNAs

PLGA with an average molecular weight of 5000 Da is purchased from Wako Chemicals USA (Richmond, Va.). The carboxylate end is functionalized with ethanolamine using standard EDC/HOBt coupling chemistry, generating HO-PLGA-OH. Next HO-PLGA-OH is reacted with p-toluenesulfonyl chloride (TsCl) as a tosylating agent and triethyleneamine (TEA) in the presence of dichloromethane to produce TsO-PLGA-OTs, which is subsequently reacted with ammonia water to produce free amine groups in the form of NH₂—PLGA-NH₂. This scheme has generally been used to functionalize a PEG polymer (see Kwang N et al, U.S. Pat. No. 6,828,401 “Preparation method of PEG-maleimide derivatives”).

Next, heterobifunctional HOOC-PEG-Mal, with an average molecular weight of 3000 is purchased from IRIS Biotech GbmH (Marktredwitz, Germany). The HOOC-PEG-Mal is conjugated to 5′-thiol sense strand modified siRNA.EGFR.1 and siRNA.PI3K.2 as described above. The carboxylic acid group of HOOC-PEG-siRNA.EGFR.1 and HOOC-PEG-siRNA.P13K.2 are subsequently activated with an NHS-ester group via addition of N-hydroxysuccinimide (NHS) in dry methylene chloride and 1-ethyl-3-[3-dimethylaminopropyl]carbodiimide hydrochloride (EDC).

Finally, the amine-modified PLGA (200 nmole) is reacted with an equimolar mixture of NHS-PEG-siRNA EGFR.1 (100 nmole) and NHS-PEG-siRNA.PI3K.2 (100 nmole) for 2 hrs at room temperature to form the siRNA.EGFR.1-PEG-PLGA-PEG-siRNA.P13K.2 triblock polymer. The conjugate is subsequently purified by HPLC and dialysed prior to use.

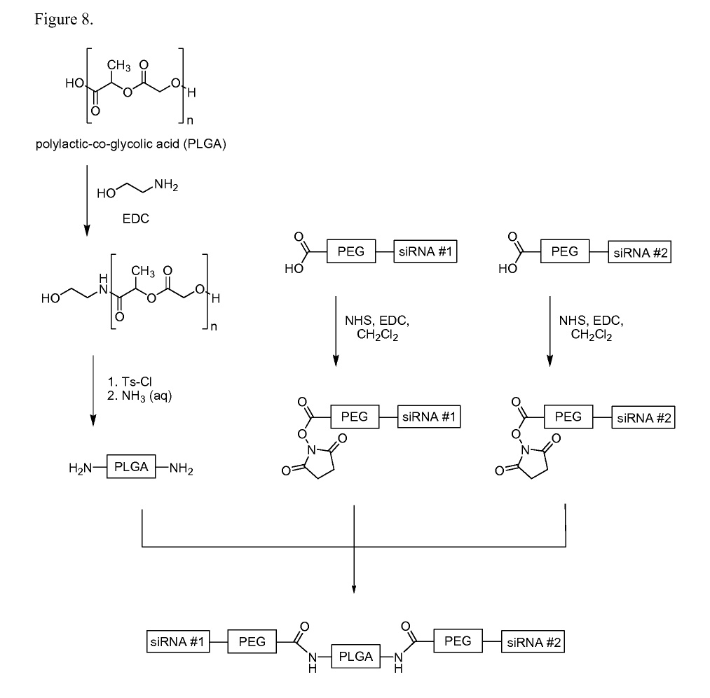

A method of obtaining a block polymer linking unit is shown in FIG. 8.

### Example 15

### Peptide-Labeled siRNAs Linked Via PEG

Both polypeptides and siRNA are prepared using standard solid phase synthesis methods. siRNA.PGFRA.6 and siRNA.PDGFRB.7 are synthesized with 5′-thiol and 3′-amine modified sense strand by Dharmacon. Labeling polypeptide moieties are synthesized by AnaSpec (San Jose, Calif.), including a hexahistidine tag (HHHHHH) and the FLAG tag epitope (DYKDDDK). Maleimido modified peptides are prepared by coupling three (3) equivalents of 3-maleiimidopropionic acid and HCTU in the presence of 6 equivalents of N-methylmorpholine to the N-terminus of the peptide resin (described in 20060035815).

siRNA.PDGFRA.6
S:
HS-5′-GCCAAUUAGACUUGAAAUACGUUTG-3′-C₆—NH₂
AS:
3′-CUCGGUUAAUCUGAACUUUAUGCAAAC-5
siRNA.PDGFRB.7
S:
HS-5′-GCACUAACAUUCUAGAGUAUUCCAG-3′-C₆—NH₂
AS:
3′-GUCGUGAUUGUAAGAUCUCAUAAGGUC-5

Purified reduced siRNA.PGFRA.6 sense strand is dissolved in 0.1 M triethylamine acetate (TEAA) buffer pH 7.0 and then maleimido-modified HHHHHH is added to the oligonucleotide solution. After addition of peptide a precipitate is formed which disappears upon the addition of 150 μl of 75% CH₃CN/0.1M TEAA. After stirring overnight at room temperature, the resulting conjugate is purified by reverse phase HPLC on XTerra.RTM.MS Cl₈ 4.6.times.50 mm column using a linear gradient from 0-30% of CH.sub.3CN in 0.1M TEAA buffer pH 7 within 20 min and 100% C within next 5 min (t.sub.r=21.007 min). The amount of the conjugate is determined by spectrophotometry based on the calculated molar absorption coefficient at λ=260 nm. MALDI mass spectrometric analysis showed that the peak observed for the conjugate matches the calculated mass. Yields are typically 25%-50%. Next, the same procedure is applied to conjugate maleimido-modified DYKDDDK to the purified reduced siRNA.PDGFRB.7 sense strand.

The peptide conjugate sense strands and complementary antisense strands are annealed in 50 mM potassium acetate, 1 mM magnesium acetate and 15 mM HEPES pH 7.4 by heating at 90° C. for 3 minutes followed by slow cooling to room temperature. Next, the peptide labeled 5′-conjugated siRNA.PGFRA.6 and siRNA.PDGFRB.7 (still possessing the remaining 3′-amine) are conjugated to homobifunctional p-Nitrophenyl carbonate (NPC) PEG (NPC-PEG-NPC) with an average molecular weight of 3400 Da (purchased through SunBio) as described above in Example 7. The resultant conjugates are subsequently isolated using metal- and immunoaffinity chromatography. First, the conjugates are purified using nickel-nitrilotriacetic acid (Ni-NTA) Superflow resin (Qiagen). The eluted conjugates, which are now known to contain HHHHHH-conjugated siRNA.PGFRA.6, are subsequently dialyzed to remove the imidazole. Next, the eluted conjugates are purified using anti-FLAG M2 affinity gel (Sigma-Aldrich, St. Louis, Mo.). Conjugates that bind to the anti-FLAG M2 affinity resin must contain both the HHHHHH-conjugated siRNA.PGFRA.6 and the FLAG-epitope-conjugated siRNA.PDGFRB.7 linked through a common PEG moiety. The eluted conjugates need no further purification, but are dialyzed to remove free peptide and remaining salts. Finally, enzyme-linked immunosorbent assays (ELISAs) are performed using anti-6×-His and anti-FLAG tag antibodies to confirm the identity and stoichiometry of the conjugates. A standard curve is generated using free 6×-His and FLAG peptides.

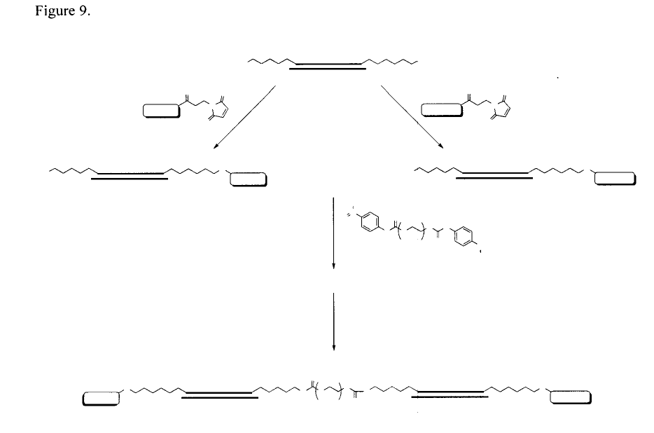

An example of one method of purifying mixtures of linked siRNAs using protein is shown in FIG. 9.

### Example 16

### Fluorescence-Based Purification of Fixed Formulation siRNA Conjugates

1,4-butanediol diacrylate is purchased from the Sartomer Company (Exton, Pa.). The sense strand of siRNA.EGFR.1 is synthesized by Dharmacon with 5′-thiol and 3′-amine modifications. The sense strand of siRNA.PI3K.2 is synthesized by Dharmacon with a DY547 (a Cy3 alternative dye, hereafter referred to as Cy3) label on the 5′ end and a 3′-amino (6-carbon linker). The corresponding antisense strands are unmodified. A labeling peptide comprising a Factor Xa cleavage site (IEGR) and 6×-His tag (IEGRHHHHHH) with a C-terminal Cy5 conjugate is synthesized by Anaspec. The labeling peptide is maleimido modified by coupling three (3) equivalents of 3-maleiimidopropionic acid and HCTU in the presence of 6 equivalents of N-methylmorpholine to the N-terminus of the peptide.

siRNA.EGFR.1 (thiol and amine modified)
S:
HS-5′-GGCACGAGUAACAAGCUCATT-3′-C₆—NH₂
AS:
3′-TTCCGUGCUCAUUGUUCGAGU-5′
siRNA.PI3K.2 (Cy3 and amine modified)
S:
Cy3-5′-AAAAUGGCUUUGAAUCUUUGG-3′-C₆—NH₂
AS:
3′-TTUUUUACCGAAACUUAGAAA-5′

Purified reduced siRNA.EGFR.1 sense strand is dissolved in 0.1 M triethylamine acetate (TEAA) buffer pH 7.0 and then maleimido-modified IEGRHHHHHH-Cy3 is added to the oligonucleotide solution. After addition of peptide a precipitate is formed which disappears upon the addition of 150 μl of 75% CH₃CN/0.1M TEAA. After stirring overnight at room temperature, the resulting conjugate is purified by RP HPLC on XTerra.RTM.MS C₁₈ 4.6.times.50 mm column using a linear gradient from 0-30% of CH.sub.3CN in 0.1M TEAA buffer pH 7 within 20 min and 100% C within next 5 min (t.sub.r=21.007 min). The amount of the conjugate is determined by spectrophotometry based on the calculated molar absorption coefficient at X=260 nm. MALDI mass spectrometric analysis showed that the peak observed for the conjugate matches the calculated mass.

To synthesize a poly(beta-amino ester) polymer, an equimolar mixture of annealed siRNA.EGFR.1-IEGRHHHHHH-Cy5 conjugate (100 nmole) and Cy3 and amine-modified siRNA.PI3K.2 (100 nmol ea) is combined with 200 nmole of 1,4-butanediol diacrylate (reconstituted in DMSO). The combined monomers are incubated at 50° C. for 12 hrs. After the incubation is complete, the polymer is slowly cooled to room temperature and dripped slowly into vigorously stirring diethyl ether or hexanes. The polymer is collected and dried under vacuum prior. Next, the polymer is immobilized onto Ni-NTA agarose beads (which captures only polymers containing siRNA.EGFR.1). The beads are subsequently sorted using by Cy3 and Cy5 channels using a FACSCaliber flow cytometer (Becton and Dickinson). Beads containing the desired ratio of Cy3:Cy5 are collected, and the polymer conjugates are subsequently cleaved from the beads using Factor Xa protease (Pierce, Rockford, Ill.).

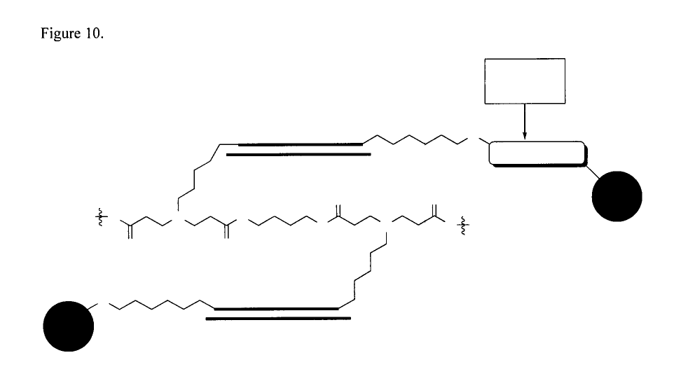

Co-polymers of two siRNA strands expressing a specific ratio of one siRNA strand to other can be achieved by the co-polymerization of amine functionalized siRNA strands using 1,4-butanediol diacrylate, as shown in FIG. 10.

### Example 17

### siRNA-PEG-Taxol Conjugate

The 2′-hydroxyl on Taxol (Sigma Chemical) was first protected using the [(2,2,2-trichloroethyl)oxy]carbonyl, or ‘troc’ protective group. Taxol (50 mg) in chloroform (5 ml) and pyridine (0.1 ml) was cooled to −20° C. and treated with 2,2,2-trichloroethyl chloroformate (0.008 ml) for 45 minutes. Workup by standard methods yielded the 2′-troc derivative together with small amounts of taxol and 2′,7-bis troc taxol. The 2′-troc product is isolated by TLC with ethyl acetate-hexane (1:1) as solvent: yield 85%. 10 mg (11.7 μmol) if 2′ protected Taxol is combined with a ten-fold excess of 1,1-carbonyldiimidazole (CDI, 18.95 mg, 117 μmol) to obtain a 7′-Taxol-CDI derivative. The reaction is incubated for 2 hrs at room temperature and then extracted three times in water to remove the excess CDI and imidazole formed during the reaction, and subsequently dried over anhydrous sodium sulfate.

Heterobifunctional maleimide-PEGamine (MAL-PEG-NH₂) with an average molecular weight of 3400 Da (Creative PEGWorks) is coupled to 5′-thiol sense strand modified siRNA.survivin.3 as described above in Example 9. The resulting siRNA.survivin.3-PEG-NH₂ (1 μmol) is combined with an equimolar amount of Taxol-CDI (1 μmol) and allowed to react for 2 hours with stirring at room temperature. The 2′-troc modification is subsequently removed by dissolving the conjugate in 2 ml of methanol-acetic acid (9:1) and addition of zinc dust (40 mg). The mixture is stirred for 10 minutes at room temperature, filtered to remove excess zinc, and the siRNA.survivin.3-PEG-Taxol conjugate is precipitated with diethyl ether and dried in vacuo.

siRNA.survivin.3 (thiol modified)
S:
HS-5′-GCAAAGGAAACCAACAAUATT-3′
AS:
3′-TTCGUUUCCUUUGGUUGUUAU-5′

### Example 18

### siRNA-PEG-Camptothecin Conjugate

(S)-(+)-Camptothecin (CPT) is purchased from Sigma Alridch. Heterobifunctional HOOC-PEG-Mal, with an average molecular weight of 3000 is purchased from IRIS Biotech GbmH (Marktredwitz, Germany). The HOOC-PEG-Mal is dried by azeotroping with 75 ml of toluene in a Dean-Stark apparatus for 2 hrs. The remaining toluene is removed under vacuum, and the PEG and CPT are dissolved in 100 ml of methylene chloride with stirring. The solution is cooled to 4° C. in an ice bath. 2-Chloro-1-methylpyridinium iodide and 4-(dimethylamino) pyridine (DMAP) are added, the solution is warmed to room temperature and the reaction is continued for 48 hrs. The organic solution is then washed with 0.5 M HCl (2×25 ml) and dried over MgSO₄. Solvent is removed under vacuum. The product is redissolved in 2 ml of methylene chloride and precipitated upon addition of 200 ml of 2-propanol. The precipitate is collected by filtration and dried under vacuum. The CPT-PEG-Mal conjugate is subsequently reacted with 5′ thiol-modified sense strand sense strand siRNA.survivin.3 as outlined above in Example 17. The conjugate is purified using reverse phase HPLC and dialyzed prior to use.

### Example 19

### Methotrexate-siRNA Conjugate

Methotrexate, purchased from Sigma-Aldrich (1 μmole) is reconstituted in 1 ml of anhydrous DMF. A solution of N-hydroxysuccinimide (2 equivalents, 2 μmole) in 8 mls of anhydrous DMF and a solution of 1,3-dicyclohexylcarbodiimide (2 equivalents, 2 μmole) in 0.75 ml anhydrous DMF are added, and the reaction mixture is stirred in the dark at room temperature for 16 hours under anhydrous conditions. A white precipitate is formed, which is removed by centrifugation. The clear supernatant, containing NHS-ester-activated methotrexate, is gradually added to 3′-amine modified siRNA.c-Myc.4 (Dharmacon) in 2 ml of phosphate-buffered saline, pH 7.2. The solution is mixed at room temperature for 5 hrs and desalted on a MicroSpin Sephadex G-25 column. The void volume is collected, and subsequently dialyzed prior to use.

siRNA.c-Myc.4 (amine modified)
S:
5′-GGUCAGAGUCUGGAUCACCTT-3′-C₆—NH₂
AS:
3′-TTCCAGUCUCAGACCUAGUGG-5′

### Example 20

### Methotrexate Linked to PEG Conjugate Containing Two Distinct siRNAs

Methotrexate, purchased from Sigma-Aldrich (1 μmole) is reconstituted in 1 ml of anhydrous DMF. A solution of N-hydroxysuccinimide (2 equivalents, 2 μmole) in 8 mls of anhydrous DMF and a solution of 1,3-dicyclohexylcarbodiimide (2 equivalents, 2 μmole) in 0.75 ml anhydrous DMF are added, and the reaction mixture is stirred in the dark at room temperature for 16 hours under anhydrous conditions. A white precipitate is formed, which is removed by centrifugation. The clear supernatant, containing NHS-ester-activated methotrexate, is removed.

Heterobifunctional maleimide-PEG-NHS (MAL-PEG-NHS) with an average molecular weight of 3500 Da is purchased through JenKem Technologies USA. The sense strand of siRNA.EGFR.1 is synthesized with a 5′-thiol modified and 3′-amine modification by Dharmacon. 5′-amine modified siRNA.c-Myc.4 is purchased from Dharmacon using a standard 5′-amino (6-carbon) linker.

siRNA.EGFR.1 (thiol and amine modified)
S:
HS-5′-GGCACGAGUAACAAGCUCATT-3′-C₆—NH₂
AS:
3′-TTCCGUGCUCAUUGUUCGAGU-5′
siRNA.c-Myc.4 (amine modified)
S:
H₂N—C₆-5′-GGUCAGAGUCUGGAUCACCTT-3′
AS:
3′-TTCCAGUCUCAGACCUAGUGG-5′

The respective siRNA duplexes are pre-annealed. The thiol-modified siRNA.EGFR.1 duplex is reduced via DTT treatment, as described in Example 3. Following purification through a MicroSpin G-25 column, an equal amount of siRNA.EGFR.1 and siRNA.c-Myc.4 (30 nmole) are reacted with the maleimide-PEG-NHS ester bifunctional linker (3500 MW; 15 nmole) in RNAse-free phosphate buffered saline, pH 7.4 at 4° C. overnight in the dark. Employing a two-fold excess of siRNA helps efficiently drive the heterobifunctional coupling. The methotrexate-NHS-ester supernatant is gradually added to the 3′-amine-modified-siRNA.EGFR.1-PEG-siRNA.c-Myc.4 conjugate. The solution is mixed at room temperature for 5 hrs and desalted on a MicroSpin Sephadex G-25 column. The void volume is collected, and subsequently purified by reverse phase HPLC and dialyzed prior to use.

### Example 21

### Formation of Polyethylene-Imine Nanoparticles

Linear polyethylene imine (PEI) reconstituted to 150 mM (expressed as the concentration of monomer nitrogen residues) (Sigma-Aldrich, USA). Prior to complex formation, the PEI is diluted into a sterile 5% glucose solution. The purified siRNA.EGFR.1-PEG-siRNA.survivin.3 prepared in Example 7 is aliquoted into several microfuge tubes and combined with multiple aliquots of PEI to achieve an N:P ratio of 2, 4, 8, 16, 24, or 32. The N/P ratio is a measure of the ionic balance of the complexes, referring to the number of nitrogen residues of PEI per siRNA phosphate. The reactions are incubated for 30 minutes at room temperature, after which the solution is layered onto media of cell cultures in vitro or administered via systemic or local injection in vivo.

### Example 22

### Co-Encapsulation of Paclitaxel and siRNA in a Cationic Liposome

Cationic liposomes with a total lipid content of 50 mM are prepared by the lipid film method followed by several cycles of extrusion. Briefly, 0.25 mmol 1,2-dioleoyl-3-trimethylammonium propane and 0.235 mmol 1,2-dioleoyl-sn-glycero-3-phosphocholine (both from Avanti Polar Lipids, Alabaster) with or without 15 μmol paclitaxel and/or FAM-labeled siRNA.EGFR.1 (1 μmol) are dissolved in 15 ml chloroform. The respective mixtures are gently warmed to 40° C. in a round-bottomed flask and the solvent is evaporated under vacuum in a rotary evaporator until a thin lipid film is formed. Solvent traces are eliminated by drying the film at 5 mba for 60 min. Multilamellar liposomes form spontaneously upon addition of 10 ml 5% glucose (wt/vol) to the flask. The suspension is left overnight to allow swelling of the liposomes. Next, the suspension is extruded five times in a 10 ml extruder (Lipex, Vancouver, Canada) with a thermobarrel thermostatted at 30° C. The pore size of the polycarbonate membrane (Osmonics, Minnetonka, Minn.) is 200 nm. The resulting suspension is stored at 4° C. under argon. The particle size of the liposomes is analyzed by photon correlation spectroscopy using a Malvern Zetasizer 3000 (Malvern Instruments, Herrenberg, Germany). Typically, the suspensions exhibit a Z average of 180-200 nm. Lipid and paclitaxel concentrations are determined by high performance liquid chromatography using a UV/VIS and fluorescence detector (205 nm for lipids, 227 nm for paclitaxel, and 518 nm for FAM-siRNA). The separation and quantification of the components are carried out using a C₈ LiChrospher 60 RP-select B column (250×4 mm, 5 mm particle size) with a C₁₈ pre-column.

### Example 23

### Therapeutic Antibody-siRNA Conjugates

Purified chimeric monoclonal anti-EGFR (erbitux) is obtained from ImClone Systems (New York, N.Y.). A 6×-His tag peptide is synthesized by AnaSpec (San Jose, Calif.). Sense strand 5′-thiol and 3′-amino modifications siRNA.EGFR.1 is synthesized by Dharmacon. Maleimido modified 6×-His peptide is prepared by coupling three (3) equivalents of 3-maleiimidopropionic acid and HCTU in the presence of 6 equivalents of N-methylmorpholine to the N-terminus of the peptide resin.

Purified reduced siRNA.EGFR.1 sense strand is dissolved in 0.1 M triethylamine acetate (TEAA) buffer pH 7.0 and then maleimido-modified HHHHHH is added to the oligonucleotide solution. After addition of peptide a precipitate is formed which disappears upon the addition of 150 μl of 75% CH₃CN/0.1M TEAA. After stirring overnight at room temperature, the resulting conjugate is purified by reverse phase HPLC on XTerra.RTM.MS Cl₈ 4.6.times.50 mm column using a linear gradient from 0-30% of CH.sub.3CN in 0.1M TEAA buffer pH 7 within 20 min and 100% C within next 5 min (t.sub.r=21.007 min). The amount of the conjugate is determined by spectrophotometry based on the calculated molar absorption coefficient at λ=260 nm. MALDI mass spectrometric analysis showed that the peak observed for the conjugate matches the calculated mass.

The peptide conjugate sense strands and complementary antisense strands are annealed in 50 mM potassium acetate, 1 mM magnesium acetate and 15 mM HEPES pH 7.4 by heating at 90° C. for 3 minutes followed by slow cooling to room temperature. Next, homobifunctional NHS-PEG-NHS with an average molecular weight of 3400 Da (Creative PEGWorks) is reacted with an equimolar mixture of 6×-His-tagged siRNA.EGFR.1 (containing a free 3′ amino group) (1 μmol) and anti-EGFR antibody (1 μmol) in phosphate buffered saline, pH 7.4 for 2 hours at room temperature. Conjugates are purified by reverse phase HPLC. The ratio of siRNA to antibody is calculated based on quantitative ELISA data using anti-6×-His (siRNA.EGFR.1) and anti-Ig (antibody).

### Example 24

### Monitoring siRNA Activity

Typically, the siRNA conjugates are initially tested in vitro via transfection of HeLa cells (purchased from The American Type Culture Collection). In all instances, transfections are performed using the siRNA conjugates alone and with delivery carriers including PEI (from Sigma-Aldrich) and Lipofectamine 2000 (Invitrogen, Carlsbad, Calif.). At 48 hours post-transfection, total RNA samples are isolated using the high throughput MagMAX™-96 Total RNA Isolation Kit (Ambion). RNA yields are quantitated using a NanoDrop spectrophotometer system (Thermo-Fisher Scientific). An equivalent amount of each RNA sample is reverse transcribed using the High Capacity cDNA RT Kit with RNAse Inhibitor (Ambion). Normalizing RNA content prior to reverse transcription minimizes potential misinterpretation of results, which might occur if the siRNA in question causes a change in relative cell number. Using the normalized cDNA reactions, the expression levels of each relevant gene (e.g. the genes targeted by the relevant siRNAs—EGFR, PDGFα, PDGFβ, Met, etc.) are be analyzed via quantitative PCR using Inventories TaqMan Gene Expression Assays (Applied Biosystems) on an ABI-7900 real-time PCR instrument (Applied Biosystems). The 18S rRNA content of each cDNA sample is also analyzed via a TaqMan Gene Expression Assay. The efficiency of gene knockdown is determined using the ΔΔCt method.

In addition, siRNA conjugate activity is also monitored at the protein level via western blotting. Whole cell extracts are prepared 72 hrs post transfection by lysing cells in 50 mM Tris pH 7.5, 300 mM NaCl, 0.5% Triton X-100, containing Complete Protease Inhibitor Cocktail Tablets (Roche Molecular Biochemicals). Protein concentration is determined by BioRad DC assay (BioRad Laboratories, Hercules, Calif.). An equal quantity of protein is analyzed by western blotting using the anti-EGFR (sc-71034), anti-PDGFR-a (sc-431), anti-PDGFR-B (sc-80291), and anti-Met (sc-8307) antibodies, all from Santa Cruz Biotechnologies Inc (Santa Cruz, Calif.). The appropriate horseradish peroxidase (HRP)-conjugated secondary antibody and Amersham ECL Plus Western Blotting Detection Reagent (GE Healthcare Bio-Sciences Corp, Piscataway, N.J.). Equivalent protein loading is confirmed by reprobing the membrane with anti-GAPDH (sc-47724) antibodies.

### Example 25

### Cell-Based Proliferation, Viability and Apoptosis Assays

The siRNA conjugates are transfected alone or with delivery vehicles into U87-MG glioblastoma cells (obtained from the American Type Culture Collection). In all cases, the conjugates are transfected at 50, 10, 2 and 0.4 nM final concentrations in triplicate. Control reactions include the individual (non-conjugated) siRNAs alone at the same concentrations, the combination of siRNAs (non-conjugated, but co-encapsulated in delivery vehicle), a negative control siRNA (Dharmacon), the drug alone (when applicable), and the delivery vehicle alone. At 48 and 72 hrs post-transfection, overall proliferation and viability is monitored using the CellTiter-Glo® Luminescent Cell Viability Assay following the manufacturer's recommended protocols (Promega, Madison, Wis.). The number of nonviable cells is similarly monitored using the CytoTox-ONE™ Homogeneous Membrane Integrity Assay (Promega). To characterize the apoptotic pathway(s) induced by the siRNA conjugates, lysates are also analyzed with the Caspase-Glo® 3/7, 8, and 9 Assays (Promega).

In addition, an in vitro soft agar assay is employed to quantify the effects of the siRNAs on the anchorage-independent growth modes of U87-MG cells. The soft agar assay is a routine test employed in cancer biology to examine anchorage independent growth modes of various cell lines. Anchorage independence correlates strongly with tumorigenicity and invasiveness in vivo. U87-MG cells are transfected as described above. Twenty-four hours post transfection, the cells are trypsinized and resuspended to 3000 cells/ml in 1 ml of growth media partially solidified with agarose (0.18% agarose/10% fetal bovine serum (FBS)/DMEM). The resuspended cells are layered onto a pre-formed agarose pad solidified in 6-well plates (each well contains 3 mls of DMEM/10% FBS supplemented with 20 mM Hepes (pH 7.5) and 0.25% agarose). Colonies>0.3 mm are visualized and counted on day 21 following staining with p-iodonitrotetrazolium violet (Sigma-Aldrich) (1 mg/ml).

### Example 26

### Activity of siRNA Conjugates In Vivo

NCr homozygous nude mice (6-8 weeks old) are purchased from Taconic (Germantown, N.Y.) at 6-8 weeks in age. In certain experiments, the U87-MG cells are transfected with the siRNA conjugates prior to injection in nude mice. One million U87-MG cells (resuspended in 100 μl of phosphate buffered saline (PBS), pH 7.4) are injected subcutaneously in the flanks of the nude mice. Tumor growth is monitored twice weekly for 10 weeks, and tumor volume (length×width×thickness) is measured using a Mitutoyo Digimatic caliper and micrometer. In other experiments, parental U87-MG cells are injected subcutaneously as described above. At two weeks post injection, the conjugates are directly injected into the same location (in 100 μl PBS, pH 7.4). The conjugates (naked or formulated with a delivery vehicle as described above) are injected once or twice per week, at a final dose of 1 mg/kg. Tumor growth is monitored twice weekly for an additional 10 weeks. This assay monitors the ability of the conjugates to inhibit tumor formation in vivo. In other experiments, parental U87-MG cells are injected subcutaneously as described above. At six weeks post injection, the conjugates (naked or formulated with a delivery vehicle as described above) are injected intratumorally twice per week, at a final dose of 1 mg/kg. Tumor growth is monitored twice weekly for an additional 6 weeks. This assay monitors the ability of the conjugates to regress established tumors in vivo. In all experiments, controls include injections with PBS alone, and with the individual agents (e.g. siRNA or drug) alone.

In addition to monitoring tumor volume and size, a subset of injected mice are sacrificed 48 and 96 hours following intratumoral injection of the siRNA conjugates. The tumors are harvested, macrodissected, and total RNA and protein extracts are prepared using the PARIS™ (Protein And RNA Isolation System) purification kit according to the manufacturer's instructions (Ambion, Austin, Tex.). siRNA activity is monitored at the RNA and protein levels by qRT-PCR and western blotting, respectively, as described in Example 24.

### Example 27

### Agent Selection for Glioblastoma Multiforme

Using the Collins mathematical approach, data can be collected on multiple glioblastoma multiforme samples from a range of tumor samples and compared to a compendium of samples containing diverse tissue types, normal human brain, normal human astrocytes, and normal human neurons. The following proteins may be identified as the most key effectors: EGFR, cyclin D1, MDM2, H-RAS, PDGFRA, CDK4, MDM4, K-RAS, PDGFRB, CDK6, Bcl-2, B-RAF, MET, c-Myc, Bcl-xl, MAPK, VEGFR1, MCL-1, p110α, VEGFR2, survivin, IRS1, IGFR1, Bcl2-L12, IRS2, IL-6, AKT-1, EGF, AKT-2, VEGF, AKT3, IGF, mTOR, HGF, Olig1, Olig2, Gli1, Gli2, Bmi1, Sox2, Oct3/4, Nanog, Fgf-4, Utf1, Lefty1, Stat3, c-Src, c-Yes, Fyn, Lck, Hck, Blk, c-Fgr, RON, AXL, AphA2, VEGFR3, and ROR2. Systematically, theoretical inhibitions of 50% and 100% of protein activity were explored for every combination of two, three, four, five, six, seven, eight, nine and ten proteins using the Genstruct causal modeling approach. Analysis may reveal that inhibition of AKT-1, EGFR, VEGFR, and K-RAS produces the greatest combination effect. To identify potent active agents, various siRNA designs can be tested for the ability to knock-down AKT1-, EGFR, VEGF, or K-RAS at 48 hours post-transfection in HeLa cells. Multiple designs may be determined to be effective, particularly those employing either the Dharmacon, Qiagen, Ambion, Invitrogen, or Integrated DNA Technologies (IDT) algorithms, some of which are based on commonly applied design rules in which siRNAs are complementary to 19-27 nt stretches within the target mRNA or 5′ or 3′ UTR, typically with a local GC content of 25-75%, minimal secondary structure, no identity to 19-27 nt stretches of other (non-target) human mRNAs. Some designs also eliminate candidate siRNA sequences with homology to the “seed” sequence of known human miRNAs and immune system recognition motifs to minimize activation of non-specific antiviral responses, including interferon gamma induction and activation of toll-like receptors. The most potent siRNA designs, conveying>80% knock-down of target mRNA within 48 hrs post-transfection at 1 nM concentration, can be selected as active agents. These siRNAs are then combined as described into a single RNAco-i entity. The RNAco-i formulation is transfected into the following glioblastoma cell lines U87-MG, LN18, A172, LN229 and U118 (American Type Culture Collection). Various concentrations, including 10 nM, may be found to reduce the viability and/or proliferative capacity of the cell lines in culture. The RNAco-i is then tested in both subcutaneous and orthotopic (intracranial) xenograft models, in which delivery is prior to, coincident with, or following injection of approximately one million GBM cells into the subcutaneous flanks or cranial cavities of nude mice. The RNAco-i formulations are expected to cause a significant reduction in tumor volume.

### Example 28

### Agent Selection for Psoriasis

Using the Institute for Systems Biology Method, data can be collected on multiple psoriasis samples from a range of tissues naturally affected. Specifically, proteomic analysis can be performed on dermis, keratinocytes, T cells and NK cells, with a screen for overexpressed genes. The following proteins may be identified as key targets for knockdown: IL-1, IL-6, IL-12, IL-17, IL-19, IL-20, IL-22, IL-23, IL-22R, IFN-α, IFN-γ, Stat1, Stat3, NF-κB, TGF-β, TNF-α, TGF-a, IGF-1, NGF, amphiregulin, ECGF, VEGF, PDGF, KGF, Tie2, MMP9, and TNF-β (lymphotoxin (LT)). Systematically, theoretical inhibitions of 50% and 100% of protein activity are explored for every combination of two, three, four, five, six, seven, eight, nine and ten proteins suing the Genstruct causal modeling approach. Analysis may reveal that inhibition of VEGF, TGF-β, TNF-α, IFN-α, IL-23 and IL-1 produces the greatest combination effect.

To identify potent active agents, various siRNA designs can be tested for the ability to knock-down VEGF, TGF-β, TNF-α, IFN-α, IL-23 and IL-1 at 48 hours post-transfection in HeLa or Jurkat T-cells. Multiple designs may be determined to be effective, particularly those employing either the Dharmacon, Qiagen, Ambion, Invitrogen, or Integrated DNA Technologies (IDT) algorithms, some of which are based on commonly applied design rules in which siRNAs are complementary to 19-27 nt stretches within the target mRNA or 5′ or 3′ UTR, typically with a local GC content of 25-75%, minimal secondary structure, no identity to 19-27 nt stretches of other (non-target) human mRNAs. Some designs also eliminate candidate siRNA sequences with homology to the “seed” sequence of known human miRNAs and immune system recognition motifs to minimize activation of non-specific antiviral responses, including interferon gamma induction and activation of toll-like receptors. The most potent siRNA designs, conveying>80% knock-down of target mRNA within 48 hrs post-transfection at 1 nM concentration, are then selected as active agents. These siRNAs are then combined as described into a single RNAco-i entity. Empirical testing can be performed on keratinocyte cultures isolated from affected psoriatic and non-affected biopsies by serial disaggregation using enzymatic and mechanical techniques as described (Stan C et al. “Cellular and molecular changes of psoriatic keratinocytes in response to UVA in vitro treatment.” Romanian J Biophys (2004); 14(1-4): 1-12). Administration of the RNAco-i may have minimal effects on the proliferation of normal or psoriatic keratinocytes cell cultures in vitro, which is not surprising given that this model fails to mimic the complex pathogenic milieu observed in actual psoriatic plaques, with significant immune infiltration and angiogenic components. Therefore, the RNAco-i was administered topically to psoriatic lesions of SCID (Nickoloff B J et al. “Severe combined immunodeficiency mouse and human psoriatic skin chimeras.” Am J Pathol (1995), 146:580-88) and AGR129 mouse xenograft models (Boyman O et al. “Spontaneous development of psoriasis in new animal model shows an essential role for resident T-cells and tumor necrosis factor-α.” J Exp Med (2004), 199(5):731-6). The RNAco-i can be expected to show a significant amelioration of the psoriatic phenotype, including a reduction in scaling and reduced reddening.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

We claim:

1. An RNA co-interference composition comprising:
(a) a first region of contiguous ribonucleotides defining a first double-stranded oligoribonucleotide complementary to a hybridization sequence of a target nucleic acid, said first oligoribonucleotide having at least one functional group;
(b) a second region of contiguous ribonucleotides defining a second double-stranded oligoribonucleotide complementary to a hybridization sequence of said target nucleic acid, said second oligoribonucleotide having at least one functional group; and
(c) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide;
wherein said first oligoribonucleotide and said second oligoribonucleotide are joined by said linking moiety through said functional groups of said first oligoribonucleotide and said second oligoribonucleotide with said reactive groups of said linking moiety, and
wherein said RNA co-interference composition is capable of modulating expression of said target nucleic acid through RNA co-interference.

2. An RNA co-interference composition comprising:
(a) a first region of contiguous ribonucleotides defining a first double-stranded oligoribonucleotide complementary to a hybridization sequence of a first target nucleic acid, said first oligoribonucleotide having at least one functional group;
(b) a second region of contiguous ribonucleotides defining a second double-stranded oligoribonucleotide complementary to a hybridization sequence of a second target nucleic acid, said second oligoribonucleotide having at least one functional group; and
(c) a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; wherein said first oligoribonucleotide and said second oligoribonucleotide are joined by said linking moiety through said functional groups of said first oligoribonucleotide and said second oligoribonucleotide with said reactive groups of said linking moiety, and wherein said RNA co-interference composition is capable of modulating expression of said first target nucleic acid and said second target nucleic acid through RNA co-interference.

3. An RNA co-interference composition comprising:
(a) a first region of contiguous ribonucleotides defining a first double-stranded oligoribonucleotide complementary to a first hybridization sequence of a first target nucleic acid, said first oligoribonucleotide having at least one functional group;
(b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group; (c) a third region of contiguous ribonucleotides defining a third double-stranded oligoribonucleotide complementary to a hybridization sequence of any of the foregoing target nucleic acids, or to a hybridization sequence of a different target nucleic acid said third oligoribonucleotide having at least one functional group; (d) is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; wherein said first oligoribonucleotide, said second oligoribonucleotide, and said third oligoribonucleotide are joined by said linking moiety through said functional groups of said first oligoribonucleotide, said second oligoribonucleotide, and said third oligoribonucleotide with said reactive groups of said linking moiety, and wherein said RNA co-interference composition is capable of modulating expression of said target nucleic acid through RNA co-interference.

4. An RNA co-interference composition comprising:
(a) a first region of contiguous ribonucleotides defining a first double-stranded oligoribonucleotide complementary to a first hybridization sequence of a first target nucleic acid, said first oligoribonucleotide having at least one functional group;
(b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group;
(c) a third region of contiguous ribonucleotides defining a third double-stranded oligoribonucleotide complementary to a hybridization sequence of any of the foregoing target nucleic acids, or to a hybridization sequence of a different target nucleic acid said third oligoribonucleotide having at least one functional group;
(d) is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and
(e) a second linking moiety having at least two reactive groups reactive with two or more functional groups corresponding and reactive with said second linking moiety, wherein said first oligoribonucleotide, said second oligoribonucleotide, and said third oligoribonucleotide are joined by said first linking moiety and said second linking moiety through said functional groups of said first oligoribonucleotide, said second oligoribonucleotide, and said third oligoribonucleotide with said reactive groups of said first linking moiety and said second linking moiety, and
wherein said RNA co-interference composition is capable of modulating expression of said target nucleic acids to which said first oligoribonucleotide, said second oligoribonucleotide, and said third oligoribonucleotide are complementary through RNA co-interference.

5. A RNA co-interference composition comprising:
(a) a first region of contiguous ribonucleotides defining a first double-stranded oligoribonucleotide complementary to a hybridization sequence of a first target nucleic acid, said first oligoribonucleotide having at least one functional group;
(b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group;
(c) a third region of contiguous ribonucleotides defining a third double-stranded oligoribonucleotide complementary to a hybridization sequence of any of the foregoing target nucleic acids, or to a hybridization sequence of a different target nucleic acid;
(d) a fourth region of contiguous ribonucleotides defining a fourth double-stranded oligoribonucleotide complementary to hybridization sequence of any of the foregoing target nucleic acids, or to a hybridization sequence of a different target nucleic acid, said fourth oligoribonucleotide having at least one functional group;
(d) one or more linking moieties that are the same or different comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide;
wherein said first oligoribonucleotide, said second oligoribonucleotide, said third oligoribonucleotide and said fourth oligoribonucleotide are joined by said linking moiety through said functional groups of said first oligoribonucleotide, said second oligoribonucleotide, said third oligoribonucleotide and said fourth oligoribonucleotide with said reactive groups of said linking moiety, and
wherein said RNA co-interference composition is capable of modulating expression of said target nucleic acids to which said first oligoribonucleotide, said second oligoribonucleotide, said third oligoribonucleotide and said fourth oligoribonucleotide are complementary through RNA co-interference.

6. A RNA co-interference composition comprising:
(a) a first region of contiguous ribonucleotides defining a first double-stranded oligoribonucleotide complementary to a hybridization sequence of a first target nucleic acid, said first oligoribonucleotide having at least one functional group;
(b) a second region of contiguous ribonucleotides defining a second double-stranded oligoribonucleotide complementary to a hybridization sequence of the foregoing target nucleic acid, or to a hybridization sequence of a different target nucleic acid, said second oligoribonucleotide having at least one functional group;
(c) a third region of contiguous ribonucleotides defining a third double-stranded oligoribonucleotide complementary to a hybridization sequence of any of the foregoing target nucleic acids, or to a hybridization sequence of a different target nucleic acid;
(d) a fourth region of contiguous ribonucleotides defining a fourth double-stranded oligoribonucleotide complementary to a hybridization sequence of any of the foregoing target nucleic acids, or to a hybridization sequence of a different target nucleic acid, said fourth oligoribonucleotide having at least one functional group;
(d) is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and
(e) one or more additional linking moieties that are the same or different having at least two reactive groups reactive with two or more functional groups corresponding and reactive with said additional linking moieties,
wherein said first oligoribonucleotide, said second oligoribonucleotide, said third oligoribonucleotide and said fourth oligoribonucleotide are joined by said first linking moiety and said additional linking moieties through said functional groups of said first oligoribonucleotide, said second oligoribonucleotide, said third oligoribonucleotide and said fourth oligoribonucleotide with said reactive groups of said first linking moiety and said additional linking moieties, and
wherein said RNA co-interference composition is capable of modulating expression of said target nucleic acids to which said first oligoribonucleotide, said second oligoribonucleotide, said third oligoribonucleotide and said fourth oligoribonucleotide are complementary through RNA co-interference.

7. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference and wherein the sense strand or anti-sense strand is modified by the substitution of at least one internal ribonucleotide with a modified ribonucleotide.

8. The composition of any one of claims 2-4, wherein the hydrophilic polymer is a co-block polymer.

9. The RNA co-interference composition claim 7, further comprising an additional conjugating linker for linking a conjugate moiety to at least one of the oligoribonucleotides.

10. The RNA co-interference composition of claim 9, wherein the conjugate moiety is selected from the group consisting of: a sugar, a polysaccharide, a lipid, RNA, DNA, aromatic and non-aromatic lipophilic molecules including steroid molecules, proteins including antibodies, enzymes, and serum proteins, peptides, water-soluble and lipidsoluble vitamins, water-soluble and lipid-soluble polymers, small molecules including drugs, toxins, reporter molecules, and receptor ligands, a metabolite, carbohydrate complexes, nucleic acid cleaving complexes, metal chelators including porphyrins, texaphyrins, and crown ethers, intercalators including hybrid photonucleaselintercalators and photoactive and redox active crosslinking agents.

11. The RNA co-interference composition of claim 7, wherein said RNA co-interference composition has enhanced in vivo stability as compared to the corresponding unmodified oligoribonucleotides.

12. The RNA co-interference composition of claim 7, wherein said RNA co-interference composition has enhanced target efficacy as compared to the corresponding unmodified oligoribonucleotides.

13. The RNA co-interference composition of claim 7, wherein said RNA co-interference composition has enhanced cellular penetration as compared to the corresponding unmodified oligoribonucleotides.

14. The RNA co-interference composition of claim 7, wherein at least one of the modified ribonucleotides is a sugar-modified ribonucleotide.

15. The RNA co-interference composition of claim 7, wherein at least one of the modified ribonucleotides is a nucleobase-modified ribonucleotide.

16. The RNA co-interference composition of claim 7, wherein a target sequence specifies an amino acid sequence of a cellular protein.

17. The RNA co-interference composition of claim 7, wherein a target sequence specifies an amino acid sequence of a viral protein.

18. The RNA co-interference composition of claim 7, wherein the modified ribonucleotide is selected from the group consisting of 2′-deoxy ribonucleotide, 2′-fluoro ribonucleotide, 2′-deoxy-2′-fluoro, 2′-amino ribonucleotide, 2′-O-methyl ribonucleotide, 2′-O-(2-methoxyethyl), and 2′-thio ribonucleotide.

19. The RNA co-interference composition of claim 7, wherein the modified ribonucleotide is a 2′-deoxy ribonucleotide.

20. The RNA co-interference composition of claim 18, wherein the modified ribonucleotide is in the sense strand.

21. The RNA co-interference composition of claim 17, wherein the modified ribonucleotide is in the anti-sense strand.

22. The RNA co-interference composition of claim 17, wherein the modified ribonucleotides are in the sense and anti-sense strands.

23. The RNA co-interference composition of claim 7, wherein the modified ribonucleotide is selected from the group consisting of 2′-fluoro cytidine, 2′-fluoro-uridine, 2′-fluoro adenosine, 2-fluoro guanosine, 2′-amino cytidine, 2′-amino adenosine, 1′ amino guanosine and 2′-amino-butyryl-pyrene uridine.

24. The RNA co-interference composition of claim 7, wherein the modified ribonucleotide is selected from the group consisting of 5-bromo-uridine, 5-iodo-uridine, 5-methyl-cytidine, ribo-thymidine, 2-aminoopurine, 4-thio-uridine and 5-amino-allyl-uridine.

25. The RNA co-interference composition of claim 7, wherein the modified ribonucleotide is a back-bone modified ribonucleotide.

26. The RNA co-interference composition of claim 7, wherein the modified ribonucleotide contains a phosphorothiolate group.

27. The RNA co-interference composition of claim 7, wherein a 3′-OH terminus of the sense strand or anti-sense strand is modified.

28. The RNA co-interference composition of any one of claims 2-4, wherein the oligonucleotides are between about 10 to 50 residues in length.

29. The RNA co-interference composition of claim 28, wherein the oligonucleotides are between about 15 to 45 residues in length.

30. The RNA co-interference composition of claim 28, wherein the oligonucleotides are between about 20 to 40 residues in length.

31. The RNA co-interference composition of claim 28, wherein the oligonucleotides are between about 19 to 25 residues in length.

32. The RNA co-interference composition of claim 28, wherein the oligonucleotides are between about 19 to 22 residues in length.

33. The RNA co-interference composition of claim 28, wherein the oligonucleotides are between about 21 to 22 residues in length.

34. The RNA co-interference composition of claim 28, wherein the oligonucleotides are between about 27 to 29 residues in length.

35. The RNA co-interference composition of any one of claims 2-4, wherein the oligonucleotides are chemically synthesized.

36. The RNA co-interference composition of claim 28, wherein the composition has a net positive charge.

37. The RNA co-interference composition of claim 28, wherein the composition has a positive zeta potential.

38. A composition comprising the RNA co-interference composition of any one of claims 2-4 and a pharmaceutically acceptable carrier.

39. A method of activating target-specific RNA co-interference in a cell comprising introducing into said cell the RNA co-interference composition of any one of the preceding claims, said RNA co-interference composition being introduced in an amount sufficient for modulation of a target nucleic acid to occur, thereby activating target specific RNA co-interference in the cell.

40. A method of claim 39, wherein the RNA co-interference composition is introduced into the cell by contacting the cell with the RNA co-interference composition.

41. A method of claim 39, wherein the RNA co-interference composition is introduced into the cell by contacting the cell with a composition comprising the RNA co-interference composition and a lipophilic carrier.

42. The method of any of claims 39-41, wherein the target nucleic acid specifies the amino acid sequence of a protein involved in or predicted to be involved in a human disease, disorder, condition or trait.

43. A method of activating a target-specific RNA co-interference in an organism comprising administering to said organism the RNA co-interference composition of any one of claims 2-4, said RNA co-interference composition being administered in an amount sufficient for modulation of a target nucleic acid to occur, thereby activating target specific RNA co-interference in the organism.

44. The method of claim 39, wherein the RNA co-interference composition is administered by an intravenous, intramuscular, subcutaneous, or intraperitoneal injection, topical application, local infusion, or oral administration.

45. The method of claim 39, wherein the target nuclei acid specifies the amino acid sequence of a protein involved in or predicted to be involved in a human disease, disorder, condition or trait.

46. The method of claims 39 or 43, wherein modulation of the target nucleic acid produces a loss-of-function phenotype.

47. The method of claim 39 or 43, wherein modulation of the target nucleic acid sequence corresponds to a decrease of at least 10 percent of the protein specified by said target nucleic acid.

48. The method of claim 39 or 43, wherein modulation of the target nucleic acid sequence corresponds to a decrease of at least 25 percent of the protein specified by said target nucleic acid.

49. The method of claim 39 or 43, wherein modulation of the target nucleic acid sequence corresponds to a decrease of at least 50 percent of the protein specified by said target nucleic acid.

50. The method of claim 39 or 43, wherein modulation of the target nucleic acid sequence corresponds to a decrease of at least 75 percent of the protein specified by said target nucleic acid.

51. The method of claim 39 or 43, wherein modulation of the target nucleic acid sequence corresponds to a decrease of at least 90 percent of the protein specified by said target nucleic acid.

52. A method of evaluating gene function in a cell or an organism, comprising:
(a) introducing into said cell or organism an RNA co-interference composition;
(b) maintaining the cell or organism under conditions allowing target-specific RNA co-interference to occur;
(c) determining a characteristic or property of said cell or said organism; and
(d) comparing said characteristic or property to a suitable control, the comparison yielding information about the function of the gene.

53. A method of validating a candidate protein as a suitable target for drug discovery, comprising:
(a) introducing into a cell or organism an RNA co-interference composition;
(b) maintaining the cell or organism under conditions allowing target-specific RNA co-interference to occur;
(c) determining a characteristic or property of said cell or said organism; and
(d) comparing said characteristic or property to a suitable control, the comparison yielding information about whether the candidate protein is a suitable target for drug discovery.

54. A method of validating a candidate RNA co-interference composition as a suitable composition for drug therapy, comprising:
(a) introducing into a cell or organism an RNA co-interference composition;
(b) maintaining the cell or organism under conditions allowing target-specific RNA co-interference to occur;
(c) determining a characteristic or property of said cell or said organism; and
(d) comparing said characteristic or property to a suitable control, the comparison yielding information about whether the candidate RNA co-interference composition is a suitable target for drug therapy.

55. A kit comprising reagents for activating target-specific RNA co-interference in a cell or organism, said kit comprising:
(a) one or more RNA co-interference compositions; and
(b) instructions for use.

56. A method of treating a disease, disorder, condition or trait associated with the activity of a protein specified by a target nucleic acid in a subject, comprising administering to said subject the RNA co-interference composition of any of claims 2-4, said RNA co-interference composition being administered in an amount sufficient for modulation of the target nucleic acid to occur, thereby treating the disease, disorder, condition or trait associated with the protein.

57. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 80% sequence identity of said anti-sense strand and said target nucleic acid.

58. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 90% sequence identity of said anti-sense strand and said target nucleic acid.

59. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 91% sequence identity of said anti-sense strand and said target nucleic acid.

60. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 92% sequence identity of said anti-sense strand and said target nucleic acid.

61. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 93% sequence identity of said anti-sense strand and said target nucleic acid.

62. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 94% sequence identity of said anti-sense strand and said target nucleic acid.

63. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 95% sequence identity of said anti-sense strand and said target nucleic acid.

64. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 96% sequence identity of said anti-sense strand and said target nucleic acid.

65. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 97% sequence identity of said anti-sense strand and said target nucleic acid.

66. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 98% sequence identity of said anti-sense strand and said target nucleic acid.

67. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 99% sequence identity of said anti-sense strand and said target nucleic acid.

68. The RNA co-interference composition of any one of claims 2-4, wherein the oligoribonucleotides of said RNA co-interference composition comprise a sense and anti-sense strand, wherein the anti-sense strand has a sequence sufficiently complementary to a target nucleic acid sequence to direct target specific RNA co-interference, wherein sufficient complementarity is achieved by about 100% sequence identity of said anti-sense strand and said target nucleic acid.

69. The RNA co-interference composition of any one of claims 2-4, wherein the composition comprises a fixed ratio of double-stranded oligoribonucleotides.

70. The RNA co-interference composition of any of one of claims 2-4, wherein the composition synergistically modulates expression of one or more target nucleic acids through RNA co-interference.

71. An RNA co-interference composition having the structural formula:
A-L1-L2-B, wherein
(a) A is a double-stranded oligoribonucleotide complementary to a first hybridization sequence of a first target nucleic acid;
(b) L1 is a first linking moiety comprising a non-biologically active strand of RNA or DNA capable of being cleaved endogenously, thereby releasing said oligoribonucleotide from said first linking moiety L1, said first linking moiety having at least one functional group;
(c) L2 is a second linking moiety capable of covalently bonding to linker L1 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and
(d) B is a second double-stranded oligoribonucleotide complementary to a hybridization sequence of one or more target nucleic acids, wherein said hybridization sequence is (i) the same or different to said first hybridization sequence of said first target nucleic acid; or (ii) a hybridization sequence to a second target nucleic acid, said second oligoribonucleotide having at least one functional group,
wherein said second oligoribonucleotide is capable of being joined to said second linking moiety L2 through interaction of said functional group and said reactive group; and
wherein said RNA co-interference composition is capable of modulating expression of said one or more target nucleic acids through RNA co-interference.

72. An RNA co-interference composition having the structural formula:
A-L-B, wherein
(a) A is a double-stranded oligoribonucleotide complementary to a first hybridization sequence of a first target nucleic acid, said first oligoribonucleotide having at least one functional group;
(b) L is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and
(c) B is a second double-stranded oligoribonucleotide complementary to a hybridization sequence of a second target nucleic acids, wherein said hybridization sequence is (i) the same or different to said first hybridization sequence of said first target nucleic acid; or (ii) a hybridization sequence to a second target nucleic acid, said second oligoribonucleotide having at least one functional group,
wherein said first and said second oligoribonucleotides are capable of being joined by said linking moiety L through interaction of said functional groups and said reactive groups; and
wherein said RNA co-interference composition is capable of modulating expression of said first or said first and said second target nucleic acids through RNA co-interference.

73. An RNA co-interference composition having the structural formula:
A-L1-L2-X, wherein
(a) A is a double-stranded oligoribonucleotide complementary to a first hybridization sequence of a first target nucleic acid;
(b) L1 is a first linking moiety comprising a non-biologically active strand of RNA or DNA capable of being cleaved endogenously, thereby releasing said oligoribonucleotide from said first linking moiety, said first linking moiety having at least one functional group;
(c) L2 is a second linking moiety capable of bonding to linker L1 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide, and having one or more one reactive groups; and
(d) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups,
wherein said one or more oligoribonucleotides is capable of being joined to said second linking moiety L2 through interaction of said one or more functional groups and said one or more reactive groups; and
wherein said RNA co-interference composition is capable of modulating expression of said one or more target nucleic acids through RNA co-interference.

74. An RNA co-interference composition having the structural formula:
A-L-X, wherein
(a) A is a double-stranded oligoribonucleotide complementary to a first hybridization sequence of a first target nucleic acid;
(b) L is a linking moiety capable of covalently bonding to two or more oligoribonucleotides, comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and
(c) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups,
wherein said one or more oligoribonucleotides is capable of being joined to said linking moiety L through interaction of said one or more functional groups and said one or more reactive groups; and
wherein said RNA co-interference composition is capable of modulating expression of said one or more target nucleic acids through RNA co-interference.

75. An RNA co-interference polymeric composition having the structural formula:
A-[L-X]ₙ, wherein
(a) A is a double-stranded oligoribonucleotide complementary to a first hybridization sequence of a first target nucleic acid;
(b) L is a linking moiety capable of covalently bonding to two or more oligoribonucleotides, comprising the same or different branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide; and
(c) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups,
wherein n is the integer 1 to about 500, and
wherein said one or more oligoribonucleotides X are capable of being joined to said linking moiety L through interaction of said one or more functional groups and said one or more reactive groups;
wherein said joined linking moiety L and said one or more oligoribonucleotides X comprise repeating branched or unbranched monomeric units of said RNA co-interference polymeric composition
wherein said RNA co-interference composition is capable of modulating expression of said one or more target nucleic acids through RNA co-interference.

76. An RNA co-interference composition having the structural formula:
A-L1-L2-[L3-X]n, wherein
(a) A is a double-stranded oligoribonucleotide complementary to a first hybridization sequence of a first target nucleic acid;
(b) L1 is a first linking moiety comprising a non-biologically active strand of RNA or DNA capable of being cleaved endogenously, thereby releasing said oligoribonucleotide A from said first linking moiety L1, said first linking moiety having at least one functional group;
(c) L2 is a second linking moiety capable of bonding linkers L1 and L3 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide, and having one or more one reactive groups;
(d) L3 is a third linking moiety the same or different to said first linking moiety L1, wherein said third linking moiety comprises a non-biologically active strand of RNA or DNA capable of being cleaved endogenously, thereby releasing said one or more oligoribonucleotide X from said third linking moiety L3, said third linking moiety having at least one functional group; and
(e) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups,
wherein n is the integer 1 to about 500, and
wherein said one or more oligoribonucleotides X are capable of being joined to said second linking moiety L2 through interaction of said one or more functional groups and said one or more reactive groups;
wherein said RNA co-interference composition is capable of modulating expression of said one or more target nucleic acids through RNA co-interference.

77. An RNA co-interference composition having the structural formula:
A-L1-L2-[L3n₁-X]n₂, wherein
(a) A is a double-stranded oligoribonucleotide complementary to a first hybridization sequence of a first target nucleic acid;
(b) L1 is a first linking moiety comprising a non-biologically active strand of RNA or DNA capable of being cleaved endogenously, thereby releasing said oligoribonucleotide A from said first linking moiety L1, said first linking moiety having at least one functional group;
(c) L2 is a second linking moiety capable of bonding linkers L1 and L3 comprising a branched or unbranched hydrophilic polymer selected from the group consisting of: polyamino acids, amino sugars, fatty acyl, glycerolipid, glycerophospholipid, sphinglipid, sterol lipid, prenol lipid, saccarolipid, polyketide, glucosamines, lipopolysaccarides, aminopolysaccarides, polyglutamic acids, poly(allylamines), polyethylene glycol (PEG), PEG derivatives, methoxy polyethylene glycol (mPEG), polypropylene glycol (PPG), poly(lactic acid), poly(glycolic acid), poly(ethylene-co-vinyl acetate) (EVAc), N-(2-hydroxypropyl)methacrylamides (HPMA), HPMA derivatives, poly(hydroxyalkanoates), poly(2-dimethylamino)ethyl methacrylate (DMAEMA), poly(D,L lactic-co-glycolide) (PLGA), poly(lactic-co-glycolic acid) (PLGA), PLGA derivatives, poly(polypropylacrylic acid) (PPAA), poly(D,L-lactide)-block-methoxypolyethylene glycol (diblock), poly(ethyleneimine), poly(beta-aminoester), polyvinyl alcohol, poly(hydroxyethyl methacrylate), polyacrylamide, polyacrylic acid, polyethyloxazole, polyvinyl pyrrolidinone, and polysaccharides such as dextran, chitosan, alginates, hyaluronic acid, and any ratio of copolymers, grafted polymers, and grafted copolymers thereof, wherein said linking moiety further comprises at least two terminal reactive groups corresponding and reactive with two or more functional groups selected from the group consisting of: OH, —COOH, N-hydroxy succidimidyl ester (NHS), Imidazole amide, triazole amide, tetrazole amide, hydroxy benzotriazole ester (HOBt), 1-hydroxy-7-azabenzotriazole ester (HOAt), 2,4-dinitrophenyl ester, pentafluorophenyl ester, 2,2,2-trifluoroethyl ester, 2,2,2-trifluoroethyl thioester, acid chloride, acid bromide, 4-nitrophenyl carbonate (NPC), isocyanate, optionally substituted aldehyde, optionally substituted ketone, optionally substituted acrylate, maleimide, vinyl sulfone, and orthopyridyl disulfide, and having one or more one reactive groups; and
(d) L3 is a third linking moiety the same or different to said first linking moiety L1, wherein said third linking moiety comprises a non-biologically active strand of RNA or DNA capable of being cleaved endogenously, thereby releasing said one or more oligoribonucleotide X from said third linking moiety L3, said third linking moiety having at least one functional group;
(e) X is one or more double-stranded oligoribonucleotides complementary to one or more hybridization sequences of one or more target nucleic acids, wherein said one or more hybridization sequences are the same or different to said first hybridization sequence of said first target nucleic acid, said one or more double-stranded oligoribonucleotide having at least one or more functional groups,
wherein n₁ is the integer 0 or 1,
wherein n₂ is the integer 1 to about 500,
wherein X may optionally contain a reactive group when n₁=0,
wherein such oligoribonucleotide X having a reactive group is capable of being joined to another oligoribonucleotide X having one or more functional groups through interaction of said reactive group with said one or more functional groups of said another oligoribonucleotide X,
wherein said one or more oligoribonucleotides X are capable of being joined to said second linking moiety L2 through interaction of said one or more functional groups and said one or more reactive groups;
wherein said RNA co-interference composition is capable of modulating expression of said one or more target nucleic acids through RNA co-interference.

78. The RNA co-interference composition of any of claims 73 to 75, wherein the oligoribonucleotides of said RNA co-interference composition comprise a sense and an anti-sense strand, wherein the anti-sense strand has a sequence sufficiently complementary to a target nucleic acid to direct target specific RNA co-interference and wherein the sense strand or the anti-sense strand is modified by substitution of at least one internal ribonucleotide with a modified ribonucleotide.

79. The RNA co-interference composition of any of claims 73 to 75, further comprising an additional conjugating linker for linking a conjugate moiety to at least one of the oligoribonucleotides.

80. The RNA co-interference composition of claim 79, wherein the conjugate moiety is selected from the group consisting of: a sugar, a polysaccharide, a lipid, RNA, DNA, aromatic and non-aromatic lipophilic molecules including steroid molecules, proteins including antibodies, enzymes, and serum proteins, peptides, water-soluble and lipidsoluble vitamins, water-soluble and lipid-soluble polymers, small molecules including drugs, toxins, reporter molecules, and receptor ligands, a metabolite, carbohydrate complexes, nucleic acid cleaving complexes, metal chelators including porphyrins, texaphyrins, and crown ethers, intercalators including hybrid photonucleaselintercalators and photoactive and redox active crosslinking agents.

81. The RNA co-interference composition of any of claims 73 to 75, wherein at least one of the double-stranded oligoribonucleotide is selected from the group consisting of siRNA, microRNA, and short hairpin RNA, and mixtures of (a), (b) and (c)

82. The RNA co-interference composition of any of claims 73 to 75, wherein each double-stranded oligoribonucleotide is a microRNA.

83. The RNA co-interference composition of any of claims 73 to 75, wherein each double-stranded oligoribonucleotide is a siRNA.

84. The RNA co-interference composition of any of claims 73 to 75, wherein each double-stranded oligoribonucleotide is a short hairpin RNA.

85. The RNA co-interference composition of claim 78, wherein at least one of the modified ribonucleotides is a sugar-modified ribonucleotide.

86. The RNA co-interference composition of claim 78, wherein at least one of the modified ribonucleotides is a nucleobase-modified ribonucleotide.

87. The RNA co-interference composition of claim 78, wherein at least one of the modified ribonucleotides is in the sense strand.

88. The RNA co-interference composition of claim 78, wherein at least one of the modified ribonucleotides is in the anti-sense strand.

89. The RNA co-interference composition of claim 78, wherein the modified ribonucleotides are in the sense and anti-sense strands.

90. The RNA co-interference composition of claim 78, wherein at least one of the modified ribonucleotides is a back-bone modified ribonucleotide.

91. The RNA co-interference composition of claim 78, wherein at least one of the modified ribonucleotides contains a phosphorothiolate group.

92. The RNA co-interference composition of claim 78, wherein a 3′-OH terminus of the sense strand or anti-sense strand of at least one of the oligoribonucleotides is modified.

93. The RNA co-interference composition of claim 78, wherein at least one of the modified ribonucleotides is a 2′-deoxy modified ribonucleotide.

94. The RNA co-interference composition of any one of claims 73 to 75, wherein the oligonucleotides are between about 10 to 50 residues in length.

95. The RNA co-interference composition of any one of claims 73 to 75, wherein the oligonucleotides are between about 15 to 45 residues in length.

96. The RNA co-interference composition of any one of claims 73 to 75, wherein the oligonucleotides are between about 20 to 40 residues in length.

97. The RNA co-interference composition of any one of claims 73 to 75, wherein the oligonucleotides are between about 19 to 25 residues in length.

98. The RNA co-interference composition of any one of claims 73 to 75, wherein the oligonucleotides are between about 19 to 22 residues in length.

99. The RNA co-interference composition of any one of claims 73 to 75, wherein the oligonucleotides are between about 21 to 22 residues in length.

100. The RNA co-interference composition of any one of claims 73 to 75, wherein the oligonucleotides are between about 25 to 27 residues in length.

101. The RNA co-interference composition of any one of claims 73 to 75, wherein the oligonucleotides are chemically synthesized.

102. A composition comprising the RNA co-interference composition of any one of claims 73 to 75 and a pharmaceutically acceptable carrier.

103. The RNA co-interference composition of any one of claims 2 to 4 or 73 to 75, wherein the composition is capable of synergistically modulating expression of said one or more target nucleic acids through RNA co-interference.

104. The composition of any one of claims 73 to 75, wherein the hydrophilic polymer is a co-block polymer.

105. The RNA co-interference composition of any one of claims 73 to 75, wherein the composition comprises a fixed ratio of double-stranded oligoribonucleotides.

106. The RNA co-interference composition of any one of claims 71, 73, or 76, wherein said first linking moiety comprises single-stranded RNA or single-stranded DNA.

107. The RNA co-interference composition of any one of claims 71, 73, or 76, wherein said first linking moiety comprises double-stranded RNA or double-stranded DNA.

108. The RNA co-interference composition of any one of claims 71, 73, or 76, wherein said first linking moiety comprises partially single stranded RNA or partially single stranded DNA.

109. The RNA co-interference composition of claim 76, wherein said third linking moiety is selected from the group consisting of single-stranded RNA, single-stranded DNA, double-stranded RNA, double-stranded DNA, partially single stranded RNA and partially single stranded DNA.

110. The RNA co-interference composition of any one of claim 75 to 77, wherein the integer n=1 to about 250.

111. The RNA co-interference composition of any one of claim 75 or 77, wherein the integer n=1 to about 125.

112. The RNA co-interference composition of any one of claim 75 or 76, wherein the integer n=1 to about 100.

113. The RNA co-interference composition of any one of claim 75 or 76, wherein the integer n=1 to about 12

114. The RNA co-interference composition of any one of claim 75 or 76, wherein the integer n=1 to about 10

115. The RNA co-interference composition of any one of claim 75 or 76, wherein the integer n=1 to about 8

116. The RNA co-interference composition of any one of claim 75 or 76, wherein the integer n=1 to about 6

117. The RNA co-interference composition of any one of claim 75 or 76, wherein the integer n=1 to about 5

118. The RNA co-interference composition of any one of claim 75 or 76, wherein the integer n=1 to about 4.

119. The RNA co-interference composition of any one of claim 75 or 76, wherein the integer n=1 to about 3.

120. The RNA co-interference composition of any one of claim 75 or 76, wherein the integer n=1 to 2.

121. The RNA co-interference composition of any one of claim 75 or 76, wherein the integer n=1.

122. A method of evaluating gene function in a cell, population of cells, or an organism, comprising:
(a) introducing into said cell, said population of cells or said organism one or more RNA co-interference compositions of any one of claims 2 to 4 or 73 to 75;
(b) maintaining said cell, said population of cells or said organism under conditions allowing target-specific RNA co-interference to occur;
(c) determining a characteristic, property or phenotype of said cell, said population of cells or said organism; and
(d) comparing said characteristic, property or phenotype to a suitable control, the comparison yielding information about the function of the gene.

123. The method of claim 122, wherein the oligoribonucleotides of the RNA co-interference composition are selected in a manner capable of predicting synergistic modulation of said gene function.

124. A method of validating a candidate protein as a suitable target for drug discovery, comprising:
(a) introducing into a cell, a population of cells or an organism one or more RNA co-interference composition of any one of claims 2 to 4 or 73 to 75;
(b) maintaining said cell, said population of cells or said organism under conditions allowing target-specific RNA co-interference to occur;
(c) determining a characteristic, property or phenotype of said cell, said population of cells or said organism; and
(d) comparing said characteristic, property or phenotype to a suitable control, the comparison yielding information about whether the candidate protein is a suitable target for drug discovery.

125. A method of designing a RNA co-interference composition of any one of claims 2 to 4 or 73 to 75 comprising:
(a) specifying a phenotype of interest associated with a disease, disorder, condition or trait affecting a cell, population of cells or organism;
(b) specifying a biochemical network of such cell, such population of cells or such organism postulated to be correlated to said specified phenotype;
(c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations;
(d) optimizing said simulated biochemical network by determining and constraining the values of said quantitative parameters of said interrelationships;
(e) solving said mathematical equations to identify interrelationships likely to cause the transition of said cell, said population of cells or said organism from said phenotype to another phenotype, thereby identifying one or more nucleic acids associated with said phenotypic change;
(f) preparing two or more double-stranded oligoribonucleotides complementary to said nucleic acids identified in step (e) capable of modulating expression of said nucleic acids through RNA co-interference; and
(g) preparing an RNA co-interference composition comprising said nucleic acids prepared in accordance with step (f).

126. A method of designing a RNA co-interference composition of any one of claims 2 to 4 or 73 to 75, comprising:
(a) specifying a phenotype of interest associated with a disease, disorder, condition or trait affecting a cell, population of cells or organism;
(b) specifying a biochemical network of such cell, such population of cells or such organism postulated to be correlated to such specified phenotype;
(c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations;
(d) solving said mathematical equations to identify interrelationships likely to cause the transition of said cell, said population of cells or said organism from said phenotype to another phenotype, thereby identifying one or more nucleic acids associated with said phenotypic change;
(e) preparing two or more double-stranded oligoribonucleotides complementary to said nucleic acids identified in step (d) capable of modulating expression of said nucleic acids through RNA co-interference; and
(f) preparing an RNA co-interference composition comprising said nucleic acids prepared in accordance with step (e).

127. A method of designing a RNA co-interference composition of any one of claims 2 to 4 or 73 to 75, comprising:
(a) specifying a phenotype of interest associated with a disease, disorder, condition or trait affecting a cell, population of cells or organism;
(b) specifying a biochemical network of such cell, such population of cells or such organism postulated to be correlated to such specified phenotype;
(c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations;
(d) inferring additional biochemical pathways and nucleic acid targets in said cellular biochemical network by importing data into said mathematical equations;
(e) optimizing said simulated biochemical network by determining and constraining the values of said quantitative parameters and said imported data of said interrelationships;
(f) solving said mathematical equations to identify interrelationships likely to cause the transition of said cell, said population of cells or said organism from said phenotype to a second phenotype, thereby identifying one or more nucleic acids associated with said transition to said second phenotype;
(g) preparing two or more double-stranded oligoribonucleotides complementary to said nucleic acids identified in step (f) capable of modulating expression of said nucleic acids through RNA co-interference; and
(h) preparing an RNA co-interference composition comprising said nucleic acids prepared in accordance with step (g).

128. A method of designing a composition to modulate the expression of one or more target nucleic acids through RNA interference, comprising:
(a) specifying a phenotype of interest associated with a disease, disorder, condition or trait affecting a cell, population of cells or organism;
(b) specifying a biochemical network of such cell, such population of cells or such organism postulated to be correlated to such specified phenotype;
(c) simulating said biochemical network by (i) specifying the biochemical pathways of said biochemical network; (ii) identifying nucleic acid targets associated with said biochemical pathways; and (iii) representing said interrelationships between said biochemical pathways and said nucleic acid targets in one or more mathematical equations, wherein quantitative parameters of said interrelationships are set forth in said mathematical equations;
(d) inferring additional biochemical pathways and nucleic acid targets in said cellular biochemical network by importing data into said mathematical equations;
(e) optimizing said simulated biochemical network by determining and constraining the values of said quantitative parameters and said imported data of said interrelationships;
(f) solving said mathematical equations to identify interrelationships likely to cause the transition of said cell, said population of cells or said organism from said phenotype to a second phenotype, thereby identifying one or more nucleic acids associated with said transition to said second phenotype;
(g) preparing two or more double-stranded oligoribonucleotides complementary to said nucleic acids identified in step (f) capable of modulating expression of said nucleic acids through RNA co-interference; and
(h) preparing a composition comprising the double-stranded oligoribonucleotides prepared in accordance with step (g).

129. The method of claims 127, wherein qualitative parameters of said interrelationships are set forth in said mathematical equations in addition to quantitative parameters.

130. The method of claims 127, wherein in silico data is imported in step (d).

131. The method of claims 127, wherein in vitro data is imported in step (d).

132. The method of claims 127, wherein in vivo data is imported in step (d).

133. The method of claim 125, wherein step (e) identifies a synergistic interrelationship likely to cause transition of said cell, said population of cells or said organism from said phenotype to a second phenotype.

134. The method of claim 126, wherein step (d) identifies a synergistic interrelationship likely to cause transition of said cell, said population of cells or said organism from said phenotype to a second phenotype.

135. The method of claim 127, wherein step (f) identifies a synergistic interrelationship likely to cause transition of said cell, said population of cells or said organism from said phenotype to a second phenotype.

136. The method of claim 128, wherein step (f) identifies a synergistic interrelationship likely to cause transition of said cell, said population of cells or said organism from said phenotype to a second phenotype.

137. The method of claim 135, wherein the synergistic interrelationship involves a change in cellular behavior.

138. The method of claim 135, wherein the synergistic interrelationship involves is measured by a change in quantitative assay measurements of a one or more proteins or nucleic acids, or a cellular network.

139. The method of claim 127, wherein the phenotypic change occurs in a cell.

140. The method of claim 127, wherein the phenotypic change occurs in an organ.

141. The method of claim 127, wherein the phenotypic change occurs in an organism.

142. The method of claim 127, wherein the phenotypic change occurs in a cellular system.

143. The method of claim 127, wherein the phenotypic change results in modulation of expression of one or more proteins.

144. The method of claim 127, wherein the phenotypic change results in a change in protein activity.

145. The method of claim 127, wherein the synergistic change results in a change in protein activity.

146. The methods of any one of claims 2 to 4 or 73 to 75, wherein one or more mathematical equations are selected from the group consisting of: Institute for Systems Biology Measurement Approach, Genstruct Causal Modeling, Collins Mathematical Modeling, Entelos Mathematical Modeling, MNI#1, MNI#2, EQ1, EQ2, EQ3, EQ4, EQ5, EQ6, EQ7, EQ8, EQ9 and EQ10.


SEQUENCE LISTING (16 sequences)

SEQ ID NO: 1 - RNA, 19 nt, Homo sapiens
ggaucuuauuucuucggag

SEQ ID NO: 2 - RNA, 19 nt, Homo sapiens
cuccgaagaaauaagaucc

SEQ ID NO: 3 - DNA, 21 bp, Homo sapiens
ggcacgaguaacaagcucatt

SEQ ID NO: 4 - DNA, 21 bp, Homo sapiens
ugagcuuguuacucgugcctt

SEQ ID NO: 5 - RNA, 21 nt, Homo sapiens
aaaauggcuuugaaucuuugg

SEQ ID NO: 6 - DNA, 21 bp, Homo sapiens
aaagauucaaagccauuuutt

SEQ ID NO: 7 - DNA, 21 bp, Homo sapiens
gcaaaggaaaccaacaauatt

SEQ ID NO: 8 - DNA, 21 bp, Homo sapiens
uauuguugguuuccuuugctt

SEQ ID NO: 9 - DNA, 21 bp, Homo sapiens
ggucagagucuggaucacctt

SEQ ID NO: 10 - DNA, 21 bp, Homo sapiens
ggugauccagacucugacctt

SEQ ID NO: 11 - RNA, 25 nt, Homo sapiens
acucagaagagauaguaaugcucag

SEQ ID NO: 12 - RNA, 27 nt, Homo sapiens
cugagcauuacuaucucuucugaguuu

SEQ ID NO: 13 - DNA, 25 bp, Homo sapiens
gccaauuagacuugaaauacguutg

SEQ ID NO: 14 - RNA, 27 nt, Homo sapiens
caaacguauuucaagucuaauuggcuc

SEQ ID NO: 15 - RNA, 25 nt, Homo sapiens
gcacuaacauucuagaguauuccag

SEQ ID NO: 16 - RNA, 27 nt, Homo sapiens
cuggaauacucuagaauguuagugcug